SEQUENCE LISTING (15 sequences)

SEQ ID NO: 1 - protein, 10 aa, synthetic construct
  REGION 1..10: note = Synthetic Construct
RASSSVSYMH

SEQ ID NO: 2 - protein, 7 aa, synthetic construct
  REGION 1..7: note = Synthetic Construct
APSNLAS

SEQ ID NO: 3 - protein, 9 aa, synthetic construct
  REGION 1..9: note = Synthetic Construct
QQWSFNPPT

SEQ ID NO: 4 - protein, 10 aa, synthetic construct
  REGION 1..10: note = Synthetic Construct
GYTFTSYNMH

SEQ ID NO: 5 - protein, 17 aa, synthetic construct
  REGION 1..17: note = Synthetic Construct
AIYPGNGDTSYNQKFKG

SEQ ID NO: 6 - protein, 13 aa, synthetic construct
  REGION 1..13: note = Synthetic Construct
VVYYSNSYWYFDV

SEQ ID NO: 7 - protein, 107 aa, synthetic construct
  REGION 1..107: note = Synthetic Construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 8 - protein, 122 aa, synthetic construct [sequence omitted]

SEQ ID NO: 9 - protein, 213 aa, synthetic construct
  REGION 1..213: note = Synthetic Construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, synthetic construct
  REGION 1..452: note = Synthetic Construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 451 aa, synthetic construct
  REGION 1..451: note = Synthetic Construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 107 aa, synthetic construct
  REGION 1..107: note = Synthetic Construct
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKR

SEQ ID NO: 13 - protein, 108 aa, synthetic construct
  REGION 1..108: note = Synthetic Construct
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 14 - protein, 122 aa, synthetic construct
  REGION 1..122: note = Synthetic Construct
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSS

SEQ ID NO: 15 - protein, 119 aa, synthetic construct
  REGION 1..119: note = Synthetic Construct
EVQLVESGGGLVQPGGSLRLSCAASGFTFSSYAMSWVRQAPGKGLEWVAVISGDGGSTYYADSVKGRFTISRDNSKNTLTLQMNSLRAEDTAVYYCARGRVGYSLYDYWGQGTLVTVSS

(19) United States
(12) Patent Application Publication
(10) Pub. No.: US 2023/0322942 A1
(43) Pub. Date: Oct. 12, 2023

(54) METHODS FOR TREATING MULTIPLE SCLEROSIS WITH OCRELIZUMAB

(71) Applicant: Hoffmann-La Roche Inc., Little Falls, NJ (US)

(72) Inventor: Marianna MANFRINI, Basel (CH)

(73) Assignee: Hoffmann-La Roche Inc., Little Falls, NJ (US)

(21) Appl. No.: 18/339,960

(22) Filed: Jun. 22, 2023

Related U.S. Application Data
(63) Continuation of application No. 17/401,093, filed on Aug. 12, 2021.
(60) Provisional application No. 63/072,673, filed on Aug. 31, 2020.
(60) Provisional application No. 63/066,077, filed on Aug. 14, 2020.

(51) Int. Cl. C07K 16/28; A61P 25/28; A61K 39/395; A61K 45/06

(52) U.S. Cl. CPC C07K 16/2887; A61P 25/28; A61K 39/3955; A61K 45/06; A61K 2039/505

(57) ABSTRACT

The present invention concerns methods for treating multiple sclerosis (MS) in a patient, and an article of manufacture with instructions for such use.

### CROSS REFERENCE TO RELATED APPLICATIONS

This application is a continuation of U.S. patent application Ser. No. 17/401,093, filed Aug. 12, 2021, which claims the priority benefit of U.S. Provisional Application Ser. No. 63/066,077, filed Aug. 14, 2020 and U.S. Provisional Application Ser. No. 63/072,673, filed Aug. 31, 2020, the contents of which are incorporated herein by reference in their entirety.

### SUBMISSION OF SEQUENCE LISTING ON ASCII TEXT FILE

The contents of the electronic sequence listing (146392051101seqlist.xml; Size: 21,422 bytes; and Date of Creation: Jun. 12, 2023) are herein incorporated by reference in their entirety.

### FIELD OF THE INVENTION

The present invention concerns methods for treating multiple sclerosis (MS) in a patient, and an article of manufacture with instructions for such use.

### BACKGROUND OF THE INVENTION

Multiple sclerosis (MS) is a chronic, inflammatory, demyelinating, and degenerative disease of the central nervous system (CNS) that affects approximately 900,000 people in the United States (Wallin et al. (2019) Neurology. 92:e1029-40) and 2.3 million worldwide (GBD 2016 Multiple Sclerosis Collaborators. (2019) Lancet. 18:269-85). It is primarily a disease of young adults, with 70%-80% of patients having an age of onset (i.e., initial clinical presentation to a physician) between 20 and 40 years (Anderson et al. (1992) Ann Neurol 31:333-336; Noonan et al. (2002) Neurology. 58:136-138.) and has a gender bias influenced by the phenotype, with approximately up to 64%-70% of diagnosed patients being women.

MS is classified into three clinical phenotypes: relapsing remitting (RRMS), secondary progressive (SPMS), and primary progressive (PPMS) (Lublin et al. (2014) Neurology. 83:278-86). These three phenotypes are further subdivided into active and non-active forms based on the presence or absence of disease activity, defined by the presence of clinical relapses and/or so-called active lesions on a magnetic resonance imaging (MRI) scan. Active MRI lesions are gadolinium-enhancing lesions on T1-weighted scan (T1Gd⁺) or new T2-weighted lesions/enlarging T2-weighted lesions. Relapsing MS (RMS) forms encompass RRMS and active SPMS, and progressive MS (PMS) forms constitute non-active SPMS and PPMS.

Evidence available to date suggests that despite the potential heterogeneity of the clinical expression of the disease, PPMS, SPMS, and RRMS belong to the same disease spectrum, and that pathological mechanisms responsible for relapses/disease activity and progression biology are largely identical across the MS spectrum (Lassmann (2018) Cold Spring Harb Perspect Med. 8(3):a028936). Although the mechanisms associated with disease progression are assumed to be present from the onset of the disease (Cree et al. (2019) Curr Opin Neurol. 32: 365-77), clinical disability progression manifests often later in the course of a patient's disease most likely due to the degree of brain reserve of the patient. The symptomatic worsening associated with MS disability progression results in a slow, insidious loss of a patient's motor and sensory function, as well as cognitive decline and autonomic dysfunctions.

Disability progression across the spectrum of MS might occur as a result of two concurrent inflammatory mechanisms: acute inflammation and chronic compartmentalized inflammation.

Acute inflammation can be observed on an MRI scan (as T1Gd⁺ lesions or new T2 lesions/enlarging T2 lesions) and clinically manifests as relapses, where it can also lead to step-wise increase of disability due to incomplete relapse recovery. Pathophysiologically, relapsing forms of MS (i.e., RMS) are associated with focal T-cell and B-cell invasion, with blood brain barrier leakage that give rise to classic active demyelinating plaques in the white matter. However, RMS also harbors signs of progression biology/chronic compartmentalized inflammation.

By contrast to these acute inflammatory processes, chronic compartmentalized inflammation is responsible for an increase in disability that occurs independently from relapses or radiological disease activity and is characterized by demyelination and axonal loss (progression biology). Progressive forms of MS (i.e., PMS) are associated with a chronic and slow accumulation of T cells and B cells in the connective tissue spaces of the brain, without leakage of the blood brain barrier. There is a typical formation of subpial-demyelinated lesions in the cerebral and cerebellar cortex, with slow expansion of pre-existing lesions in the white matter and diffuse chronic inflammation in the normal appearing white or gray matter.

Even though there are many drugs currently available that target the acute inflammatory mechanisms associated with relapses and relapse associated worsening, to date, only ocrelizumab is indicated for PPMS (note: ocrelizumab is only approved for active PPMS [aPPMS] in some countries). As a result, the salient feature of disability progression in all forms of MS remains to be further addressed, and treatments that can stop or delay MS disease progression represent a serious unmet medical need.

All references cited herein are incorporated by reference in their entirety.

### BRIEF SUMMARY OF THE INVENTION

In some embodiments, provided is method of treating multiple sclerosis in a patient comprising administering an effective amount of an anti-CD20 antibody to the patient to provide an initial anti-CD20 antibody dose of about 1.2 grams followed by a second anti-CD20 antibody dose of about 1.2 grams, the second dose not being provided until from about 24 weeks from the initial dose, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, and wherein the patient weighs less than about 75 kg at the time of the first anti-CD20 antibody dose.

In some embodiments, provided is a method of treating multiple sclerosis in a patient comprising administering an effective amount of an anti-CD20 antibody to the patient to provide an initial anti-CD20 antibody dose of about 1.2 grams followed by a second anti-CD20 antibody dose of about 1.2 grams, the second dose not being provided until from about 6 months from the initial dose, wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, and wherein the patient weighs less than about 75 kg at the time of the first anti-CD20 antibody dose.

In some embodiments, the initial anti-CD20 antibody dose comprises a first intravenous (IV) infusion and a second IV infusion of the anti-CD20 antibody, wherein the first IV infusion and second IV infusion of the anti-CD20 antibody are each about 0.6 grams. In some embodiments, the initial anti-CD20 antibody dose comprises a single IV infusion of the anti-CD20 antibody, wherein the single IV infusion of the anti-CD20 antibody is about 1.2 grams. In some embodiments, the second anti-CD20 dose comprises a single IV infusion of the anti-CD20 antibody, wherein the single IV fusion of the anti-CD20 antibody is about 1.2 grams.

In some embodiments, provided is a method of treating multiple sclerosis in a patient comprising administering an effective amount of an anti-CD20 antibody to the patient to provide an initial anti-CD20 antibody dose of about 1.8 grams followed by a second anti-CD20 antibody dose of about 1.8 grams, the second dose not being provided until from about 24 weeks from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs about 75 kg or more at the time of the first anti-CD20 antibody dose.

In some embodiments, provided is a method of treating multiple sclerosis in a patient comprising administering an effective amount of an anti-CD20 antibody to the patient to provide an initial anti-CD20 antibody dose of about 1.8 grams followed by a second anti-CD20 antibody dose of about 1.8 grams, the second dose not being provided until from about 6 months from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs about 75 kg or more at the time of the first anti-CD20 antibody dose.

In some embodiments, the initial anti-CD20 antibody dose comprises a first intravenous (IV) infusion and a second IV infusion of the anti-CD20 antibody, wherein the first IV infusion and second IV infusion of the anti-CD20 antibody are each about 0.9 grams. In some embodiments, the initial anti-CD20 antibody dose comprises a single IV infusion of the anti-CD20 antibody, wherein the single IV infusion of the anti-CD20 antibody is about 1.8 grams. In some embodiments, the second anti-CD20 antibody dose comprises a single IV infusion of the anti-CD20 antibody, wherein the single IV infusion of the anti-CD20 antibody is about 1.8 grams.

In some embodiments, the second IV infusion is administered from about 3 to 17 days from the time the first IV infusion is administered. In some embodiments, the second IV infusion is administered from about 6 to 16 days from the time the first IV infusion is administered. In some embodiments, the second IV infusion is administered from about 13 to 16 days from the time the first IV infusion is administered. In some embodiments, the second IV infusion is administered 14 days from the time the first IV infusion is administered. In some embodiments, the second IV infusion is administered two weeks from the time the first IV infusion is administered.

In some embodiments, the method further comprises providing a third anti-CD20 antibody dose. In some embodiments, the third anti-CD20 antibody dose is provided about 24 weeks from the second dose. In some embodiments, the third anti-CD20 antibody dose is provided about 6 months from the second dose. In some embodiments, the method further comprises providing a fourth anti-CD20 antibody dose. In some embodiments, the fourth anti-CD20 antibody dose is provided about 24 weeks from the third dose. In some embodiments, the fourth anti-CD20 antibody dose is provided about 6 months from the third dose. In some embodiments, the method further comprises providing a fifth anti-CD20 antibody dose. In some embodiments, the fifth anti-CD20 antibody dose is provided about 24 weeks from the fourth dose. In some embodiments, the fifth anti-CD20 antibody dose is provided about 6 months from the fourth dose. In some embodiments, subsequent anti-CD20 antibody doses following the fifth anti-CD20 antibody dose are administered at intervals of about 24 weeks. In some embodiments, subsequent anti-CD20 antibody doses following the fifth anti-CD20 antibody dose are administered at intervals of about 6 months.

In some embodiments, the anti-CD20 antibody comprises a light chain comprising the amino acid sequence of SEQ ID NO: 9 and a heavy chain comprising the amino acid sequence of SEQ ID NO: 11. In some embodiments, the anti-CD20 antibody is ocrelizumab.

In some embodiments, the multiple sclerosis is relapsing multiple sclerosis (RMS). In some embodiments, the patient has RMS, and treatment results in reduced risk of 12-week composite confirmed disability progression (cCDP 12). In some embodiments, the patient has RMS, and treatment results (or further results) in one or more of: (a) increase in time to onset of 24-week cCDP; (b) increase in time to onset of 12-week confirmed disability progression (CDP); (c) increase in time to onset of 24-week CDP; (d) increase in time to >20% increase in 12-week confirmed timed 25 foot walk test (T25FWT); (e) increase in time to >20% increase in 24-week confirmed T25FWT; (f) decrease in the percent change in total brain volume after 24, 48, 72, 96, and 120 weeks of treatment; and (g) increase in time to 12-week confirmed 4-point worsening in Symbol Digital Modality Test (SDMT). In some embodiments, the patient has RMS, and treatment results (or further results) in one or more of: (A) reduction or no change in Expanded Disability Status Scare (EDSS) score; (B) increase in time to >20% increase in 12-week confirmed 9-hole peg test (9-HPT); (C) increase in time to >20% increase in 24-week confirmed 9-HPT; (D) increase in time to onset of cCDP 12 and progression in cCDP individual components independent of relapses; (E) reduction in new T1-hypointense lesions; (F) reduction in volume of T1-hypointense lesions; (G) reduction in spinal cord volume loss; (H) reduction in annualized relapse rate (ARR); (I) increase in time to onset of 12-week confirmed relapse-associated worsening (RAW) and individual components; (J) reduction in number of new or enlarging T2 lesions over treatment period; and (K) reduction in number of T1 Gd⁺ staining lesions over treatment period.

In some embodiments, the multiple sclerosis is primary progressive multiple sclerosis (PPMS). In some embodiments, the patient has PPMS, and treatment results in reduced risk of 12-week composite confirmed disability progression (cCDP 12). In some embodiments, the patient has PPMS, and treatment results (or further results) in one or more of: (a) increase in time to onset of 24-week cCDP; (b) increase in time to onset of 12-week confirmed disability progression (CDP); (c) increase in time to onset of 24-week CDP; (d) increase in time to >20% increase in 12-week confirmed timed 25 foot walk test (T25FWT); (e) increase in time to >20% increase in 24-week confirmed T25FWT; (f) increase in time to >20% increase in 12-week confirmed 9-hole peg test (9-HPT); (g) increase in time to >20% increase in 24-week confirmed 9-HPT; (h) decrease in loss of total brain volume during over treatment period following second ant-CD20 antibody dose; and (i) increase in time to 12-week confirmed 4-point worsening in Symbol Digital Modality Test (SDMT). In some embodiments, the patient has PPMS, and treatment results (or further results) in one or more of: (A) a reduction or no change in Expanded Disability Status Scare (EDSS) score; (B) reduction in new T1-hypointense lesions; (C) reduction in volume of T1-hypointense lesions; (D) reduction in spinal cord volume loss; (E) reduction in number of new or enlarging T2 lesions over treatment period; and (F) reduction in number of T1 Gd+ staining lesions over treatment period.

In some embodiments, a second medicament is administered to the patient with the initial anti-CD20 antibody dose or later anti-CD20 antibody doses, wherein the anti-CD20 antibody is the first medicament. In some embodiments, the second medicament is selected from the group consisting of an interferon, glatiramer acetate, a cytotoxic agent, a chemotherapeutic agent, mitoxantrone, methotrexate, cyclophosphamide, chlorambucil, azathioprine, gamma globulin, Campath, anti-CD4, cladribine, corticosteroid, mycophenolate mofetil (MMF), cyclosporine, a cholesterol-lowering drug of the statin class, estradiol, testosterone; a hormone replacement drug, a TNF inhibitor, a disease-modifying anti-rheumatic drug (DMARD), a non-steroidal anti-inflammatory drug (NSAID), levothyroxine, cyclosporin A, a somatastatin analogue, a cytokine or cytokine receptor antagonist, an anti-metabolite, an immunosuppressive agent, an integrin antagonist or antibody, an LF A −1 antibody, efalizumab, an alpha 4 integrin antibody, natalizumab, and another B-cell surface marker antibody. In some embodiments, the patient has never been previously treated with an anti-CD20 antibody. In some embodiments, the patient has received prior treatment with an anti-CD20 antibody In some embodiments, the anti-CD20 antibody is the only medicament administered to the patient to treat multiple sclerosis.

In some embodiments, provided is an article of manufacture comprising: (a) a container comprising an anti-CD20 antibody, which anti-CD20 antibody comprises a VH domain comprising the amino acid set forth in SEQ ID NO: 8, a VL domain comprising the amino acid sequence set forth in SEQ ID NO: 7, and a human IgG1 constant region; and (b) a package insert with instructions for treating multiple sclerosis in a patient according to any one of the preceding claims.

It is to be understood that one, some, or all of the properties of the various embodiments described herein may be combined to form other embodiments of the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a sequence alignment comparing the amino acid sequences of the light chain variable domain (V_{L}) of each of murine 2H7 (SEQ ID NO:12), humanized 2H7.v16 variant (SEQ ID NO:7), and the human kappa light chain subgroup I (SEQ ID NO:13). The CDRs of V_{L} of 2H7 and hu2H7.v16 are as follows: CDR1 (SEQ ID NO:1), CDR2 (SEQ ID NO:2), and CDR3 (SEQ ID NO:3).

FIG. 1B is a sequence alignment comparing the amino acid sequences of the heavy chain variable domain (V_{H}) of each of murine 2H7 (SEQ ID NO:14), humanized 2H7.v16 variant (SEQ ID NO:8), and the human consensus sequence of the heavy chain subgroup III (SEQ ID NO:15). The CDRs of V_{H} of 2H7 and hu2H7.v16 are as follows: CDR1 (SEQ ID NO:4), CDR2 (SEQ ID NO:5), and CDR3 (SEQ ID NO:6).

In FIGS. 1A and 1B, the CDR1, CDR2 and CDR3 in each chain are enclosed within brackets, flanked by the framework regions, FR1-FR4, as indicated. 2H7 refers to the murine 2H7 antibody. The asterisks in between two rows of sequences indicate the positions that are different between the two sequences. Residue numbering is according to Kabat et al. Sequences of Immunological Interest, 5th Ed. Public Health Service, National Institutes of Health, Bethesda, Md. (1991), with insertions shown as a, b, c, d, and e.

FIG. 2 provides a goodness of fit for the final Model (RMS). (DV: Observed concentrations; PRED: population predictions of the model; IPRED: individual predictions of the model; CWRES: conditional weighted residuals; CIWRES: individual weighted residuals; TIME: time after the first dose; TAD: time after the most recent dose. The gray solid y=x or y=0 lines are included for reference. The bold red lines are the lowers (local regression smoother) trend lines.

FIG. 3 provides a visual predictive Check, Semi-Log Scale, RMS. The lines show median (red), and the 5th and 95th percentiles (blue) of the observed concentrations (circles). The shaded regions show the 90% confidence intervals on these quantities obtained by simulations. The simulated values were computed from 1000 trials with dosing, sampling, and the covariate values of the analysis dataset. Time point: for studies 21092 and 21093: 1=Nominal Day (NDAY) 169, 2=NDAY 337, 3=NDAY 505 pre-dose, 4=NDAY 505 post-dose, 5=NDAY 589, 6=NDAY 673; for study 21493 Part 1: 1=NDAY 1 post-dose, 2=NDAY 15 pre-dose, 3=NDAY 15 post-dose, 4=NDAY 29, 5=NDAY 57, 6=NDAY 85, 7=NDAY 113, 8=NDAY 141, 9=NDAY 169 pre-dose; for study 21493 Part 2: 1=NDAY 15, 2=NDAY 169, 3=NDAY 337, 4=NDAY 505, 6=NDAY 673, 6=NDAY 841.

FIG. 4 provides normalized prediction distribution errors (NPDE) for the final model, RMS. Circles correspond to NPDE of observations in the distribution of 1000 simulated values. Lines at y=0 correspond to median, and dashed lines show the 10th and 90th percentiles. Red lines show the lowess trend lines. SEX: 1=Males, 2=Females.

FIG. 5 provides goodness of Fit, PPMS (DV: Observed concentrations; PRED: population predictions of the model; IPRED: individual predictions of the model; CWRES: conditional weighted residuals; CIWRES: individual weighted residuals; TIME: time after the first dose; TAD: time after the most recent dose. The gray solid y=x or y=0 lines are included for reference. The bold red lines are the lowess (local regression smoother) trend lines).

FIG. 6 provides a Visual Predictive Check, Semi-Log Scale, PPMS. The lines show median (red), and the 5th and 95th percentiles (blue) of the observed concentrations (circles). The shaded regions show the 90% confidence intervals on these quantities obtained by simulations. The simulated values were computed from 1000 trials with dosing, sampling, and the covariate values of the analysis dataset.

FIG. 7 provides normalized prediction distribution errors (NPDE), PPMS. Circles correspond to NPDE of observations in the distribution of 1000 simulated values. Lines at y=0 correspond to median, and dashed lines show the 10th and 90th percentiles. Red lines show the lowess trend lines. SEX: 1=Males, 2=Females.

FIG. 8A shows the proportion of patients with RMS (Phase III trials WA21092 and WA21093) with a B-cell count of ≤5 cells/μL in blood by ocrelizumab Cₘₑₐₙ exposure quartiles over time. In patients with RMS, Cₘₑₐₙ quartile ranges (μg/mL) were: Q1: Min-15.38; Q2: 15.38-18.72; Q3: 18.72-22.17; Q4: 22.17—Max, and median (range) body weights (kg) were: Q1: 89 (49-170); Q2: 79 (49-123); Q3: 67 (46-108); Q4: 60 (38-97). Cₘₑₐₙ, mean concentration over time; OCR, ocrelizumab; PPMS, primary progressive multiple sclerosis; Q, quartile; RMS, relapsing multiple sclerosis.

FIG. 8B shows the proportion of patients with PPMS (Phase III trial WA25046) with a B-cell count of ≤5 cells/μL in blood by ocrelizumab Cₘₑₐₙ exposure quartiles over time. In patients with PPMS, Cₘₑₐₙ quartile ranges (μg/mL) were: Q1: Min-15.83; Q2: 15.83-18.92; Q3: 18.92-23.15; Q4: 23.15—Max, and median (range) body weights (kg) were: Q1: 84 (46-136); Q2: 74 (46-125); Q3: 68 (46-115); Q4: 56 (40-93). Cₘₑₐₙ, mean concentration over time; OCR, ocrelizumab; PPMS, primary progressive multiple sclerosis; Q, quartile; RMS, relapsing multiple sclerosis.

FIG. 9 provides a schematic for a Phase IIIb randomized, double blind, controlled, parallel group study to evaluate the efficacy and safety of a higher dose of ocrelizumab in patients with relapsing multiple sclerosis (RMS).

FIG. 10 provides a schematic for a Phase IIIb randomized, double blind, controlled, parallel group study to evaluate the efficacy and safety of a higher dose of ocrelizumab in patients with primary progressive multiple sclerosis (PPMS).

FIG. 11A shows an exposure-response analysis and forest plot of 24 week confirmed disability progression (24 W-CDP) in patients with RMS.

FIG. 11B shows an exposure-response analysis and forest plot of 24 week confirmed disability progression (24 W-CDP) in patients with PPMS.

FIG. 12A shows a modelled relationship between OCR exposure and 12 week composite confirmed disability progression (12 w cCDP) in patients with RMS.

FIG. 12B shows a modelled relationship between OCR exposure and 12 week composite confirmed disability progression (12 w cCDP) in patients with PPMS.

FIG. 13A shows modelled exposure distributions for the approved OCR 600 mg and higher-dose regimens in patients with RMS.

FIG. 13B shows modelled exposure distributions for the approved OCR 600 mg and higher-dose regimens in patients with PPMS.

### DETAILED DESCRIPTION OF THE INVENTION

### Definitions

A “B-cell” is a lymphocyte that matures within the bone marrow, and includes a naive B cell, memory B cell, or effector B cell (plasma cells). The B-cell herein may be a normal or non-malignant B cell.

A “B-cell surface marker” or “B-cell surface antigen” herein is an antigen expressed on the surface of a B cell that can be targeted with an antibody that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers (for descriptions, see The Leukocyte Antigen Facts Book, 2^{nd} Edition. 1997, ed. Barclay et al. Academic Press, Harcourt Brace & Co., New York). Other B-cell surface markers include RP105, FcRH2, B-cell CR2, CCR6, P2X5, HLA-DOB, CXCR5, FCER2, BR3, Btig, NAG14, SLGC16270, FcRH1, IRTA2, ATWD578, FcRH3, IRTA1, FcRH6, BCMA, and 239287. The B-cell surface marker of particular interest herein is preferentially expressed on B cells compared to other non-B-cell tissues of a mammal and may be expressed on both precursor B cells and mature B cells. The preferred B-cell surface marker herein is CD20.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al. Proc. Natl. Acad. Sci. (USA) 82:1766 (1985), for example.

An “antibody antagonist” herein is an antibody that, upon binding to a B cell surface marker on B cells, destroys or depletes B cells in a mammal and/or interferes with one or more B-cell functions, e.g. by reducing or preventing a humoral response elicited by the B cell. The antibody antagonist preferably is able to deplete B cells (i.e. reduce circulating B-cell levels) in a mammal treated therewith. Such depletion may be achieved via various mechanisms such antibody-dependent cell-mediated cytotoxicity (ADCC) and/or complement dependent cytotoxicity (CDC), inhibition of B-cell proliferation and/or induction of B-cell death (e.g. via apoptosis). The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

“Antibodies” or “native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from mammalian species can be assigned to one of two clearly distinct types, called kappa (κ) and lambda (λ), based on the amino acid sequences of their constant domains.

The “heavy chains” of antibodies from mammalian species can also be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

The term “ocrelizumab” (CAS Registration No. 637334-45-3) herein refers to the genetically engineered humanized monoclonal antibody directed against the CD20 antigen and comprising (a) a light chain comprising the amino acid sequence of SEQ ID NO: 9 and (b) a heavy chain comprising the amino acid sequence of SEQ ID NO: 11, including fragments thereof that retain the ability to bind CD20. Ocrelizumab is available from Genentech.

A “subject” or “patient” herein is a human subject or patient. Generally, the subject or patient is eligible for treatment for multiple sclerosis. For the purposes herein, such eligible subject or patient is one who is experiencing, has experienced, or is likely to experience, one or more signs, symptoms or other indicators of multiple sclerosis; has been diagnosed with multiple sclerosis, whether, for example, newly diagnosed (with “new onset” MS), previously diagnosed with a new relapse or exacerbation, previously diagnosed and in remission, etc.; and/or is at risk for developing multiple sclerosis. One suffering from or at risk for suffering from multiple sclerosis may optionally be identified as one who has been screened for elevated levels of CD20-positive B cells in serum, cerebrospinal fluid (CSF) and/or MS lesion(s) and/or is screened for using an assay to detect autoantibodies, assessed qualitatively, and preferably quantitatively. Exemplary such autoantibodies associated with multiple sclerosis include anti-myelin basic protein (MBP), anti-myelin oligodendrocytic glycoprotein (MOG), anti-ganglioside and/or anti-neurofilament antibodies. Such autoantibodies may be detected in the subject's serum, cerebrospinal fluid (CSF) and/or MS lesion. By “elevated” autoantibody or B cell level(s) herein is meant level(s) of such autoantibodies or B cells which significantly exceed the level(s) in an individual without MS.

As used herein, “treatment” or “treating” is an approach for obtaining beneficial or desired results including clinical results. For purposes of this invention, beneficial or desired clinical results include, but are not limited to, one or more of the following: decreasing one or more symptoms resulting from the disease, diminishing the extent of the disease, stabilizing the disease (e.g., preventing or delaying the worsening of the disease), delay or slowing the progression of the disease, ameliorating the disease state, decreasing the dose of one or more other medications required to treat the disease, and/or increasing the quality of life.

As used herein, “delaying” or “slowing” the progression of multiple sclerosis means to prevent, defer, hinder, slow, retard, stabilize, and/or postpone development of the disease. This delay can be of varying lengths of time, depending on the history of the disease and/or individual being treated.

As used herein, “at the time of starting treatment” refers to the time period at or prior to the first dose of a multiple sclerosis drug, such as an anti-CD20 antibody. In some embodiments, “at the time of starting treatment” is about any of one year, nine months, six months, three months, second months, or one month prior to a multiple sclerosis drug, such as an anti-CD20 antibody. In some embodiments, “at the time of starting treatment” is immediately prior to coincidental with the first dose of a multiple sclerosis drug, such as an anti-CD20 antibody.

As used herein, “based upon” includes (1) assessing, determining, or measuring the patient characteristics as described herein (and preferably selecting a patient suitable for receiving treatment; and (2) administering the treatment(s) as described herein.

A “symptom” of MS is any morbid phenomenon or departure from the normal in structure, function, or sensation, experienced by the subject and indicative of MS.

“Multiple sclerosis” refers to the chronic inflammatory, often disabling disease of the central nervous system characterized by demyelination and neurodegeneration. There are three internationally recognized forms of MS, namely, primary progressive multiple sclerosis (PPMS), relapsing-remitting multiple sclerosis (RRMS), and secondary progressive multiple sclerosis (SPMS).

“Progressive multiple sclerosis” as used herein refers to primary progressive multiple sclerosis (PPMS), and secondary progressive multiple sclerosis (SPMS). In some embodiments, progressive multiple sclerosis is characterized by documented, irreversible loss of neurological function persisting for ≥6 months that cannot be attributed to clinical relapse.

“Primary progressive multiple sclerosis” or “PPMS” is characterized by a gradual progression of the disease from its onset with rare superimposed relapses and remissions. There may be periods of a leveling off of disease activity and there may be good and bad days or weeks. PPMS differs from RRMS and SPMS in that onset is typically in the late thirties or early forties, men are as likely women to develop it, and initial disease activity is often in the spinal cord and not in the brain. PPMS disease activity can also be observed (or found) in the brain. PPMS is the sub-type of MS that is least likely to show inflammatory (gadolinium enhancing) lesions on MRI scans. The Primary Progressive form of the disease affects about 15% of all people with multiple sclerosis. PPMS may be defined according to the criteria in Thompson et al. (2018) Lancet 7(2):162-173. The subject with PPMS treated herein is usually one with probable or definitive diagnosis of PPMS.

“Relapsing-remitting multiple sclerosis” or “RRMS” is characterized by relapses (also known as exacerbations) during which time new symptoms can appear and old ones resurface or worsen. The relapses are followed by periods of remission, during which time the person fully or partially recovers from the deficits acquired during the relapse. Relapses can last for days, weeks or months and recovery can be slow and gradual or almost instantaneous. The vast majority (about 85%) of people presenting with MS are first diagnosed with RRMS. This is typically when they are in their twenties or thirties, though diagnoses much earlier or later are known. Twice as many women as men present with this sub-type of MS. During relapses, myelin, a protective insulating sheath around the nerve fibers (neurons) in the white matter regions of the central nervous system (CNS), may be damaged in an inflammatory response by the body's own immune system. This causes a wide variety of neurological symptoms that vary considerably depending on which areas of the CNS are damaged. Immediately after a relapse, the inflammatory response dies down and a special type of glial cell in the CNS (called an oligodendrocyte) sponsors remyelination—a process whereby the myelin sheath around the axon may be repaired. It is this remyelination that may be responsible for the remission. Approximately 50% of patients with RRMS convert to SPMS within 10 years of disease onset. After 30 years, this figure rises to 90%. At any one time, the relapsing-remitting form of the disease accounts around 55% of all people with MS.

In some embodiments, an initial or first “antibody dose” refers to contact with or exposure to the antibody herein in one or more infusions administered over a period of time of about 1-20 days. The infusions may be given at one time or at fixed or irregular time intervals over this period of exposure. Initial and later (e.g. second or third) antibody doses are separated in time from each other as described in detail herein.

As used herein, an “interval” between antibody doses refers to time period between an earlier antibody dose and a later antibody dose. An antibody dose of the present disclosure may include one or two infusions (e.g., intravenous (IV) infusions). In cases where the antibody dose contain one infusion, an interval between two antibody doses refers to the amount of time elapsed between the infusion of one antibody dose (e.g., Day 1) and the infusion of the next antibody dose. If one antibody dose includes two infusions and the next antibody dose includes one infusion, an interval between the two antibody doses refers to the amount of time elapsed between the first of the two infusions of the first antibody dose (e.g., Day 1) and the infusion of the next antibody dose. In cases where each of the two antibody doses comprise two infusions, an interval between to the antibody doses refers to the amount of time elapsed between the first of the two infusions of the first antibody dose (e.g., Day 1) and the first infusion of the two infusions of the second antibody dose. For example, if a method of the present disclosure includes a first antibody dose with two infusions and a second antibody dose with two infusions, and the second antibody dose is not provided until about 24 weeks or 6 months after the first antibody dose, then the interval between the first infusion of the first antibody dose and the first infusion of the second antibody dose is about 24 weeks or 6 months.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone), dexamethasone, glucocorticoid and betamethasone.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

A “label” is used herein to refer to information customarily included with commercial packages of pharmaceutical formulations including containers such as vials and package inserts, as well as other types of packaging.

Reference to “about” a value or parameter herein includes (and describes) variations that are directed to that value or parameter per se. For example, description referring to “about X” includes description of “X.”

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

It is to be understood that one, some, or all of the properties of the various embodiments described herein may be combined to form other embodiments of the present invention. These and other aspects of the invention will become apparent to one of skill in the art.

All references cited herein, including patent applications and publications, are incorporated by reference in their entirety.

### Methods of Treatment

In certain embodiments, provided is a method of treating multiple sclerosis in a patient, comprising administering an effective amount of an anti-CD20 antibody to the patient to provide an initial anti-CD20 antibody dose of about 1.2 grams followed by a second anti-CD20 antibody dose of about 1.2 grams, the second dose not being provided until from about 24 weeks from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs less than about 75 kg at the time of the first anti-CD20 antibody dose.

In certain embodiments, provided is a method of treating multiple sclerosis in a patient, comprising administering an effective amount of an anti-CD20 antibody to the patient to provide an initial anti-CD20 antibody dose of about 1.2 grams followed by a second anti-CD20 antibody dose of about 1.2 grams, the second dose not being provided until from about 24 weeks from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs about 75 kg or less at the time of the first anti-CD20 antibody dose.

In certain embodiments, provided is a method of treating multiple sclerosis in a patient, comprising administering an effective amount of an anti-CD20 antibody to the patient to provide an initial anti-CD20 antibody dose of about 1.2 grams followed by a second anti-CD20 antibody dose of about 1.2 grams, the second dose not being provided until from about 6 months from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs less than about 75 kg at the time of the first anti-CD20 antibody dose.

In certain embodiments, provided is a method of treating multiple sclerosis in a patient, comprising administering an effective amount of an anti-CD20 antibody to the patient to provide an initial anti-CD20 antibody dose of about 1.2 grams followed by a second anti-CD20 antibody dose of about 1.2 grams, the second dose not being provided until from about 6 months from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs about 75 kg or less at the time of the first anti-CD20 antibody dose.

In certain embodiments, provided is a method of treating multiple sclerosis in a patient, comprising administering an effective amount of an anti-CD20 antibody to the patient to provide an initial anti-CD20 antibody dose of about 1.2 grams followed by a second anti-CD20 antibody dose of about 1.2 grams, the second dose not being provided until from about 24 weeks from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs less than about 70 kg at the time of the first anti-CD20 antibody dose.

In certain embodiments, provided is a method of treating multiple sclerosis in a patient, comprising administering an effective amount of an anti-CD20 antibody to the patient to provide an initial anti-CD20 antibody dose of about 1.2 grams followed by a second anti-CD20 antibody dose of about 1.2 grams, the second dose not being provided until from about 24 weeks from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs about 70 kg or less at the time of the first anti-CD20 antibody dose.

In certain embodiments, provided is a method of treating multiple sclerosis in a patient, comprising administering an effective amount of an anti-CD20 antibody to the patient to provide an initial anti-CD20 antibody dose of about 1.2 grams followed by a second anti-CD20 antibody dose of about 1.2 grams, the second dose not being provided until from about 6 months from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs less than about 70 kg at the time of the first anti-CD20 antibody dose. nti-CD20 antibody dose.

In certain embodiments, provided is a method of treating multiple sclerosis in a patient, comprising administering an effective amount of an anti-CD20 antibody to the patient to provide an initial anti-CD20 antibody dose of about 1.2 grams followed by a second anti-CD20 antibody dose of about 1.2 grams, the second dose not being provided until from about 6 months from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs about 70 kg or less at the time of the first anti-CD20 antibody dose. nti-CD20 antibody dose.

In some embodiments, the initial anti-CD20 antibody dose comprises a first intravenous infusion (e.g., intravenous (IV) infusion) and a second infusion of anti-CD20 antibody, wherein the first infusion and second infusion of anti-CD20 antibody are each about 0.6 grams. In some embodiments, the second infusion is administered from about 3 to 17 days from the time the first infusion was administered. In some embodiments, the second infusion is administered from about 6 to 16 days from the time the first infusion was administered. In some embodiments, the second infusion is administered from about 13 to 16 days from the time the first infusion was administered. In some embodiments, the second IV infusion is administered 14 days from the time the first IV infusion was administered. In some embodiments, the second IV infusion is administered two weeks from the time the first IV infusion was administered. In some embodiments the terms “14 days” and “2 weeks” are used interchangeably. In some embodiments, the initial anti-CD20 antibody dose comprises a single infusion of anti-CD20 antibody, wherein the single infusion of anti-CD20 antibody is about 1.2 grams. In some embodiments, the second anti-CD20 antibody dose comprises a single infusion of anti-CD20 antibody, wherein the single infusion of anti-CD20 antibody is about 1.2 grams. In some embodiments, the second dose is not administered less than about 20 weeks after the first dose.

In some embodiments, provided is a method of treating multiple sclerosis in a patient comprising administering an effective amount of an anti-CD20 antibody to the patient to provide an initial anti-CD20 antibody dose of about 1.8 grams followed by a second anti-CD20 antibody dose of about 1.8 grams, the second dose not being provided until from about 24 weeks from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs about 75 kg or more at the time of the first anti-CD20 antibody dose.

In some embodiments, provided is a method of treating multiple sclerosis in a patient comprising administering an effective amount of an anti-CD20 antibody to the patient to provide an initial anti-CD20 antibody dose of about 1.8 grams followed by a second anti-CD20 antibody dose of about 1.8 grams, the second dose not being provided until from about 24 weeks from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs more than about 75 kg at the time of the first anti-CD20 antibody dose.

In some embodiments, provided is a method of treating multiple sclerosis in a patient comprising administering an effective amount of an anti-CD20 antibody to the patient to provide an initial anti-CD20 antibody dose of about 1.8 grams followed by a second anti-CD20 antibody dose of about 1.8 grams, the second dose not being provided until from about 6 months from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs about 75 kg or more at the time of the first anti-CD20 antibody dose.

In some embodiments, provided is a method of treating multiple sclerosis in a patient comprising administering an effective amount of an anti-CD20 antibody to the patient to provide an initial anti-CD20 antibody dose of about 1.8 grams followed by a second anti-CD20 antibody dose of about 1.8 grams, the second dose not being provided until from about 6 months from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs more than about 75 kg at the time of the first anti-CD20 antibody dose.

In some embodiments, provided is a method of treating multiple sclerosis in a patient comprising administering an effective amount of an anti-CD20 antibody to the patient to provide an initial anti-CD20 antibody dose of about 1.8 grams followed by a second anti-CD20 antibody dose of about 1.8 grams, the second dose not being provided until from about 24 weeks from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs about 70 kg or more at the time of the first anti-CD20 antibody dose.

In some embodiments, provided is a method of treating multiple sclerosis in a patient comprising administering an effective amount of an anti-CD20 antibody to the patient to provide an initial anti-CD20 antibody dose of about 1.8 grams followed by a second anti-CD20 antibody dose of about 1.8 grams, the second dose not being provided until from about 24 weeks from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs more than about 70 kg at the time of the first anti-CD20 antibody dose.

In some embodiments, provided is a method of treating multiple sclerosis in a patient comprising administering an effective amount of an anti-CD20 antibody to the patient to provide an initial anti-CD20 antibody dose of about 1.8 grams followed by a second anti-CD20 antibody dose of about 1.8 grams, the second dose not being provided until from about 6 months from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs about 70 kg or more at the time of the first anti-CD20 antibody dose.

In some embodiments, provided is a method of treating multiple sclerosis in a patient comprising administering an effective amount of an anti-CD20 antibody to the patient to provide an initial anti-CD20 antibody dose of about 1.8 grams followed by a second anti-CD20 antibody dose of about 1.8 grams, the second dose not being provided until from about 6 months from the initial dose, wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and wherein the patient weighs more than about 70 kg at the time of the first anti-CD20 antibody dose.

In some embodiments, the initial anti-CD20 antibody dose comprises a first intravenous (IV) infusion and a second IV infusion of anti-CD20 antibody, wherein the first IV infusion and second IV infusion of anti-CD20 antibody are each about 0.9 grams. In some embodiments, the second infusion is administered from about 3 to 17 days from the time the first infusion was administered. In some embodiments, the second infusion is administered from about 6 to 16 days from the time the first infusion was administered. In some embodiments, the second infusion is administered from about 13 to 16 days from the time the first infusion was administered. In some embodiments, the second IV infusion is administered 14 days from the time the first IV infusion was administered. In some embodiments, the second IV infusion is administered two weeks from the time the first IV infusion was administered. In some embodiments the terms “14 days” and “2 weeks” are used interchangeably. In some embodiments, the initial anti-CD20 antibody dose comprises a single infusion of anti-CD20 antibody, wherein the single infusion of anti-CD20 antibody is about 1.8 grams. In some embodiments, the second anti-CD20 antibody dose comprises a single infusion of anti-CD20 antibody, wherein the single infusion of anti-CD20 antibody is about 1.8 grams. In some embodiments, the second dose is provided to the patient no sooner than about 20 weeks after the first dose.

In some embodiments, the method comprises providing a third anti-CD20 antibody dose. In some embodiments, the third anti-CD20 antibody dose is provided about 24 weeks from the second dose. In some embodiments, the method comprises providing a third anti-CD20 antibody dose. In some embodiments, the third anti-CD20 antibody dose is provided about 6 months from the second dose. In some embodiments, the third dose is provided to the patient no sooner than 22 weeks after the second dose. In some embodiments, the method further comprises providing a fourth anti-CD20 antibody dose. In some embodiment, the fourth anti-CD20 antibody dose is provided about 24 weeks from the third dose. In some embodiment, the fourth anti-CD20 antibody dose is provided about 6 months from the third dose. In some embodiments, the fourth dose is provided to the patient no sooner than 22 weeks after the third dose. In some embodiments, the method further comprises providing a fifth anti-CD20 antibody dose. In some embodiments, the fifth anti-CD20 antibody dose is provided about 24 weeks from the fourth dose. In some embodiments, the fifth anti-CD20 antibody dose is provided about 6 months from the fourth dose. In some embodiments, the fifth dose is provided to the patient no sooner than 22 weeks after the fourth dose. In some embodiments, subsequent anti-CD20 antibody doses following the fifth anti-CD20 antibody dose are administered at intervals of about 24 weeks. In some embodiments, subsequent anti-CD20 antibody doses following the fifth anti-CD20 antibody dose are administered at intervals of about 6 months. In some embodiments, each subsequent dose of anti-CD20 antibody following the fifth dose is provided to the patient no sooner than 22 weeks following the previous dose of anti-CD20 antibody. In some embodiments, at least 6 doses of anti-CD20 antibody are administered.

In some embodiments, the anti-CD20 antibody comprises a light chain comprising the amino acid sequence of SEQ ID NO: 9 and a heavy chain comprising the amino acid sequence of SEQ ID NO: 11. In some embodiments, the anti-CD20 antibody is ocrelizumab (CAS Registry No. 637334-45-3).

In one embodiment, the patient has never been previously treated with drug(s), such as immunosuppressive agent(s), to treat the multiple sclerosis and/or has never been previously treated with an antibody to a B-cell surface marker (e.g. never previously treated with a CD20 antibody).

In certain embodiments, the patient is premedicated prior to infusion with the anti-CD20 antibody. In certain embodiments, the patient is premedicated with methylprednisolone (or an equivalent) approximately 30 minutes prior to each infusion of anti-CD20 antibody. In certain embodiments, the patient is premedicated with 100 mg IV methylprednisolone (or an equivalent) approximately 30 minutes prior to each infusion of anti-CD20 antibody. In certain embodiments, the patient is additionally (or alternatively) premedicated with an antihistaminic drug (e.g. diphenhydramine) approximately 30-60 minutes before each infusion of anti-CD20 antibody. In certain embodiments, the patient is additionally (or alternatively) premedicated with an antipyretic (e.g. acetaminophen/paracetamol).

While the CD20 antibody may be the only drug administered to the patient to treat the multiple sclerosis, one may optionally administer a second medicament, e.g., a second multiple sclerosis disease modifying agent (DMT), such as a cytotoxic agent, chemotherapeutic agent, immunosuppressive agent, cytokine, cytokine antagonist or antibody, growth factor, hormone, integrin, integrin antagonist or antibody (e.g. an LFA-1 antibody, or an alpha 4 integrin antibody such as natalizumab (TYSABRI®) available from Biogen Idec/Elan Pharmaceuticals, Inc) etc., with the antibody that binds a B cell surface marker (e.g. with the CD20 antibody).

In some embodiments of combination therapy, the antibody is combined with an interferon class drug such as IFN-beta-1a (REBIF® and AVONEX®) or IFN-beta-1b (BETASERON®); an oligopeptide such a glatiramer acetate (COPAXONE®); a cytotoxic agent such as mitoxantrone (NOVANTRONE®), methotrexate, cyclophosphamide, chlorambucil, azathioprine; intravenous immunoglobulin (gamma globulin); lymphocyte-depleting therapy (e.g., mitoxantrone, cyclophosphamide, alemtuzumab (Campath®, LEMTRADA™), anti-CD4, cladribine, total body irradiation, bone marrow transplantation); corticosteroid (e.g. methylprednisolone, prednisone, dexamethasone, or glucorticoid), including systemic corticosteroid therapy; non-lymphocyte-depleting immunosuppressive therapy (e.g., mycophenolate mofetil (MMF) or cyclosporine); cholesterol-lowering drug of the “statin” class, which includes cerivastatin (BAYCOL®), fluvastatin (LESCOL®), atorvastatin (LIPITOR®), lovastatin (MEVACOR®), pravastatin (PRAVACHOL®), Simvastatin (ZOCOR®); estradiol; testosterone (optionally at elevated dosages; Stuve et al. Neurology 8:290-301 (2002)); hormone replacement therapy; treatment for symptoms secondary or related to MS (e.g., spasticity, incontinence, pain, fatigue); a TNF inhibitor; disease-modifying anti-rheumatic drug (DMARD); non-steroidal anti-inflammatory drug (NSAID); plasmapheresis; levothyroxine; cyclosporin A; somatastatin analogue; cytokine or cytokine receptor antagonist; anti-metabolite; immunosuppressive agent; rehabilitative surgery; radioiodine; thyroidectomy; another B-cell surface antagonist/antibody; etc.

The second medicament is administered with the initial anti-CD20 antibody dose and/or later doses of the CD20 antibody, such combined administration includes co-administration, using separate formulations or a single pharmaceutical formulation, and consecutive administration in either order, wherein preferably there is a time period while both (or all) active agents simultaneously exert their biological activities.

In some embodiments, the anti-CD20 antibody is the only medicament administered to the patient to treat multiple sclerosis. In some embodiments, the anti-CD20 antibody is the only disease modifying therapy (DMT) administered to the patient to treat multiple sclerosis. For example, in some embodiments, the anti-CD20 antibody is administered in combination with one or more of: methylprednisolone (or equivalent); an antihistamine (e.g., diphenhydramine or equivalent); an analgesic (e.g., acetaminophen); and an antipyretic.

### Relapsing Multiple Sclerosis (RMS)

In some embodiments, the multiple sclerosis is relapsing multiple sclerosis (RMS). In some embodiments, the patient has been diagnosed RMS according to the criteria described in Thompson et al. (2018) Lancet Neurol. 17:162-73. In some embodiments, the patient has RMS, and treatment results in a reduced risk of 12-week composite disability progression (cCDP). In some embodiments, a reduced risk of 12-week cCDP is measured as an increase in the time to onset of cCDP sustained for at least 12 weeks. In some embodiments, time to onset of cCDP refers to the first occurrence of a confirmed progression event according to one of the following three criteria: (i) confirmed disability progression (CDP); (ii) a sustained increase of 20% in Timed 25-Foot Walk Test (T25FWT) score as compared to the T25FWT score at the start of treatment or just prior to the start of treatment (e.g., within any one of 6, 5, 4, 3, 2, or 1 months or any one of 4, 3, 2, or 1 weeks or within 7, 6, 5, 4, 3, 2, or 1 days before the start of treatment); or (iii) a sustained increase of 20% in 9-Hole Peg Test (9-HPT) score as compared to the 9-HPT score at or just prior to the start of treatment (e.g., within any one of 6, 5, 4, 3, 2, or 1 months or any one of 4, 3, 2, or 1 weeks or within 7, 6, 5, 4, 3, 2, or 1 days before the start of treatment). In some embodiment CDP refers to a sustained increase in EDSS score of 1.0 point in a patient with an EDSS score of 5.5 at or just prior to the start of treatment, or a sustained increase in 0.5 points in a patient with an EDSS score of >5.5 at or just prior to the start of treatment.

The EDSS is a commonly used measure for quantifying changes in the disability level of patients with MS over time. The EDSS is a disability scale that ranges in 0.5-point steps from 0 (normal) to 10.0 (death) (see Kurtzke (1983) Neurol 1983; 33:1444-52; and Kappos (2011) Neurology, University Hospital Basel, Switzerland: Neurostatus Scoring Definitions). The EDSS is based on a standard neurological examination, incorporating functional systems (visual, brainstem, pyramidal, cerebellar, sensory, bowel and bladder, and cerebral [or mental]) that are rated and then scored as a FSS (functional system score), and ambulation, which is scored as ambulation score. Each FSS is an ordinal clinical rating scale ranging from 0 to 5 or 6 and an ambulation score that is rated from 0 to 16. These ratings are then used in conjunction with observations, as well as information, concerning ambulation and use of assistive devices to determine the total EDSS score. In some embodiments, the EDSS is administered according to the criteria and calculated according to the algorithm described in D'Souza M, Yaldizli Ö, John R, et al. Neurostatus e-Scoring improves consistency of Expanded Disability Status Scale assessments: A proof of concept study. Mult Scler Houndmills Basingstoke Engl. 2017; (4):597-603.

The T25FWT test is a performance measure used to assess walking speed based on a timed 25-foot walk. Typically, the patient is directed to start at one end of a clearly marked 25-foot course and is instructed to walk 25 feet as quickly and safely as possible. A qualified individual (e.g., a physician, neurologist, etc.) times the patient from the start of the walk to the end of the 25 feet. In some embodiments, the task is immediately administered again by having the patient walk back the same distance. In some embodiments, the score for the T25FWT is the average of the two completed trials. In some embodiments, the use of assistive devices (i.e., cane or wheelchair) is permitted when performing the T25FWT. In some embodiments, the same assistive device is used each time the patient performs the T25WT. A 20% change from baseline (e.g., at or just prior to the start of treatment) of the averaged T25FWT is typically considered clinically meaningful (www(dot)ema(dot)europa(dot)eu/en/documents/scientificguideline/draft-qualification-opinion-multiple-sclerosis-clinical-outcomeassessment-mscoa_en(dot)pdf and Hobart J, Blight A R, Goodman, A, et al. Timed 25-foot walk: direct evidence that improving 20% or greater is clinically meaningful in MS. Neurology 2013; 80(16):1509-17). In some embodiments, the T25FWT is administered as described in the MSFC Administration and Scoring Manual (see www(dot)nationalmssociety(dot)org/nationalmssociety/media/msnationalfiles/brochures/10-2-3-31-msfc_manual_and_forms(dot)pdf).

The 9-HPT is a performance measure used to assess upper extremity (arm and hand) function (Goodkin et al. (1988) Arch Phys Med Rehabil. 69:850-54; Fischer (1999) Mult Scler 5:244-50). Typically, the test comprises a container containing nine pegs and a wood or plastic block containing nine empty holes. The patient is to pick up each of the nine pegs one at a time and as quickly as possible place them in the nine holes. Once all the pegs are in the holes, the patient is to remove them again one at a time as quickly as possible and replace them into the container. The total time to complete the test is typically recorded, e.g., by a qualified individual (e.g., physician, neurologist, etc.). In some embodiments, both the dominant and non-dominant hands are tested twice (two consecutive trials of the dominant hand, followed immediately by two consecutive trials of the non-dominant hand). A 20% change from baseline is typically considered clinically meaningful (Feys et al. (2017) Multiple Sclerosis Journal 23(5):711-20).

In some embodiments, the patient has RMS, and treatment results (or, in addition to the efficacy measures discussed above, further results) in one or more of: (a) increase in time to onset of 24-week cCDP (i.e., cCDP that is sustained for at least 24 weeks); (b) increase in time to onset of 12-week confirmed disability progression (CDP) (i.e., CDP that is sustained for at least 12 weeks); (c) increase in time to onset of 24-week CDP (i.e., CDP that is sustained for at least 24 weeks); (d) increase in time to ≥20% increase in 12-week confirmed T25FWT (i.e., a ≥20% increase in T25FWT score that is sustained for at least 12 weeks); (e) increase in time to ≥20% increase in 24-week confirmed T25FWT (i.e., a ≥20% increase in T25FWT score that is sustained for at least 24 weeks); (f) decrease in the percent change in total brain volume (e.g., decrease in rate of brain volume loss) after 24, 48, 72, 96, and 120 weeks of treatment; and (g) increase in time to 12-week confirmed 4-point worsening in Symbol Digital Modality Test (SDMT) (i.e., a 4-point worsening in SDMT that is sustained for 12 weeks).

The SDMT is a performance measure that has demonstrated sensitivity in detecting not only the presence of cognitive impairment but also changes in cognitive functioning over time and in response to treatment (Smith A. Symbol digit modalities test: manual. Los Angeles: Western Psychological Services, 1982). The SDMT is recognized in the art as being particularly sensitive to slowed processing of information that is commonly seen in MS (Benedict et al (2017) Mult Scler 23(5):721-33). Briefly, using a reference key, the patient has 90 seconds to pair specific numbers with given geometric figures. Responses are collected orally. A four-point change from baseline is typically considered clinically meaningful.

In some embodiments, the patient has RMS, and treatment results (or, in addition to any one or more of the efficacy measures discussed above, further results) in one or more of: (A) reduction or no change in Expanded Disability Status Scare (EDSS) score; (B) increase in time to ≥20% increase in 12-week confirmed 9-hole peg test (9-HPT) (i.e., ≥20% increase in 9-HPT that is sustained for 12 weeks); (C) increase in time to ≥20% increase in 24-week confirmed 9-HPT (i.e., ≥20% increase in 9-HPT that is sustained for 24 weeks); (D) increase in time to onset of cCDP 12 and progression in cCDP individual components independent of relapses; (E) reduction in new T1-hypointense lesions; (F) reduction in volume of T1-hypointense lesions; (G) reduction in spinal cord volume loss; (H) reduction in annualized relapse rate (ARR); (I) increase in time to onset of 12-week confirmed relapse-associated worsening (RAW) and individual components; (J) reduction in number of new T2 lesions and enlarging T2 lesions over treatment period; and (K) reduction in number of T1 Gd⁺ staining lesions over treatment period. In some embodiments, ARR refers to the number of relapses a patient with RMS has in one year. In some embodiments, ARR refers to the average number of relapses a group of patients in a clinical study have in one year. In some embodiments, a relapse is defined as the occurrence of new or worsening neurological symptoms attributable to MS and immediately preceded by a relatively stable or improving neurological state of least 30 days. In some embodiments, the symptoms persist for >24 hours and are not attributable to confounding clinical factors (e.g., fever, infection, injury, adverse reactions to concomitant medications). In some embodiments, the new or worsening neurological symptoms are accompanied by objective neurological worsening consistent with an increase of at least one of the following: (a) half a step (0.5 point) on the EDSS; (b) two points on one of the selected FSS (as listed in (c)); and (c) one point on two or more of the following selected FSS: pyramidal, ambulation, cerebellar, brainstem, sensory, or visual. In some embodiments, RAW is refers to a confirmed disability accumulation (CDA) with the initial disability increase occurring 90 or fewer days after the onset of a relapse. In some embodiments, CDA is defined as disability increase from start of treatment as measured by EDSS (increase of ≥1.0 points if baseline EDSS ≤5.5 points or an ≥0.5-point increase if baseline EDSS >5.5 points). In some embodiments, RAW refers to the onset of confirmed worsening by 1.0 point or more in EDSS score within 180 days of a relapse.

In some embodiments, the patient has been diagnosed with RMS in accordance with the revised McDonald Criteria 2017 (Thompson A J, Banwell B L, Barkhof F, et al. Diagnosis of multiple sclerosis: 2017 revisions of the McDonald criteria. Lancet Neurol 2018; 17:162-73). In some embodiments, the patient with RMS has not received prior treatment with an anti-CD20 antibody. In some embodiments, the patient with RMS has received prior treatment with an anti-CD20 antibody, and the last dose of anti-CD20 antibody was more than about two years prior to the start of treatment according to a method herein. In some embodiments, the patient with RMS has received prior treatment with an anti-CD20 antibody, and the patient has normal B-cell count. In some embodiments, the patient with RMS has received prior treatment with an anti-CD20 antibody, and the treatment was not discontinued due to lack of efficacy and/or adverse event. In some embodiments, the patient with RMS received prior treatment with rituximab, ocrelizumab, obinutuzumab, veltuzumab, tositumomab, ibritumomab, ofatumumab. In some embodiments, the patient with RMS has not received prior treatment with mitoxantrone, cladribine, atacicept, and/or alemtuzumab.

Progressive Multiple Sclerosis (PPMS)

In some embodiments, the multiple sclerosis is primary progressive multiple sclerosis (PPMS). In some embodiments, the patient has been diagnosed PPMS according to the criteria described in Thompson et al. (2018) Lancet Neurol. 17:162-73. In some embodiments, the patient has PPMS, and treatment results in a reduced risk of 12-week composite disability progression (cCDP).

In some embodiments, the patient has PPMS, and treatment results (or, in addition to the efficacy measures discussed above, further results) in one or more of: (a) increase in time to onset of 24-week cCDP; (b) increase in time to onset of 12-week confirmed disability progression (CDP); (c) increase in time to onset of 24-week CDP; (d) increase in time to >20% increase in 12-week confirmed timed 25 foot walk test (T25FWT); (e) increase in time to >20% increase in 24-week confirmed T25FWT; (f) increase in time to >20% increase in 12-week confirmed 9-hole peg test (9-HPT); (g) increase in time to >20% increase in 24-week confirmed 9-HPT; (h) decrease in loss of total brain volume during over treatment period following second ant-CD20 antibody dose; and (i) increase in time to 12-week confirmed 4-point worsening in Symbol Digital Modality Test (SDMT).

In some embodiments, the patient has PPMS, and treatment results (or, in addition to the efficacy measures discussed above, further results) in one or more of: (A) a reduction or no change in Expanded Disability Status Scare (EDSS) score; (B) reduction in new T1-hypointense lesions; (C) reduction in volume of T1-hypointense lesions; (D) reduction in spinal cord volume loss; (E) reduction in number of new T2 lesions and enlarging T2 lesions over treatment period; and (F) reduction in number of T1 Gd⁺ staining lesions over treatment period.

In some embodiments, the patient with PPMS has not received prior treatment with an anti-CD20 antibody. In some embodiments, the patient with PPMS has received prior treatment with an anti-CD20 antibody, and the last dose of anti-CD20 antibody was more than about two years prior to the start of treatment according to a method herein. In some embodiments, the patient with PPMS has received prior treatment with an anti-CD20 antibody, and the patient has normal B-cell count. In some embodiments, the patient with PPMS has received prior treatment with an anti-CD20 antibody, and the treatment was not discontinued due to lack of efficacy and/or adverse event. In some embodiments, the patient with PPMS has received prior treatment with ocrelizumab. In some embodiments, the patient with RMS received prior treatment with rituximab, ocrelizumab, obinutuzumab, veltuzumab, tositumomab, ibritumomab, ofatumumab. In some embodiments, the patient with RMS has not received prior treatment with mitoxantrone, cladribine, atacicept, and/or alemtuzumab.

In some embodiments, the patient has been diagnosed with PPMS in accordance with the revised McDonald Criteria 2017 (Thompson A J, Banwell B L, Barkhof F, et al. Diagnosis of multiple sclerosis: 2017 revisions of the McDonald criteria. Lancet Neurol 2018; 17:162-73). In some embodiments, the patient has an EDSS score between 3 to 6.5, inclusive, at the start of treatment (e.g., prior to the first dose of anti-CD20 antibody). In some embodiments, the patient has a score of ≥2.0 on the Functional Systems (FS) scale for the pyramidal system that is due to lower extremity findings. In some embodiments, the patient has a disease duration of less than about 15 years from onset of MS symptoms with an EDSS score of >5.0 at the start of treatment (e.g., prior to the first dose of anti-CD20 antibody). In some embodiments, the patient has a disease duration of less than about 10 years from the onset of MS symptoms with an EDSS score at screening of ≤5.0. IN some embodiments, the patient has documented evidence of the presence of cerebrospinal fluid-specific oligoclonal bands.

Antibodies and their Production

The methods and articles of manufacture of the present invention use, or incorporate, an antibody that binds to a B-cell surface marker, especially one that binds to CD20. Accordingly, methods for generating such antibodies will be described here.

In some embodiments, the anti-CD20 antibody used in the methods described here is produced by a method comprising expressing a nucleic acid encoding a humanized antibody comprising the heavy and light chain amino acid sequences of SEQ ID NO:14 or 13, respectively, in a host cell, and recovering the humanized antibody or an antigen-binding fragment thereof expressed in the host cell. In some embodiments, the host cell is a mammalian cell (e.g., a CHO cell), an insect cell, or a plant cell. In some embodiments the host cell is a bacterial cell. Methods of producing an anti-CD20 are described in further detail in, e.g., U.S. Pat. No. 7,799,900.

The B cell surface marker to be used for production of, or screening for, antibodies may be, e.g., a soluble form of the marker or a portion thereof, containing the desired epitope. Alternatively, or additionally, cells expressing the marker at their cell surface can be used to generate, or screen for, antibodies. Other forms of the B cell surface marker useful for generating antibodies will be apparent to those skilled in the art.

A description follows as to exemplary techniques for the production of the antibodies used in accordance with the present invention.

### Humanized Antibodies

Methods for humanizing non-human antibodies have been described in the art. In some embodiments, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, in some embodiments of the methods, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

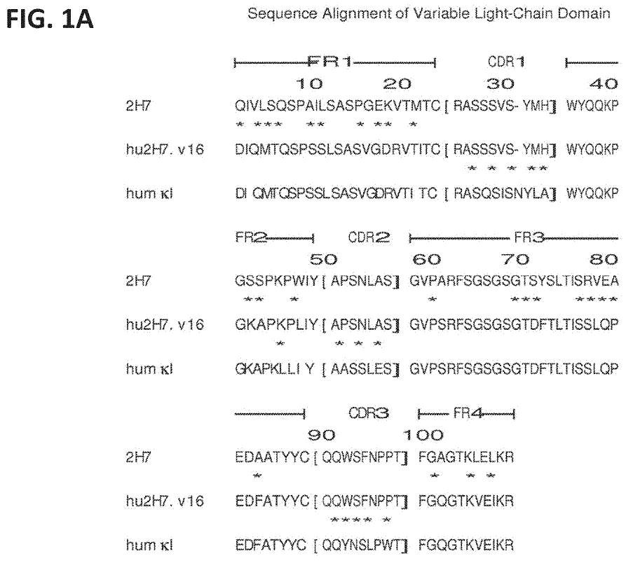

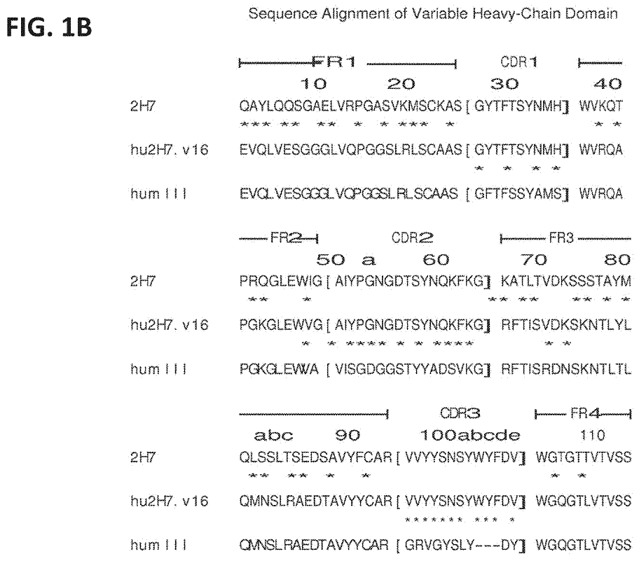

In some embodiments, the humanized anti-CD20 antibody is ocrelizumab. Ocrelizumab comprises the six of CDR sequences as shown in FIGS. 1A and 1B:

CDR L1 sequence
(SEQ ID NO: 1)
RASSSVSYMH
(FIG. 1A),
CDR L2 sequence
(SEQ ID NO: 2)
APSNLAS
(FIG. 1A),
CDR L3 sequence
(SEQ ID NO: 3)
QQWSFNPPT
(FIG. 1A),
CDR H1 sequence
(SEQ ID NO: 4)
GYTFTSYNMH
(FIG. 1B),
CDR H2 sequence
(SEQ ID NO: 5)
AIYPGNGDTSYNQKFKG
(FIG. 1B),
and
CDR H3 sequence
(SEQ ID NO: 6)
VVYYSNSYWYFDV
(FIG. 1B).
Ocrelizumab comprises the variable
light chain sequence:
(SEQ ID NO: 7)
DIQMTQSPSSLSASVGDRVTITCRASSSVSYMHWY
QQKPGKAPKPLIYAPSNLASGVPSRFSGSGSGTDF
TLTISSLQPEDFATYYCQQWSFNPPTFGQGTKVEI
KR;
and the variable heavy chain sequence:
(SEQ ID NO: 8)
EVQLVESGGGLVQPGGSLRLSCAASGYTFTSYNMH
WVRQAPGKGLEWVGAIYPGNGDTSYNQKFKGRFTI
SVDKSKNTLYLQMNSLRAEDTAVYYCARVVYYSNS
YWYFDVWGQGTLVTVSS.
Ocrelizumab comprises the light
chain amino acid sequence:
(SEQ ID NO: 9)
DIQMTQSPSSLSASVGDRVTITCRASSSVSYMHWY
QQKPGKAPKPLIYAPSNLASGVPSRFSGSGSGTDF
TLTISSLQPEDFATYYCQQWSFNPPTFGQGTKVEI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC;
and the heavy chain amino acid sequence:
(SEQ ID NO: 10)
EVQLVESGGGLVQPGGSLRLSCAASGYTFTSYNMH
WVRQAPGKGLEWVGAIYPGNGDTSYNQKFKGRFTI
SVDKSKNTLYLQMNSLRAEDTAVYYCARVVYYSNS
YWYFDVWGQGTLVTVSSASTKGPSVFPLAPSSKST
SGGTAALGCLVKDYFPEPVTVSWNSGALTSGVHTF
PAVLQSSGLYSLSSVVTVPSSSLGTQTYICNVNHK
PSNTKVDKKVEPKSCDKTHTCPPCPAPELLGGPSV
FLFPPKPKDTLMISRTPEVTCVVVDVSHEDPEVKF
NWYVDGVEVHNAKTKPREEQYNSTYRVVSVLTVLH
QDWLNGKEYKCKVSNKALPAPIEKTISKAKGQPRE
PQVYTLPPSREEMTKNQVSLTCLVKGFYPSDIAVE
WESNGQPENNYKTTPPVLDSDGSFFLYSKLTVDKS
RWQQGNVFSCSVMHEALHNHYTQKSLSLSPGK
or the heavy chain amino acid sequence:
(SEQ ID NO: 11)
EVQLVESGGGLVQPGGSLRLSCAASGYTFTSYNMH
WVRQAPGKGLEWVGAIYPGNGDTSYNQKFKGRFTI
SVDKSKNTLYLQMNSLRAEDTAVYYCARVVYYSNS
YWYFDVWGQGTLVTVSSASTKGPSVFPLAPSSKST
SGGTAALGCLVKDYFPEPVTVSWNSGALTSGVHTF
PAVLQSSGLYSLSSVVTVPSSSLGTQTYICNVNHK
PSNTKVDKKVEPKSCDKTHTCPPCPAPELLGGPSV
FLFPPKPKDTLMISRTPEVTCVVVDVSHEDPEVKF
NWYVDGVEVHNAKTKPREEQYNSTYRVVSVLTVLH
QDWLNGKEYKCKVSNKALPAPIEKTISKAKGQPRE
PQVYTLPPSREEMTKNQVSLTCLVKGFYPSDIAVE
WESNGQPENNYKTTPPVLDSDGSFFLYSKLTVDKS
RWQQGNVFSCSVMHEALHNHYTQKSLSLSPG.

In some embodiments, the amino acid K at C-terminus of the heavy chain is removed.

### Pharmaceutical Formulations

Therapeutic formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Lyophilized formulations adapted for subcutaneous administration are described in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

Crystalized forms of the antibody or antibody are also contemplated. See, for example, US 2002/0136719A1 (Shenoy et al.).

The formulation herein may also contain more than one active compound as necessary for the particular indication being treated, in some embodiments, those with complementary activities that do not adversely affect each other. For example, it may be desirable to further provide a cytotoxic agent; chemotherapeutic agent; immunosuppressive agent; cytokine; cytokine antagonist or antibody; growth factor; hormone; integrin; integrin antagonist or antibody (e.g. an LFA-1 antibody, or an alpha 4 integrin antibody such as natalizumab/TYSABRI®) available from Biogen Idec/Elan Pharmaceuticals, Inc.); interferon class drug such as IFN-beta-1a (REBIF® and AVONEX®) or IFN-beta-1b (BETASERON®); an oligopeptide such a glatiramer acetate (COPAXONE®); a cytotoxic agent such as mitoxantrone (NOVANTRONE®), methotrexate, cyclophosphamide, chlorambucil, or azathioprine; intravenous immunoglobulin (gamma globulin); lymphocyte-depleting drug (e.g., mitoxantrone, cyclophosphamide, Campath, anti-CD4, or cladribine); non-lymphocyte-depleting immunosuppressive drug (e.g., mycophenolate mofetil (MMF) or cyclosporine); cholesterol-lowering drug of the “statin” class; estradiol; testosterone; hormone replacement therapy; drug that treats symptoms secondary or related to MS (e.g., spasticity, incontinence, pain, fatigue); a TNF inhibitor; disease-modifying anti-rheumatic drug (DMARD); non-steroidal anti-inflammatory drug (NSAID); corticosteroid (e.g. methylprednisolone, prednisone, dexamethasone, or glucorticoid); levothyroxine; cyclosporin A; somatastatin analogue; cytokine antagonist; anti-metabolite; immunosuppressive agent; integrin antagonist or antibody (e.g. an LFA-1 antibody, such as efalizumab or an alpha 4 integrin antibody such as natalizumab); or another B-cell surface antagonist/antibody; etc. in the formulation. The type and effective amounts of such other agents depend, for example, on the amount of antibody present in the formulation, the type of multiple sclerosis being treated, and clinical parameters of the patients. These are generally used in the same dosages and with administration routes as used hereinbefore or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

In some embodiments, the formulation comprises one or more of the group consisting of a histidine buffer, trehalose, sucrose, and polysorbate 20. In some embodiments, the histidine buffer is a histidine-acetate buffer, pH 6.0. Examples of formulations suitable for the administration of the anti-CD20 antibody are found in Andya et al., US2006/0088523, which is incorporated by reference in its entirety with respect to formulations.

Exemplary anti-CD20 antibody formulations are described in Andya et al., US2006/0088523 and WO98/56418, which are incorporated by reference in its entirety. In some embodiments, formulation is a liquid multidose formulation comprising the anti-CD20 antibody at 40 mg/mL, 25 mM acetate, 150 mM trehalose, 0.9% benzyl alcohol, 0.02% polysorbate 20 at pH 5.0 that has a minimum shelf life of two years storage at 2-8° C. In some embodiments, anti-CD20 formulation of interest comprises 10 mg/mL antibody in 9.0 mg/mL sodium chloride, 7.35 mg/mL sodium citrate dihydrate, 0.7 mg/mL polysorbate 80, and Sterile Water for Injection, pH 6.5. In some embodiments, the anti-CD20 antibody is in an aqueous pharmaceutical formulation comprising 10-30 mM sodium acetate from about pH 4.8 to about pH 5.5, preferably at pH5.5, polysorbate as a surfactant in a an amount of about 0.01-0.1% v/v, trehalose at an amount of about 2-10% w/v, and benzyl alcohol as a preservative (U.S. Pat. No. 6,171,586, which is incorporated by reference in its entirety). Lyophilized formulations adapted for subcutaneous administration are described in WO97/04801, which is incorporated by reference in its entirety. Such lyophilized formulations may be reconstituted with a suitable diluent to a high protein concentration and the reconstituted formulation may be administered subcutaneously to the mammal to be treated herein.

In some embodiments, the humanized 2H7 variants formulation is antibody at 12-14 mg/mL in 10 mM histidine, 6% sucrose, 0.02% polysorbate 20, pH 5.8. In a specific embodiment, 2H7 variants and in particular 2H7.v16 is formulated at 20 mg/mL antibody in 10 mM histidine sulfate, 60 mg/ml sucrose., 0.2 mg/ml polysorbate 20, and Sterile Water for Injection, at pH5.8. In a specific embodiment, one IV formulation of humanized 2H7 v16 is: 30 mg/ml antibody in 20 mM sodium acetate, 4% trehalose dihydrate, 0.02% polysorbate 20 (Tween 20™), pH 5.3. In some embodiments, the humanized 2H7.v511 variant formulation is 15-30 mg/ml antibody, preferably 20 mg/mL antibody, in 10 mM histidine sulfate, 60 mg/ml sucrose (6%), 0.2 mg/ml polysorbate 20 (0.02%), and Sterile Water for Injection, at pH5.8. In yet another embodiment, the formulation for 2H7 variants and in particular 2H7.v511 is 20 mg/ml 2H7, 20 mM sodium acetate, 4% trehalose dihydrate, 0.02% polysorbate 20, pH 5.5, for intravenous administration. In some embodiments, 2H7.v 114 formulation is antibody at 15-25 mg/ml, preferably 20 mg/ml, in 20 mM Sodium Acetate, 240 mM (8%) trehalose dihydrate, 0.02% Polysorbate 20, pH 5.3. In some embodiments, the anti-CD20 antibody (e.g., 2H7.v16) is in a formulation comprising 30 mg/mL antibody, 20 mM Sodium Acetate, 106 mM Trehalose, 0.02% polysorbate 20, and pH 5.3. The liquid formulation containing the antibody may be in 300 mg/vial, and may be stored at 2-8° C., protected from light. In some embodiments, prior to administration, the antibody formulation is diluted with normal saline (0.9% Sodium Chloride) in an IV bag for administration by infusion.

### Articles of Manufacture and Kits

The invention further provides articles of manufacture or kits (such as kits-of parts) containing materials useful for the treatment of multiple sclerosis (e.g., relapsing multiple sclerosis or primary progressive multiple sclerosis) described herein. In some embodiments, the article of manufacture comprising, packaged together, a pharmaceutical composition comprising an anti-CD20 antibody and a pharmaceutically acceptable carrier and a label denoting that the anti-CD20 antibody or pharmaceutical composition is indicated for treating patients with multiple sclerosis (e.g., RMS or PPMS) according to a method described herein.

In some embodiments, the article of manufacture or kit comprises, packaged together, a pharmaceutical composition comprising an anti-CD20 antibody and a pharmaceutically acceptable carrier and a label denoting the anti-CD20 antibody or pharmaceutical composition is indicated for treating patients with multiple sclerosis and suppresses disability progression in patients having multiple sclerosis. In some embodiments, the article of manufacture or kit comprises, packaged together, a pharmaceutical composition comprising an anti-CD20 antibody and a pharmaceutically acceptable carrier and a label denoting the anti-CD20 antibody or pharmaceutical composition is indicated for treating patients with multiple sclerosis (e.g., RMS or PPMS). In some embodiments, the label provides instructions for administering an effective amount of an anti-CD20 antibody to the patient to provide an initial anti-CD20 antibody dose of about 1.2 grams followed by a second anti-CD20 antibody dose of about 1.2 grams, the second dose not being provided until from about 24 weeks or 6 months from the initial dose, wherein the patient weighs less than about 75 kg at the time of the first anti-CD20 antibody dose. In some embodiments, the label states that the initial anti-CD20 antibody dose comprises a first intravenous (IV) infusion and a second IV infusion of anti-CD20 antibody, wherein the first IV infusion and second IV infusion of anti-CD20 antibody are each about 0.6 grams. In some embodiments, the label states that the initial anti-CD20 antibody dose comprises a single IV infusion of anti-CD20 antibody, wherein the single IV infusion of anti-CD20 antibody is about 1.2 grams. In some embodiments, the label states that the second anti-CD20 dose comprises a single IV infusion of anti-CD20 antibody, wherein the single IV fusion of anti-CD20 antibody is about 1.2 grams. In some embodiments, the label provides instructions for administering an effective amount of an anti-CD20 antibody to the patient to provide an initial anti-CD20 antibody dose of about 1.8 grams followed by a second anti-CD20 antibody dose of about 1.8 grams, the second dose not being provided until from about 24 weeks or 6 months from the initial dose, wherein the patient weighs about 75 kg or more at the time of the first anti-CD20 antibody dose. In some embodiments, the label states that the initial anti-CD20 antibody dose comprises a first intravenous (IV) infusion and a second IV infusion of anti-CD20 antibody, wherein the first IV infusion and second IV infusion of anti-CD20 antibody are each about 0.9 grams. In some embodiments, the label states that the initial anti-CD20 antibody dose comprises a single IV infusion of anti-CD20 antibody, wherein the single IV infusion of anti-CD20 antibody is about 1.8 grams. In some embodiments, the label states that the second anti-CD20 dose comprises a single IV infusion of anti-CD20 antibody, wherein the single IV fusion of anti-CD20 antibody is about 1.8 grams. In some embodiments, the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region. In some embodiments, the anti-CD20 antibody comprises a light chain comprising the amino acid sequence of SEQ ID NO: 9 and a heavy chain comprising the amino acid sequence of SEQ ID NO: 11.

In some embodiments, label denotes that the anti-CD20 antibody or pharmaceutical composition is indicated for treating patients with relapsing multiple sclerosis, and that treatment results in reduced risk of 12-week composite confirmed disability progression (cCDP 12). Additionally or alternatively, in some embodiments, label denotes that the anti-CD20 antibody or pharmaceutical composition is indicated for treating patients with relapsing multiple sclerosis, and that treatment results in one or more of: (a) increase in time to onset of 24-week cCDP; (b) increase in time to onset of 12-week confirmed disability progression (CDP); (c) increase in time to onset of 24-week CDP; (d) increase in time to ≥20% increase in 12-week confirmed timed 25 foot walk test (T25FWT); (e) increase in time to ≥20% increase in 24-week confirmed T25FWT; (f) decrease in the percent change in total brain volume after 24, 48, 72, 96, and 120 weeks of treatment; and (g) increase in time to 12-week confirmed 4-point worsening in Symbol Digital Modality Test (SDMT). Additionally or alternatively, in some embodiments, label denotes that the anti-CD20 antibody or pharmaceutical composition is indicated for treating patients with relapsing multiple sclerosis, and that treatment results in one or more of: (A) reduction or no change in Expanded Disability Status Scare (EDSS) score; (B) increase in time to ≥20% increase in 12-week confirmed 9-hole peg test (9-HPT); (C) increase in time to ≥20% increase in 24-week confirmed 9-HPT; (D) increase in time to onset of cCDP 12 and progression in cCDP individual components independent of relapses; (E) reduction in new T1-hypointense lesions; (F) reduction in volume of T1-hypointense lesions; (G) reduction in spinal cord volume loss; (H) reduction in annualized relapse rate (ARR); (I) increase in time to onset of 12-week confirmed relapse-associated worsening (RAW) and individual components; (J) reduction in number of new or enlarging T2 lesions over treatment period; and (K) reduction in number of T1 Gd⁺ staining lesions over treatment period.

In some embodiments, label denotes that the anti-CD20 antibody or pharmaceutical composition is indicated for treating patients with primary progressive multiple sclerosis, and that treatment results in reduced risk of 12-week composite confirmed disability progression (cCDP 12). Additionally or alternatively, in some embodiments, label denotes that the anti-CD20 antibody or pharmaceutical composition is indicated for treating patients with primary progressive multiple sclerosis, and that treatment results in one or more of: (a) increase in time to onset of 24-week cCDP; (b) increase in time to onset of 12-week confirmed disability progression (CDP); (c) increase in time to onset of 24-week CDP; (d) increase in time to ≥20% increase in 12-week confirmed timed 25 foot walk test (T25FWT); (e) increase in time to ≥20% increase in 24-week confirmed T25FWT; (f) increase in time to ≥20% increase in 12-week confirmed 9-hole peg test (9-HPT); (g) increase in time to ≥20% increase in 24-week confirmed 9-HPT; (h) decrease in loss of total brain volume during over treatment period following second ant-CD20 antibody dose; and (i) increase in time to 12-week confirmed 4-point worsening in Symbol Digital Modality Test (SDMT). Additionally or alternatively, in some embodiments, label denotes that the anti-CD20 antibody or pharmaceutical composition is indicated for treating patients with primary progressive multiple sclerosis, and that treatment results in one or more of (A) a reduction or no change in Expanded Disability Status Scare (EDSS) score; (B) reduction in new T1-hypointense lesions; (C) reduction in volume of T1-hypointense lesions; (D) reduction in spinal cord volume loss; (E) reduction in number of new or enlarging T2 lesions over treatment period; and (F) reduction in number of T1 Gd⁺ staining lesions over treatment period.

In certain embodiments, the article of manufacture or kit comprises a container and a label or package insert on or associated with the container. Suitable containers include, for example, bottles, vials, syringes, etc. The containers may be formed from a variety of materials such as glass or plastic. The container holds or contains a composition that is effective for treating the multiple sclerosis and may have a sterile access port (for example the container may be an intravenous solution bag or a vial having a stopper pierceable by a hypodermic injection needle). At least one active agent in the composition is the antibody. In some embodiments, the container comprises between about 0.3 to about 1.5 grams of the anti-CD20 antibody. In some embodiments, the container comprises between about 0.3 to about 2.0 grams of the anti-CD20 antibody.

The label or package insert indicates that the composition is used for treating multiple sclerosis in a patient suffering therefrom with specific guidance regarding dosing amounts and intervals of antibody and any other drug being provided. The article of manufacture may further comprise a second container comprising a pharmaceutically acceptable diluent buffer, such as bacteriostatic water for injection (BWFI), phosphate-buffered saline, Ringer's solution and dextrose solution. The article of manufacture may further include other materials desirable from a commercial and user standpoint, including other buffers, diluents, filters, needles, and syringes.

Optionally, the article of manufacture or kit provided herein further comprises a container comprising an agent other than the antibody for treatment and further comprising instructions on treating the patient with such agent, such agent preferably being a chemotherapeutic agent or immunosuppressive agent, interferon class drug such as IFN-beta-1a (REBIF® and AVONEX®) or IFN-beta-1b (BETASERON®); an oligopeptide such a glatiramer acetate (COPAXONE®); a cytotoxic agent such as mitoxantrone (NOVANTRONE®), methotrexate, cyclophosphamide, chlorambucil, or azathioprine; intravenous immunoglobulin (gamma globulin); lymphocyte-depleting drug (e.g., mitoxantrone, cyclophosphamide, Campath, anti-CD4, or cladribine); non— lymphocyte-depleting immunosuppressive drug (e.g., mycophenolate mofetil (MMF) or cyclosporine); cholesterol-lowering drug of the “statin” class; estradiol; hormone replacement therapy; drug that treats symptoms secondary or related to MS (e.g., spasticity, incontinence, pain, fatigue); a TNF inhibitor; disease-modifying anti-rheumatic drug (DMARD); non-steroidal anti-inflammatory drug (NSAID); corticosteroid (e.g. methylprednisolone, prednisone, dexamethasone, or glucorticoid); levothyroxine; cyclosporin A; somatastatin analogue; cytokine or cytokine receptor antagonist; anti-metabolite; immunosuppressive agent; integrin antagonist or antibody (e.g. an LFA-1 antibody, such as efalizumab or an alpha 4 integrin antibody such as natalizumab); and another B-cell surface marker antibody; etc.

### EXAMPLES

### Example 1: Ocrelizumab in Relapsing Multiple Sclerosis and Primary Progressive Multiple Sclerosis: Pharmacokinetic and Pharmacodynamic Analyses of Three Phase III Clinical Trials

B cells are thought to play an important role in the pathogenesis of MS. Ocrelizumab is a humanized monoclonal antibody that selectively targets CD20-positive B cells, resulting in antibody-dependent cellular cytolysis, antibody-dependent cellular phagocytosis, apoptosis, and/or complement-mediated lysis of the B cells. Ocrelizumab is indicated for treatment of patients with relapsing forms of multiple sclerosis (RMS) or primary progressive multiple sclerosis (PPMS). The pharmacokinetics and pharmacodynamics of ocrelizumab in patients with RMS or PPMS patients were assessed. As discussed in greater detail below, a population pharmacokinetic model was developed based on data from a Phase II study and two Phase III studies of ocrelizumab patients with RMS. Data from a Phase III study of ocrelizumab in patients with PPMS became available after model finalization and was used for external model evaluation. The ocrelizumab serum concentration vs. time course was accurately described by a two-compartment model with time-dependent clearance. Body weight was found to be the main covariate. The area under the concentration—time curve over the dosing interval was estimated to be 26% higher for patients with RMS weighing <60 kg and 21% lower for patients weighing >90 kg when compared with the 60-90 kg group. The terminal half-life of ocrelizumab was estimated as 26 days. The extent of B-cell depletion in blood, as the pharmacodynamic marker, was greater with increasing ocrelizumab exposure. The pharmacokinetics of ocrelizumab was described with pharmacokinetic parameters typical for an immunoglobulin G1 monoclonal antibody, with body weight as the main covariate. The pharmacokinetics and B-cell depletion in blood were comparable across the RMS and PPMS trials, and the extent of B-cell depletion was greater with higher exposure.

### INTRODUCTION

Multiple sclerosis (MS) is the most common chronic inflammatory, demyelinating and neurodegenerative disease of the central nervous system in young adults. It is characterized by symptoms such as visual loss; paresis and spasticity; sensory disturbances and numbness; incoordination; bowel, bladder and sexual dysfunction; fatigue; pain; and cognitive defects (Thomson et al. (2018) Lancet 391: 1622-36; Reich et al. (2018) N Engl J Med. 378: 169-80). MS can be categorized as relapsing or progressive but is largely considered a progressive disease in most patients, regardless of the phenotype (Cree et al. (2019) Ann Neurol. 85: 653-66). Relapsing MS (RMS) begins as an episodic disorder, but can evolve into a condition characterized by progressive neurological disability termed secondary progressive MS (Thomson et al. (2018) Lancet. 391: 1622-36; Reich et al. (2018) N Engl J Med. 378: 169-80; Noseworthy et al. (2000) N Engl J Med. 343: 938-52). Primary progressive multiple sclerosis (PPMS), which accounts for 10-15% of the MS patient population (Miller et al. (2007) Lancet Neurol. 6: 903-12), presents with a disease course that consists mainly of gradual worsening of neurological disability from symptom onset, although relapses may occur (Montalban et al. (2017) N Engl J Med. 376: 209-20).

MS was long thought to be a T-cell-mediated autoimmune disorder, causing inflammatory demyelination and neuronal damage, which slows or prevents nerve signaling (Wekerle (2008) Ann Rheum Dis. 67(suppl 3): iii56-60). Recently B cells have been shown to play an important role in the pathogenesis of MS likely via a number of mechanisms, such as the presentation of autoantigens and costimulatory signals to activate T cells and the secretion of pro-inflammatory cytokines (Gasperi C et al. (2016) Neurodegener Dis Manag. 6: 37-47; Constant (1999) J Immunol. 162: 5695-703; Crawford et al. (2006) J Immunol. 176: 3498-506; Bar-Or et al. (2010) Ann Neurol. 67: 452-61; Duddy et al. (2007) J Immunol. 178: 6092-9.

Ocrelizumab is a recombinant humanized monoclonal antibody that targets CD20-positive B cells (Klein et al. (2013) mAbs 5: 22-33. CD20 is a cell surface antigen found on pre-B cells, mature B cells and memory B cells, but is not expressed on lymphoid stem cells and mature plasma cells. The precise mechanisms by which ocrelizumab exerts its therapeutic clinical effects in MS are not fully elucidated but involve binding to CD20 which results in antibody-dependent cellular cytolysis, antibody-dependent cellular phagocytosis, apoptosis, and/or complement-mediated lysis of B cells Avivi et al. (2013) Blood Rev. 27: 217-23.

A randomized, parallel, placebo-controlled Phase II study (NCT00676715; WA21493) in patients with relapsing-remitting MS (RRMS) demonstrated that ocrelizumab is highly efficacious and well tolerated, with pronounced effects on magnetic resonance imaging and relapse-related outcomes Kappos et al. (2011) Lancet. 378: 1779-87. Two identical, pivotal Phase III studies in patients with RMS (NCT01247324; WA21092 and NCT01412333; WA21093), demonstrated the superiority of ocrelizumab over interferon beta-1a in reducing three major markers of disease activity: relapses (primary endpoint), disability progression, and brain lesion activity over the two-year controlled treatment period (Hauser et al. (2017) N Engl J Med. 376: 221-34). In a Phase III study in patients with PPMS (NCT01194570; WA25046), ocrelizumab significantly reduced the risk of confirmed disability progression sustained for at least 12 weeks (primary endpoint) and 24 weeks (key secondary endpoint) compared with placebo. Ocrelizumab treatment was also superior to placebo on other key measures of disease progression in PPMS patients including the time required to walk 25 feet, the volume of chronic brain lesions and brain volume loss (Montalban et al. (2017) N Engl J Med. 376: 209-20). Ocrelizumab is indicated for the treatment of RMS and PPMS, based on the outcomes of these pivotal studies.

This example describes a population pharmacokinetic (PK) model developed using all available patient PK data from the aforementioned Phase II trial and the two Phase III studies in RMS. The aim of this analysis was to characterize the PK of ocrelizumab, to identify covariates influencing drug exposure, and to compute individual patient exposure metrics to allow for the subsequent exploration of exposure relationships.

### Methods

### Acquisition of Data

The population PK model was developed based on data from the Phase II trial in patients with RRMS and the two Phase III studies in patients with RMS (Table A). Data from the Phase III study in PPMS (Table A) became available after model finalization and was used for external model evaluation.

TABLE A
Ocrelizumab studies included in the PK-PD analyses

| | | | | |
|---|---|---|---|---|
| | | | No. of | |
| Study no. | Study design | Population | patients | Dose, route, regimen |

Pivotal Phase III studies in RMS

| | | | | |
|---|---|---|---|---|
| WA21092 & | R, DB, DD, PG for 96 weeks (dosed every | MS according to McDonald criteria 2010 (RRMS | WA21092: | 2 arms: |
| WA21093 | 24 weeks) followed by safety follow-up or | or SPMS with relapses) | 821 | A (IV): OCR 600 mg^{a} |
| | OLE | Prior to screening: ≥2 relapses in 2 years or one | A: 410 | every 24 weeks |
| | Randomized 1:1 | relapse in the year before screening | B: 411 | B (SC): IFN 44 μg |
| | | | WA21093: | 3 times/week |
| | | | 835 | |
| | | | A: 417 | |
| | | | B: 418 | |
| WA21092 & | OLE period of WA21092 and WA21093 | From WA21092 and WA21093 | WA21092: | All patients: OCR 600 mg |
| WA21093 | (dosed every 24 weeks) | (see row above ) | 678 | every 24 weeks |
| | | | A: 352 | |
| | | | B: 326 | |
| | | | WA21093: | |
| | | | 647 | |
| | | | A: 350 | |
| | | | B: 297 | |

Pivotal Phase III study in PPMS

| | | | | |
|---|---|---|---|---|
| WA25046 | R, DB, PG for a minimum of 120 weeks | MS according to McDonald criteria 2005 (PPMS) | A: 488 | 2 arms: |
| | (dosed every 24 weeks) followed by safety | EDSS at screening 3.0 to 6.5 points | B: 244 | A (IV): OCR 2 × 300 mg |
| | follow-up or OLE Randomized 2:1 | | | (separated by 2 weeks) |
| | (OCR:placebo) | | | every 24 weeks |
| | | | | B (IV): matching placebo |

Supporting/dose finding Phase II study

| | | | | |
|---|---|---|---|---|
| WA21493 | R, PB, PC, PG, IFN, DF for 24 weeks | RRMS according to McDonald criteria 2005 | 220 | 4 arms: |
| | followed by 72 weeks OCR (dosed every | Prior to screening: ≥2 relapses in 3 years, with | A: 55 | A (IV): OCR 2000 mg (1 |
| | 24 weeks); variable treatment-free period | one relapse in the year before screening | B: 55 | dose); OCR 1000 mg (3 |
| | Randomized 1:1:1:1 | | C: 54 | doses)^{b} |
| | | | D: 54 | B (IV): OCR 600 mg |
| | | | | (4 doses)^{c} |
| | | | | C (IV): Placebo (1 dose); |
| | | | | OCR 600 mg (3 doses)^{d} |
| | | | | D (IM): IFN 30 μg; |
| | | | | OCR 600 mg (3 doses)^{e} |
| WA21493 | OLE period of WA21493 | From WA21493 (see row above) | 103 | All patients: OCR 600 mg |
| | (dosed every 24 weeks) | | A: 19 | |
| | | | B: 31 | |
| | | | C: 29 | |
| | | | D: 24 | |

^{a}Dose 1: 2× ocrelizumab 300 mg IV infusions separated by 2 weeks, subsequently 1× ocrelizumab 600 mg IV infusion every 24 weeks
^{b}Dose 1: 2× ocrelizumab 1000 mg IV infusions separated by 2 weeks; Dose 2: 1× ocrelizumab 1000 mg IV infusion and 1× placebo IV infusion separated by 2 weeks; Doses 3 and 4: 1× ocrelizumab 1000 mg IV infusion until preferred dose of 600 mg chosen following primary analysis after which point all patients were dosed with 1× ocrelizumab 600 mg IV infusion
^{c}Dose 1: 2× ocrelizumab 300 mg IV infusions separated by 2 weeks; Dose 2: 1× ocrelizumab 600 mg IV infusion and 1× placebo IV infusion separated by 2 weeks; Doses 3 and 4: 1× ocrelizumab 600 mg IV infusion
^{d}Dose 1: 2× placebo IV infusions separated by 2 weeks; Dose 2: 2× ocrelizumab 300 mg IV infusions separated by 2 weeks; Doses 3 and 4: 1× ocrelizumab 600 mg IV infusion
^{e}Dose period 1: 30 μg IFN every week; Dose 2: 2× ocrelizumab 300 mg IV infusions separated by 2 weeks; Doses 3 and 4: 1× ocrelizumab 600 mg IV infusion
DB, double-blind; DD, double-dummy; DF, dose-finding; EDSS, Expanded Disability Status Scale; IFN, interferon; IM, intramuscular; IV, intravenous; MS, multiple sclerosis; OCR, ocrelizumab; OLE, open-label extension; PB, partially blind; PC, placebo-controlled; PD, pharmacodynamics; PG, parallel-group; PK, pharmacokinetics; PPMS, primary progressive multiple sclerosis; R, randomized; RMS, relapsing multiple sclerosis; RRMS, relapsing-remitting multiple sclerosis; SC, subcutaneous; SPMS, secondary progressive multiple sclerosis; WA21092, OPERA I; WA21093, OPERA II; WA25046, ORATORIO

In the Phase II study in patients with RRMS, ocrelizumab was administered by intravenous (IV) infusion against placebo and an active control (intramuscular interferon beta-1a). Patients in the 600 mg ocrelizumab arm received 300 mg ocrelizumab IV on days 1 and 15 (total dose 600 mg) followed by single 600 mg infusions every 24 weeks. Patients in the 1000 mg ocrelizumab arm received 1000 mg ocrelizumab IV on days 1 and 15 (total dose 2000 mg) followed by 1000 mg ocrelizumab after 24 and 48 weeks, and then 600 mg every 24 weeks. Methylprednisolone (100 mg IV infusion) was given in all studies prior to each ocrelizumab infusion to reduce the risk of infusion-related reactions. Blood samples for ocrelizumab PK assessment in serum were collected 5-30 minutes prior to the methylprednisolone infusion on days 1, 15 and 169; 30 (±10) minutes after completion of the ocrelizumab infusion on days 1 and 15; on days 29, 57, 85, 113 and 141; and also at the withdrawal visit in case of early withdrawal. During the open-label extension (OLE) period, PK samples were collected prior to each infusion.

In the two Phase III studies in patients with RMS, the patients were randomized to receive either 44 μg interferon beta-1a by subcutaneous injection or 600 mg ocrelizumab IV (2×300 mg on days 1 and 15; 600 mg infusions thereafter at weeks 24, 48, and 72) followed by the OLE period with 600 mg ocrelizumab IV every 24 weeks. Blood samples for ocrelizumab PK assessment were taken pre-dose prior to the methylprednisolone infusion at weeks 1, 24, 48, and 72; 30 (±10) minutes after completion of the infusion at week 72; on days 84 and 96; and also at the withdrawal visit in case of early withdrawal. Blood samples for measurement of B cells were collected pre-dose, at week 2, week 12, and every 6 months just before the start of the next ocrelizumab infusion.

In the Phase III study in patients with PPMS, patients were randomized 2:1 to receive ocrelizumab 600 mg IV (300 mg on days 1 and 15) or placebo every 24 weeks. Patients continued to receive 600 mg doses of ocrelizumab (as 2×300 mg infusions 14 days apart) every 24 weeks until the last enrolled patient completed at least 120 weeks of study treatment and the planned total number of 253 confirmed disability progression events had been reached. Patients received a median of 7 doses of ocrelizumab during the double-blind study period. Blood samples for PK assessment were drawn pre-dose before methylprednisolone on days 1 and 15; every 6 months at weeks 24, 48, 72 and 96 just before the ocrelizumab infusion; 30 minutes after completion of the ocrelizumab infusion on days 1 and 15 and week 72; at weeks 12, 84 and 120; and on the withdrawal visit in case of early withdrawal. After week 120, samples were drawn pre-infusion before the next ocrelizumab dose. Blood samples for measurement of B cells were collected pre-dose, at week 2, week 12, and every 6 months prior to the next ocrelizumab infusion.

### Measurement of Ocrelizumab Serum Concentration

Ocrelizumab concentration in serum samples was measured with a validated enzyme linked immunosorbent assay (ELISA) with a lower limit of quantitation (LLOQ) of 250 ng/mL.

### Measurement of B Cells in Blood

B-cell count in blood was used as the pharmacodynamic (PD) marker. Because ocrelizumab binds to CD20, its presence in blood interferes with a CD20 B-cell count through interaction with the CD20 surface antigen. Therefore, CD19 was used as another B-cell surface marker that largely mirrors CD20 expression during B-cell development. The percentages and absolute counts of B, T and Natural Killer (NK) cells were determined using the BD Multitest™ 6-color TBNK reagents and BD Trucount™ tubes (Becton Dickinson, CA, USA). These allow cell staining with fluorochrome-labelled antibodies which identify T cells (CD3, CD4 and CD8), B cells (CD19), and NK cells (CD16 and CD56). Cells were then assessed by flow cytometry using a FACS Canto II cytometer (Becton Dickinson, CA, USA). Although no formal lower limit of quantitation is defined for this assay, Roche-internal data and literature (Bar-Or et al. (2018) Neurology. 90: e1805-14) suggested accuracy for B-cell counts at ≥5 cells/μL and therefore this cut-off was used for the presented analysis.

### Population PK Model

The population PK analysis was conducted via nonlinear, mixed-effects modelling using NONMEM software version 7.3.0 (ICON Development Solutions, MD, USA). The first-order conditional estimation method was used with the INTERACTION option (FOCEI). Computer resources included personal computers with Intel® processors, Windows 7 Professional operating system and Intel® Visual Fortran Professional Compiler (Version 11.0). All pre- and post-processing was performed using R version 3.1.3 for Windows (R project, /www(dot)r-project(dot)org/).

Data from the Phase II Study in RRMS Patients and the Two Phase III Studies in RMS Patients were Used for Model Development.

Previous studies have shown that mAbs targeting B cells, such as rituximab and obinutuzumab, exhibit time-dependent clearance, possibly reflecting the decreasing number of target B cells over time with treatment (Li et al. (2012) J Guth Pharmacol. 52: 1918-26; Gibiansky et al. (2014) CPT Pharmacometrics Syst Pharmacol. 3: e144). Similarly, a two-compartment model with time-dependent clearance accurately described the ocrelizumab PK. In addition, three-compartment models (a mammillary model as well as a catenary model, where the third compartment was exchanging drug with the peripheral compartment) were also tested in the current analysis in an attempt to avoid the use of time-dependent clearance. During model development, all inter-individual error terms were described by log-normal distributions, while the combined additive and proportional terms, as well as the exponential model (implemented as an additive error model in the log-transformed concentration scale), were tested for the residual error model.

Model refinement was driven by data and was based on goodness-of-fit (GOF) indicators, including various diagnostic and simulation-based predictive checks (visual predictive check [VPC] and normalized prediction distribution errors [NPDE]) plots. All parameter estimates were reported with a measure of estimation uncertainty (asymptotic standard error and 95% confidence interval [CI]). Potential covariate-parameter relationships were identified based on scientific interest, biological plausibility, exploratory analysis and exploratory graphics. The covariates investigated included body weight, age, sex, race and ethnicity, and baseline B-cell count. They were simultaneously included in the “full” model using a multiplicative expression for covariates (using normalized power models for continuous covariates). Inferences regarding covariate effects and their clinical relevance were based on the resulting parameter estimates and measures of estimation precision. Small effects (<10%) that were precisely estimated (CI within 15%) were excluded to arrive at a parsimonious model. For the data derived from the study in patients with PPMS, model diagnostics (using the same goodness-of-fit and simulation-based predictive check plots) and post-hoc estimation of the individual parameters was performed without a change in the model.

Individual concentration—time courses were simulated for all patients using individual PK parameters estimated from the model and nominal dosing. Predicted individual exposure measures (peak concentration [Cₘₐₓ], trough concentration, cumulative area under the concentration—time curve [AUC] and AUC over the dosing interval [AUC_{τ}]) were computed and summarized for each 24-week period, overall and stratified by covariates. Cₘₑₐₙ was calculated as the ratio of cumulative AUC up to the time of the last dose plus 24 weeks and duration of time from baseline until the last dose plus 24 weeks. For patients who received all planned doses, this corresponded to the Cₘₑₐₙ over the entire treatment period of 96 weeks in the RMS study. In the PPMS study, the total treatment duration varied due to the event-driven design of the study.

### Analysis of the Exposure-PD Response Relationship

Graphical analysis was performed to assess the relationship between measured blood B-cell counts, used as the PD marker of drug action, and Cₘₑₐₙ ocrelizumab as the exposure metric for all patients with RMS and PPMS. Patients were divided into four categories according to the Cₘₑₐₙ quartiles. The proportion of patients with a B-cell count of ≤5 cells/μL in each category was plotted over time and compared.

### Results

### Population PK Analysis

The PK data set consisted of 4901 quantifiable serum samples from 941 patients who received ocrelizumab (Phase II study in RRMS patients: 1182 samples from 159 patients; Phase III study in RMS patients: 1866 samples from 393 patients; parallel Phase III study in RMS patients: 1853 samples from 389 patients). The PPMS data consisted of 4340 serum samples from 482 patients enrolled in the Phase III study in PPMS patients. In addition, 739 (13%) and 424 (9%) samples in RMS and PPMS data respectively were below LLOQ (BQL), which was expected, as trough samples were taken approx. 24 weeks after the ocrelizumab infusion. These samples were not included in model development. Attempts to include BQL observations at the final stage and re-run the final model were not successful (Beal (2002) J Pharmacokinet Pharmacodyn. 29: 309).

Mean (SD) body weight for RMS was 74.8 kg (17.9) and 72.4 kg (17.2) in the PPMS study. Mean (SD) age was 37.3 years (9.17) for patients with RMS and 44.6 years (7.85) for patients with PPMS. Mean (SD) B-cell count at baseline was 0.245×10⁹/L (0.136) for patients with RMS and 0.232×10⁹/L (0.148) for patients with PPMS.

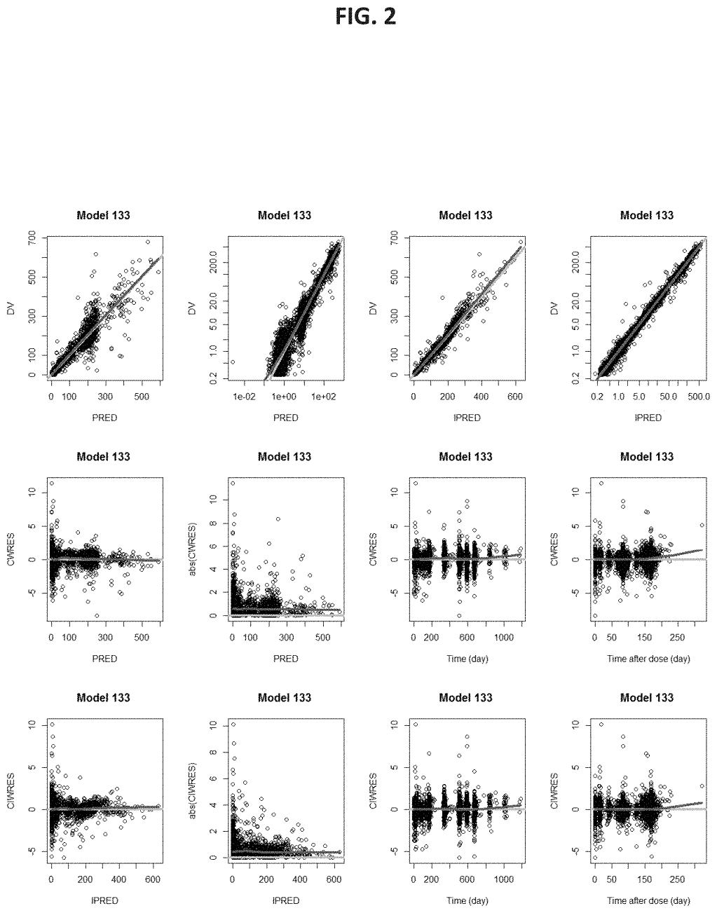

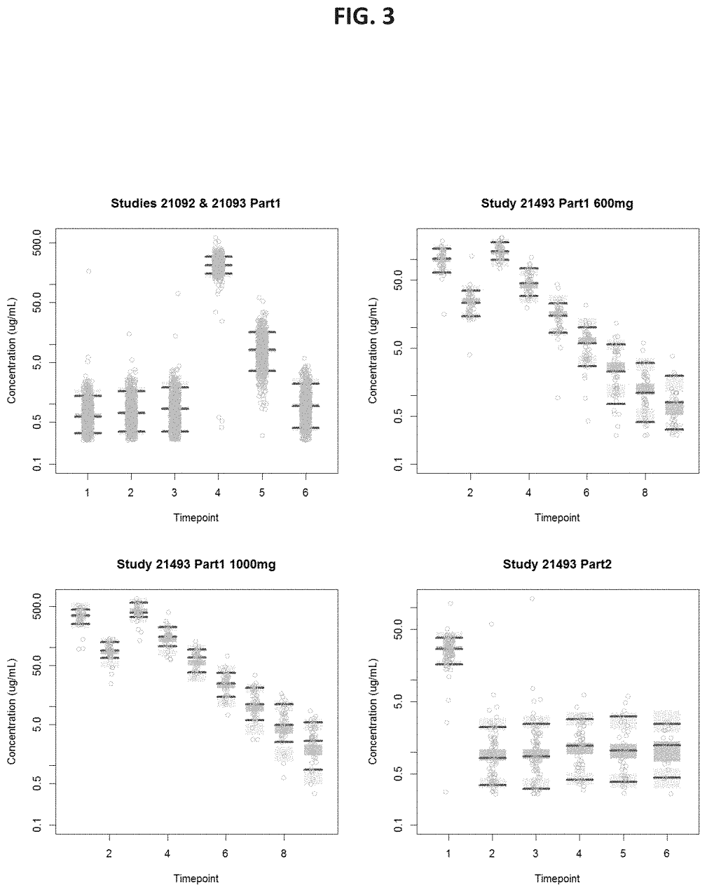

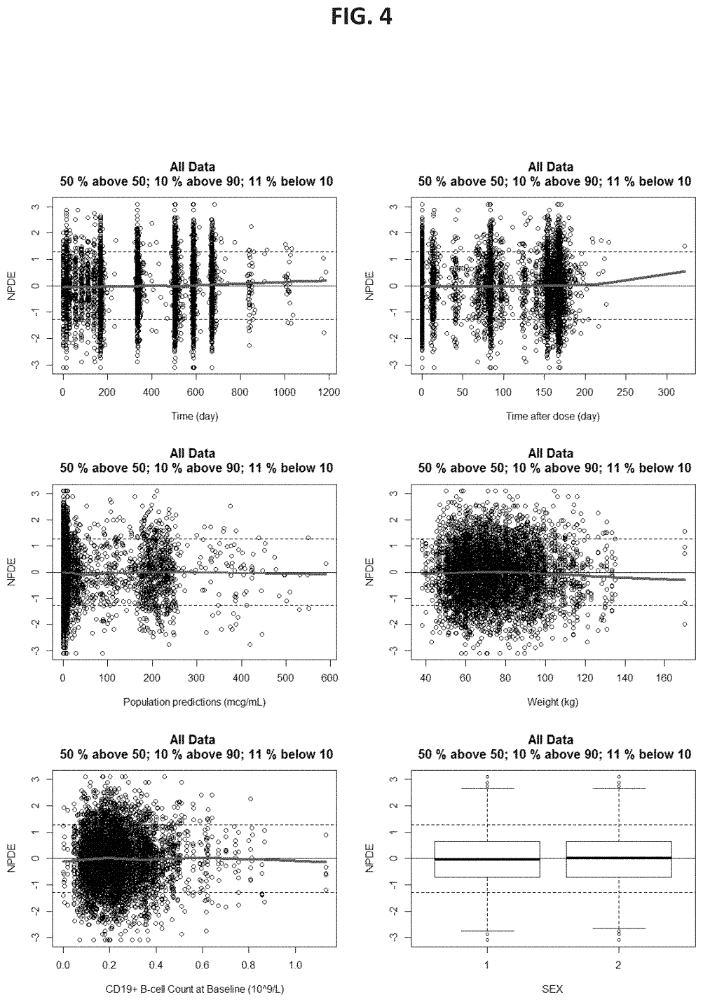

The summary of model development is presented in Table B below. The concentration—time course of ocrelizumab in patients with RMS was accurately described (see GOF, stratified VPC, and NPDE plots in FIGS. 2-4) by a two-compartment model with time-dependent clearance. Total clearance was estimated as the sum of constant clearance and time-dependent clearance, which declined exponentially with time on treatment. Estimated time-independent PK parameters were typical for an immunoglobulin G1 (IgG1) mAb (Table C).

TABLE B
Summary of NONMEM Runs for RMS Model Development

| | | | | |
|---|---|---|---|---|
| Run | Description | OFV | ΔNpar | Comment |

Base Model Development

| | | | | |
|---|---|---|---|---|
| 101 | Two-compartment linear model, etas to all | 11488.52 | — | Additive |
| | parameters, combined (additive + proportional) | | | residual error |
| | error model | | | negligible |
| 102 | As 101, but exponential residual error (additive in | −3040.53 | −1 | Accepted |
| | log transformed variables) | | | |
| 112 | As 102, but 3-comp model | −4425.20 | +2 | |
| 111 | As 102, but catenary 3-comp model | −4511.37 | +2 | |
| 103 | As 102 + time-dependent clearance | −5126.21 | +4 | Accepted |
| | (CLt = CL_{T0}*exp(−kdes*t)), eta(CL_{T0}), and separate | | | |
| | CL_{T02} for study 21493 Part2 | | | |
| 104 | As 103 + WT(CL_{inf}; V₁; CL_{T0}) + WT(Q; V₂-fixed) | −5512.31 | +3 | Accepted |
| 105 | As 104 but eta(V₂) = 0 | −5512.05 | −1 | Accepted |
| 106 | As 105 + correlation of CL_{inf} and V₁ | −5587.68 | +1 | Accepted |
| 107 | As 106 + residual error for TAD < 1 | −5595.39 | +1 | Final base |
| | | | | model |
| 108 | As 107 but error for TAD < 1 fixed to 15% | −5584.73 | −1 | Reject |
| 109 | As 106 but additive + proportional error model, | 8684.93 | +1 | Reject |
| | non-transformed variables | | | |

Covariate Model Development

| | | | | |
|---|---|---|---|---|
| 130 | As 107 + CL_{inf} and V₁(SEX; Ethn; Race) + | −5659.05 | +11 | Full model |
| | CL_{inf}(BCD19) + WT(V₂; Q) + CL_{T0}(SEX; BCD19) | | | |
| 131 | As 130, but no CL_{inf}(SEX; Ethn; Race) and | −5655.03 | −4 | Accepted |
| | CL_{T0}(SEX) | | | |
| 132 | As 131, but no V₁ (Ethn; Race); fixed Q(WT) | −5648.59 | −3 | Accepted |
| 133 | As 132, but no CL_{T0}(BCD19) | −5649.01 | −1 | Final covariate |
| | | | | model |
| 134 | As 133, but no CL_{inf}(BCD19) | −5634.50 | −1 | Rejected |
| 135 | As 133, but no V₁(SEX) | −5614.89 | −1 | Rejected |

OFV = NONMEM objective function value;
ΔNpar = Additional number of estimated parameters compared with a reference model

TABLE C
Parameter estimates of the population PK model in patients with RMS

| Parameter | | Estimate | RSE | 95% CI | | |
|---|---|---|---|---|---|---|
| CL_{inf} (L/day) | θ1 | 0.17 | 1.26 | 0.166-0.174 | | |
| V₁ (L) | θ2 | 2.78 | 1.35 | 2.71-2.85 | | |
| V₂ (L) | θ3 | 2.68 | 2.76 | 2.53-2.82 | | |
| Q (L/day) | θ4 | 0.294 | 7.46 | 0.251-0.337 | | |
| κ_{des} (year⁻¹) | θ5 | 1.11 | 5.95 | 0.979-1.24 | | |
| CL_{T0} (L/day) | θ6 | 0.0489 | 2.62 | 0.0464-0.0514 | | |
| CL_{T02} (L/day) | θ7 | 0.0199 | 8.16 | 0.0167-0.0231 | | |
| CL_{inf,WT}^{a} | θ8 | 0.684 | 5.19 | 0.615-0.754 | | |
| V_{1,WT}^{a} | θ9 | 0.397 | 8.4 | 0.331-0.462 | | |
| V_{2,WT}^{a} | θ10 | 0.853 | 6.46 | 0.745-0.961 | | |
| Q_{,WT}^{a} | θ11 | 0.75 | NA | NA | | |
| | | Fix | | | | |
| CL_{T0,WT}^{a} | θ12 | 0.981 | 7.82 | 0.831-1.13 | | |
| V_{1,Male}^{b} | θ13 | 1.12 | 2.08 | 1.07-1.16 | | |
| VL_{inf,BCD19}^{c} | θ14 | 0.0403 | 13.6 | 0.0295-0.051 | | |
| | | | | | Variability | Shrinkage |
| ω²_{CLinf} | Ω(1, 1) | 0.0535 | 5.07 | 0.0482-0.0588 | CV = 23.1% | 7.1% |
| ω_{CLinf}ω_{V1} | Ω(1, 2) | 0.026 | 11.3 | 0.0202-0.0318 | R = 0.528 | NA |
| ω²_{V1} | Ω(2, 2) | 0.0453 | 8.23 | 0.038-0.0526 | CV = 21.3% | 31.30% |
| ω²_{Q} | Ω(3, 3) | 0.239 | 8.91 | 0.197-0.281 | CV = 48.9% | 53.30% |
| ω²_{CLT0} | Ω(4, 4) | 0.125 | 12.3 | 0.095-0.156 | CV = 35.4% | 47.20% |
| σ²_{TAD≤1} | Σ(1, 1) | 0.0346 | 9.01 | 0.0285-0.0407 | CV = 18.6% | 28.7% |
| σ²_{TAD>1} | Σ(2, 2) | 0.0487 | 1.31 | 0.0474-0.0499 | CV = 22.1% | 17.9% |

^{a}Power coefficient of the power function with the reference value of 75 kg
^{b}Multiplicative factor for the respective subpopulation compared with the rest of the patients
^{c}Power coefficient of the power function with the reference value of 0.225 × 10⁹/L
% RSE, relative standard error; σ², sigma², residual variance; ω², omega², inter-individual variance; CI, confidence interval;
CL_{inf}, constant clearance;
CL_{T0}, initial time-dependent clearance (at time 0);
CL_{T02}, initial time-dependent clearance at the start of OLE for Phase II study following partial B-cell recovery (time was reset to zero);
CV, coefficient of variation computed as 100% multiplied by the square root of the variance;
NA, not applicable;
OLE, open-label extension;
PK, pharmacokinetic;
Q, inter-compartmental clearance;
R, correlation coefficient;
RMS, relapsing multiple sclerosis;
RSE, 100 · SE/PE, where PE is parameter estimate;
SE, standard error;
TAD, time after dose (days);
V₁, central volume;
V₂, peripheral volume

For a reference patient (female, 75 kg, baseline B-cell count 0.225×10⁹/L), ocrelizumab time-independent clearance and central volume were estimated at 0.17 L/day (95% CI: 0.166-0.174) and 2.78 L (95% CI: 2.71-2.85), respectively. Initial time-dependent clearance was estimated at 0.0489 L/day (95% CI: 0.0464-0.0514), comprising 20% of the total initial clearance, and declined with a half-life of 33 weeks. The estimated terminal half-life of ocrelizumab was 26 days.

Body weight was identified as the main covariate (Table D). Cₘₐₓ values were estimated to be 19% higher for patients weighing <60 kg and 13% lower for patients weighing >90 kg when compared with the 60-90 kg group. AUC_{τ} was estimated to be 26% higher for patients weighing <60 kg and 21% lower for patients weighing >90 kg when compared with the 60-90 kg group. Higher clearance was also identified in patients with a higher B-cell count at baseline (<7% increase at the 97.5th percentile), and central volume was higher (<12% increase) in males vs. females.

TABLE D
Covariate effects for the population
PK model in patients with RMS

| Par- | | Reference | Covariate | Covariate effect value | |
|---|---|---|---|---|---|
| ameter | Covariate | value | value^{a} | [95% CI] (%) | |
| CL_{inf} | Body weight | 75 | 48.5 | −25.8 | [−23.5; −28] |
| | (kg) | | 116 | 34.8 | [30.7; 38.9] |
| | B-cell count at | 0.225 | 0.0715 | 2.7 | [−2; −3.5] |
| | baseline | | 0.598 | 6.7 | [4.9; 8.5] |
| | (10⁹/L) | | | | |
| V₁ | Body weight | 75 | 48.5 | −15.9 | [−13.4; −18.2] |
| | (kg) | | 116 | 18.9 | [15.5; 22.3] |
| | Sex | Female | Male | 11.7 | [7.2; 16.3] |
| CL_{T0} | Body weight | 75 | 48.5 | −34.8 | [−30.4; −38.9] |
| | (kg) ) | | 116 | 53.4 | [43.7; 63.8] |
| V₂ | Body weight | 75 | 48.5 | −31.1 | [−27.7; −34.2] |
| | (kg) | | 116 | 45.1 | [38.4; 52.1] |
| Q | Body weight | 75 | 48.5 | −27.9 | [−27.9; −27.9] |
| | (kg) | | 116 | 38.7 | [38.7; 38.7] |

^{a}Values of the continuous covariates represent 2.5th and 97.5th percentiles of the values in the analysis data set.
CI, confidence interval; CL_{inf}, constant clearance; CL_{T0}, time-dependent clearance; PK, pharmacokinetic; Q, inter-compartmental clearance; RMS, relapsing multiple sclerosis; V₁, central volume; V₂, peripheral volume

All model parameters were estimated precisely (relative standard error <14%) and inter-individual variability was low (coefficient of variation [CV]≤35%, except for inter-compartmental clearance [Q], for which CV was 50%).

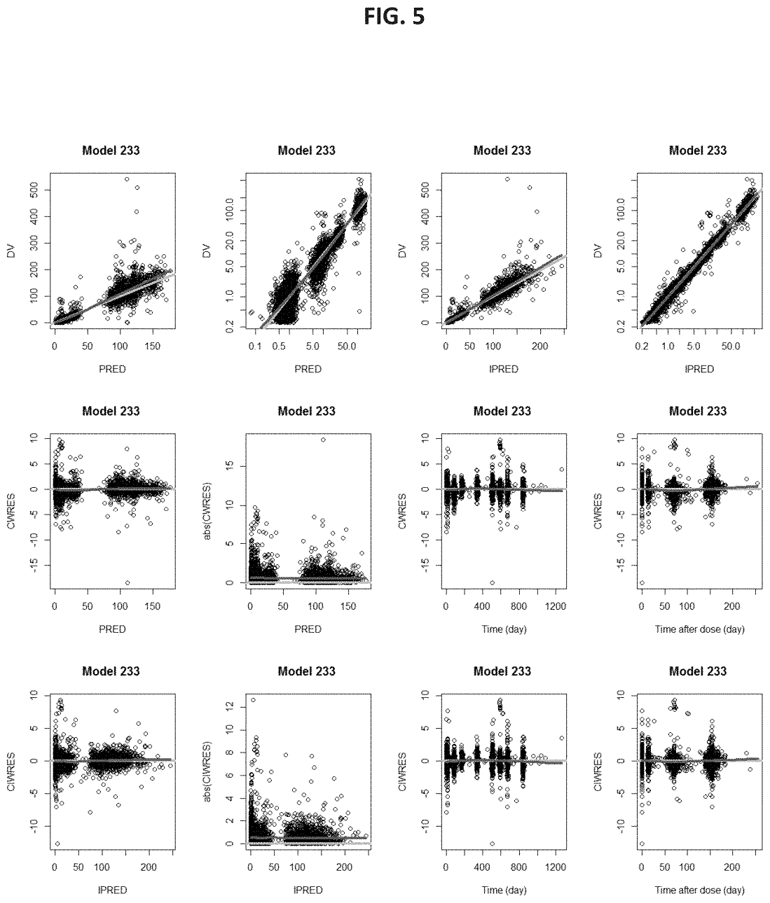

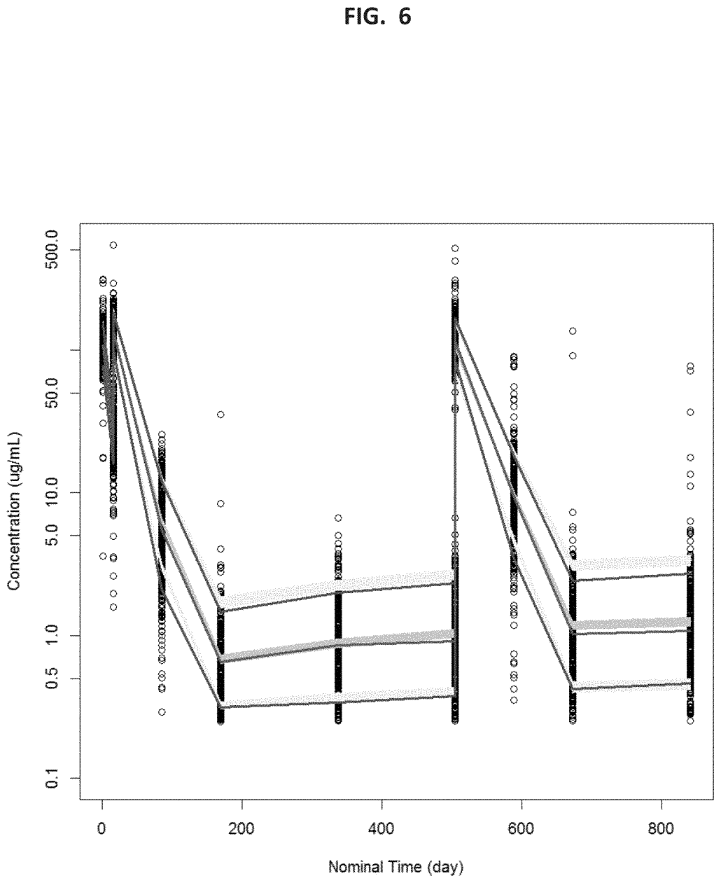

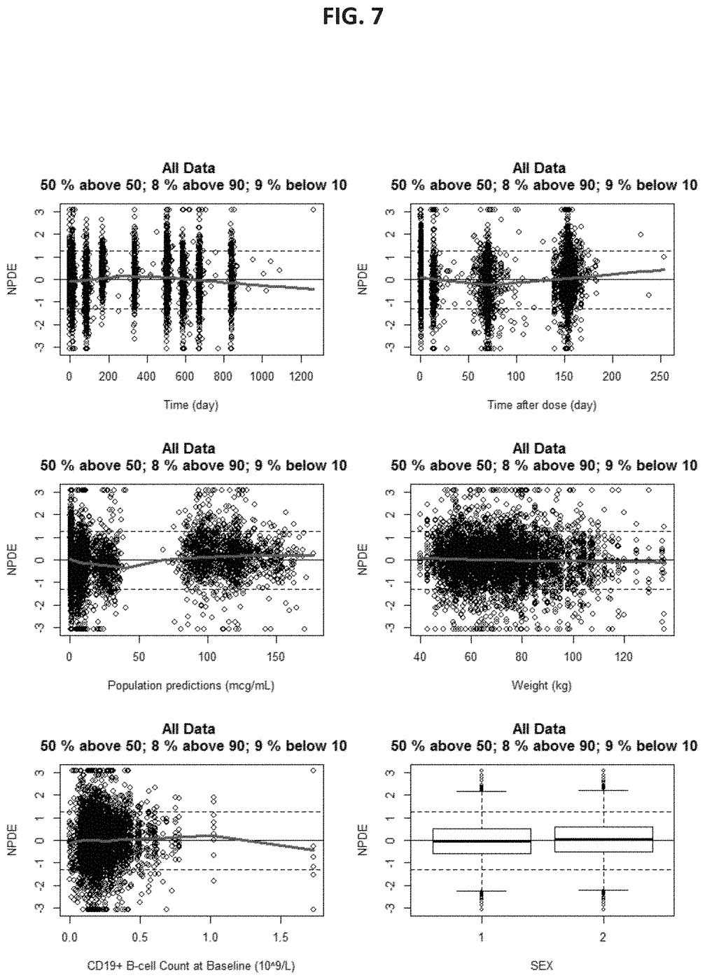

The model developed based on the RMS data also accurately described ocrelizumab concentrations as well as effects of covariates in patients with PPMS (FIGS. 5-7), thus, re-estimation of PK parameters and covariate effects was not performed for the PPMS data.

Ocrelizumab PK was independent of age and renal and hepatic function within the given data set, based on comparison of estimated PK parameters for these patients.

Only 1% of the population tested positive for treatment-emergent antidrug antibodies (ADA) during the controlled treatment period (three patients in the RMS Phase III studies, nine patients in the PPMS trial). Upon visual inspection, their PK data was comparable to ADA-negative patients and therefore remained in the data set; no formal covariate testing was performed due to the small numbers.

Ethnicity and race had no impact on PK; the vast majority of patients was, however, categorized as White.

In addition to describing the PK of the 600 mg dose, the obtained PK parameters were applied to explore alternative dosing regimens via PK simulations. Table E1 shows that a dosing regimen equivalent to 600 mg administered as mg-per-kg-body-weight (i.e. 8 mg/kg) does not reduce PK variability in a relevant manner, and therefore does not have any advantage over the currently approved 600 mg regimen.

TABLE E1
Simulated exposure (Cₘₑₐₙ) distribution
for alternative dosing regimens

| Alternative Dosing Regimen | | Mean | Median | 5^{th} tile | 95^{th} tile |
|---|---|---|---|---|---|
| 600 | mg | 19.3 | 18.9 | 11.8 | 28.1 |
| 1200 | mg | 38.6 | 37.9 | 23.7 | 56.3 |
| 1800 | mg | 57.9 | 56.8 | 35.5 | 84.4 |
| 8 | mg/kg | 18.2 | 18.1 | 12.6 | 24.6 |
| 16 | mg/kg | 36.5 | 36.2 | 25.2 | 49.2 |
| 24 | mg/kg | 54.7 | 54.3 | 37.8 | 73.7 |

| | | | | |
|---|---|---|---|---|
| 1200 mg (<70 kg) & | 42.9 | 42.5 | 28.2 | 59.2 |
| 1500 mg (>=70 kg) | | | | |
| 1200 mg (<75 kg) & | 41.9 | 41.4 | 27.4 | 58.3 |
| 1500 mg (>=75 kg) | | | | |
| 1200 mg (<75 kg) & | 45.2 | 44.4 | 29.5 | 63.3 |
| 1800 mg (>=75 kg) | | | | |

### Analysis of the Exposure-PD Response Relationship

Treatment with ocrelizumab led to rapid depletion of CD19-positive B cells in blood (measured 14 days post-infusion, the first time point of assessment), and B-cell depletion was sustained for the duration of treatment for the majority (96%) of patients. Only up to 4% of patients showed B cell repletion (above the lower limit of normal (LLN), defined as 80 cells/4, or their respective baseline measurement, whichever was lower) between the ocrelizumab doses given every 6 months. A dosing interval of 24 weeks (6 months) had indeed been selected previously as the dosing regimen for ocrelizumab treatment, with very few patients repleting B cells between doses, as observed in previous studies with ocrelizumab in patients with rheumatoid arthritis (RA), to ensure in general continuous depletion of peripheral blood B cells throughout treatment.

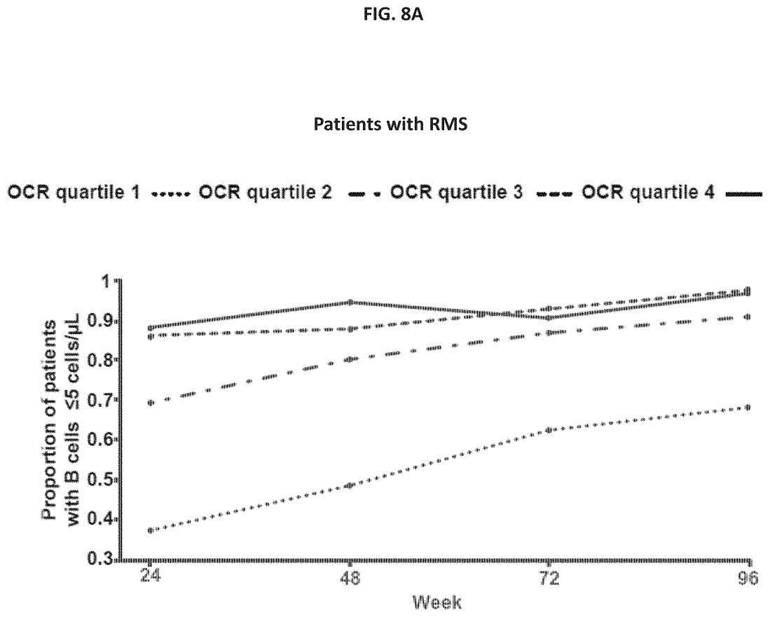

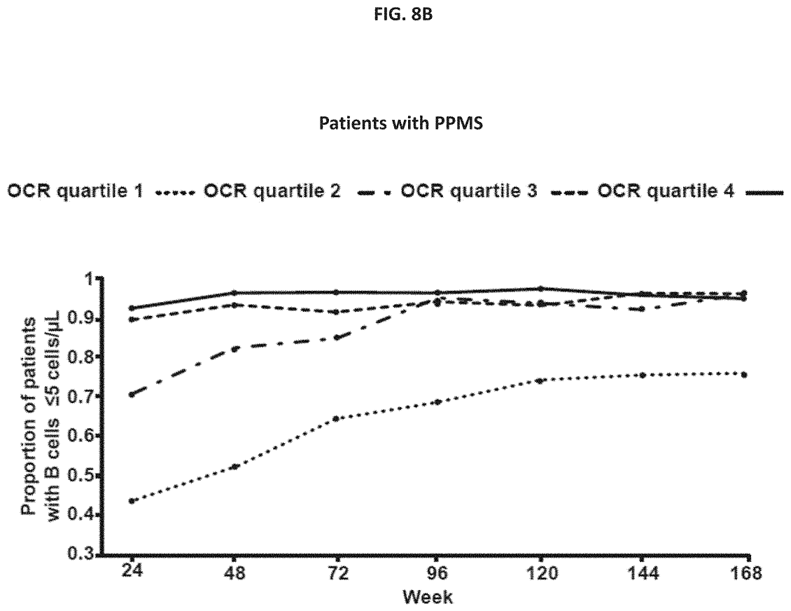

Differences in B-cell depletion were observed across exposure quartiles for the proportion of patients achieving B-cell depletion in blood of ≤5 cells/4 (level of assay accuracy for B-cell counts (Bar-Or et al. (2018) Neurology. 90: e1805-14)) at the assessed time points. The initial decrease in B cells was larger and the return of B cells before the next treatment lower in higher Cₘₑₐₙ quartiles compared with the lower quartiles. FIGS. 8A and 8B show the fraction of patients with RMS and PPMS with blood B-cell levels of ≤5 cells/μL over time by Cₘₑₐₙ quartiles. Although all patients presented with extensive B-cell depletion in blood after treatment with ocrelizumab, this analysis showed more pronounced B-cell depletion in patients with higher exposure, and improved B-cell depletion over time with continued treatment. More than 90% of all patients with RMS or PPMS in the two top exposure quartiles achieved blood B-cell levels of ≤5 cells/μL by 96 weeks, whereas in the lowest exposure quartile less than 70% of all patients were in this category at week 96.

Time to repletion could not be assessed from the pivotal studies as the majority of patients elected to continue receiving treatment with ocrelizumab in the OLE. However, repletion data from the Phase II study show that, following the final infusion of 600 mg ocrelizumab, median time to B-cell repletion was 72 weeks (range 27-175). B-cell levels returned to above the LLN (80 cells/4) or baseline measurement (whichever was lower) by approximately 120 weeks (2.5 years) after the last infusion in 90% of patients.

Given that the greatest extent of B cell depletion was observed in the highest ocrelizumab exposure quartile, simulations were conducted to explore which dose brings the most patients into the range of the highest PK quartile, but without exceeding the exposure range previously assessed in clinical trials (Table E1). A mg/kg dosing regimen did not change the range of PK exposure versus the corresponding flat dose (e.g. 8 mg/kg versus 600 mg) and has therefore no advantage. However, a dosing regimen with two different dose levels split according to a patient's body weight, above or below 70 kg or 75 kg, is interesting option to achieve such a scenario.

### DISCUSSION

The concentration—time course of ocrelizumab in patients with RMS was accurately described by a two-compartment PK model with time-dependent clearance. The model was also able to accurately predict the PK of ocrelizumab in patients with PPMS.

The presence of a time-dependent clearance component is likely due to target-mediated drug disposition (TMDD). Clearance of ocrelizumab is mediated in part by its therapeutic target, CD20-positive B cells. As treatment continues and B cells are depleted, the contribution of TMDD to the overall clearance is reduced. Following a longer interruption in treatment, as was the case between the main treatment phase and the OLE in the Phase II study, partial restoration of B cells is observed; this is accompanied by a corresponding partial restoration of the time-dependent clearance, adding further evidence to the TMDD hypothesis.

Population PK models developed to describe the PK of other anti-CD20 agents, such as obinutuzumab and rituximab, have shown that clearance of these molecules similarly consists of both time-dependent and time-independent components (Gibiansky et al. (2014) CPT Pharmacometrics Syst Pharmacol. 3: e144; Rozman et al. (2017) Br J Clin Pharmacol. 83: 1782-90; Struemper et al. (2014) J Clin Pharmacol 54: 818-27). With the data presented here, the time-dependent clearance component accounted for approximately 20% of the total initial clearance. All estimated time-independent PK parameters were typical for an IgG1 mAb (Mould et al. (2007) Curr Opin Drug Discov Devel. 10: 84-96).

In the Phase III trial in patients with PPMS, patients received the 600 mg ocrelizumab dose as two infusions of 300 mg 14 days apart throughout the study. The dosing regimen evaluated in the Phase III trials in MS had been chosen based on PK, PD, immunogenicity, safety and efficacy data obtained with ocrelizumab in prior RA studies and the Phase II study in patients with RRMS (Huffstutter et al. (2011) Int J Clin Rheumatol. 6: 689-96). In the Phase III studies in patients with RMS and in the Phase III study in patients with PPMS, overall ocrelizumab exposure (AUC) was identical with the single-infusion (600 mg) and the split-infusion (2×300 mg) regimens. The observed B-cell depletion in blood, the pattern of only <4% patients with B-cell repletion between ocrelizumab doses administered every 6 months, and the PK-PD correlation was comparable in the RMS and PPMS trials, independent of the dosing regimen used. This indicated that there appears to be no benefit to administering ocrelizumab as double infusions after the first dose. The first dose is however maintained as 2×300 mg infusions given 2 weeks apart, to potentially reduce the risk for infusion-related reactions which occur most frequently upon the first ocrelizumab administration. A harmonized dosing regimen (with the first 600 mg dose always given as 2×300 mg infusions, and subsequent doses as single 600 mg infusions) has been approved by all health authorities for all patients with RMS and PPMS. No dose adjustment was considered necessary to account for the identified covariate effects.

Treatment with ocrelizumab 600 mg led to rapid and near-complete depletion of B cells in blood, which was sustained throughout treatment for the vast majority of patients. More patients in the two highest quartiles of ocrelizumab exposure had B-cell levels ≤5 cells/4 when compared with the lowest quartile. B-cell depletion in the lower exposure groups improved over time with further subsequent ocrelizumab dose administrations. These data indicate that the 600 mg every 24 weeks ocrelizumab dosing regimen achieves generally near-complete B-cell depletion overall, but there is an exposure correlation and patients in the highest quartiles show the lowest B cell count. Several doses of ocrelizumab treatment may be required to achieve deeper depletion of B cells in blood and other body compartments over time, as only a minority of B cells are located in the blood, while the vast majority of B cells reside in tissues. There is no established specific B cell depletion target. Baseline B cell count in MS patients is within the normal range. Dose selection for ocrelizumab was done based on the clinical (efficacy & safety) outcomes of the previously conducted Phase 2 study, and are not based on a specific target B cell count. The value of </=5 cells/μL has been chosen as the cut-off for a reliable measurement of B cells in blood, i.e. below this value B depletion in blood in considered complete. PK simulations were conducted for alternative dosing regimens, in view of the presented exposure response on B cells in blood and the correlation of PK with body weight. It is however currently unknown whether a different dosing regimen could be beneficial to obtain further improved efficacy of ocrelizumab. Currently no other dosing recommendations can be given, as only the 600 mg dose was evaluated in clinical trials. Further assessment is required to better understand any potential relationship between B-cell levels in blood and efficacy parameters. In addition, while the relationship of B-cell levels in blood based on exposure is informative at the population level in a highly harmonized clinical trial setting, individual patients' B-cell measurements can be variable and thus lack sensitivity to inform treatment decisions.

In conclusion, the pharmacokinetics of ocrelizumab was described with pharmacokinetic parameters typical for an immunoglobulin G1 monoclonal antibody, with body weight as the main covariate. The pharmacokinetics and B-cell depletion in blood were comparable across the RMS and PPMS trials, with near-complete B-cell depletion overall. The greatest B cell depletion was observed for patients with the highest ocrelizumab exposure. The current dosing regimen of 600 mg ocrelizumab every 6 months has been shown to lead to significant efficacy in the Phase III studies and has been approved world-wide for treatment of RMS and PPMS patients. It is currently unknown whether other dosing regimens could further improve ocrelizumab's efficacy.

### Example 2A: Rationale and Design of Two Phase IIIb Studies of Ocrelizumab at Higher than the Approved Dose in Patients with RMS and PPMS

### Background

Ocrelizumab (OCR) is approved for the treatment of relapsing (RMS) and primary progressive multiple sclerosis (PPMS) at a dose of 600 mg IV twice-yearly and showed significant benefit on disability progression (DP). Exposure-response (ER) analyses of the pivotal OCR Phase III studies in patients with RMS or PPMS showed that those with higher exposures (based on individual mean serum concentration [Cmean] exposure quartiles) had a greater benefit on DP vs patients with lower exposure, without an increase in adverse events. While doses of OCR of 1000-2000 mg were studied in a Phase II study, doses >600 mg have not been investigated in Phase III studies in RMS or PPMS patients.

### Objective

To present the OCR higher dose selection rationale and design of two double-blind, parallel-group, randomised Phase IIIb studies (one in RMS and one in PPMS) of higher dose OCR vs 600 mg on DP without adversely affecting the established favourable benefit-risk profile.

### Methods

The higher dose of OCR in both studies is based on achieving a Cₘₑₐₙ of at least that observed in the highest exposure quartile of the Phase III ER analyses while limiting Cₘₑₐₙ below that observed with the highest OCR dose of 2000 mg in the Phase II study that had a similar safety profile, except for a slightly higher incidence of infusion-related reactions (pre-medication: methylprednisolone only; no mandatory antihistamine).

### Results

Modelling predicts that doses of 1200 mg (patients <75 kg) or 1800 mg (patients ≥75 kg) twice yearly fulfils these criteria. Based on data from the pivotal trials, the expected risk reduction vs 600 mg in 12-week composite confirmed DP (cCDP, consisting of time to progression measured by the EDSS, Timed 25-Foot Walk or 9-Hole Peg Test) would be ≥35% in RMS and ≥27% in PPMS. Patients with RMS (EDSS score 0-5.5; N=786) or PPMS (EDSS score ≥3.0-6.5; N=699) are randomised (2:1) to either the higher dose (as above) or OCR 600 mg administered every 24 weeks (first dose divided into 2 infusions separated by 14 days) for ≥120 weeks (minimum 5 doses).

The primary outcome for both trials is risk reduction on cCDP. Immunoglobulin and oligoclonal bands in the CSF are assessed in a sub-study of up to 288 patients.

### Conclusions

It is expected that higher-dose OCR provides an even higher benefit on cCDP vs the approved 600 mg dose without adversely affecting the established favourable benefit-risk profile.

### Example 2B: Further Details Regarding the Rationale and Design of Two Phase IIIb Studies of Ocrelizumab (OCR) at Higher than the Approved Dose in Patients with RMS and PPMS

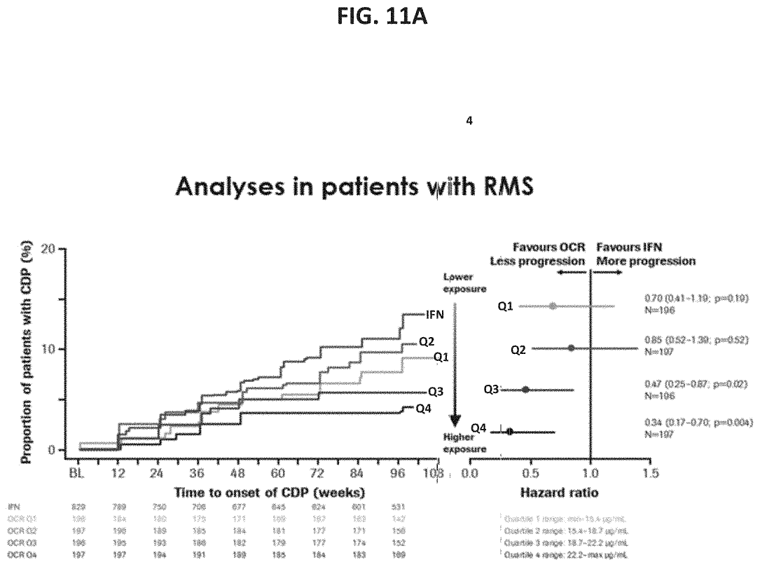

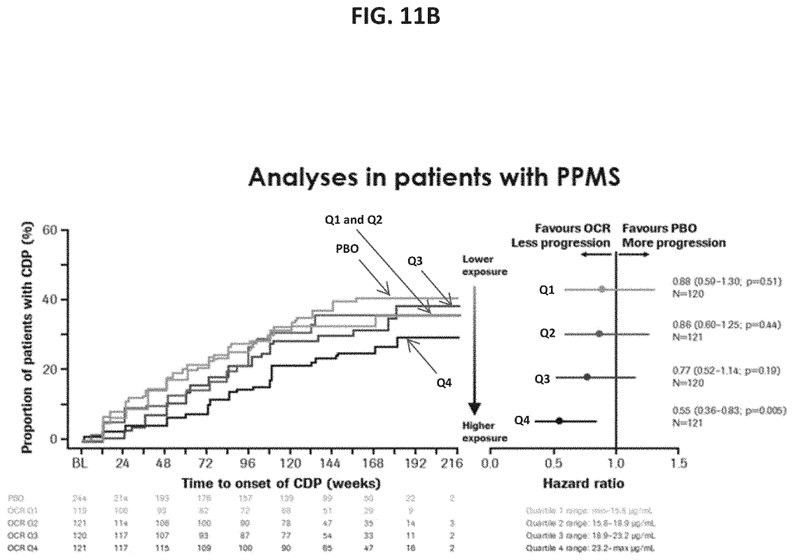

OCR was the first anti-CD20 monoclonal antibody approved at a dose of 600 mg IV twice yearly, for the treatment of RMS and PPMS; it remains the only approved treatment for PPMS (OCREVUS [ocrelizumab] Full Prescribing Information. Genentech, Inc., 2020; OCREVUS [ocrelizumab] Summary of Product Characteristics. Roche Pharma AG, 2020). OCR had significant benefit on 12 week and 24 week confirmed disability progression (12/24 w CDP), annualized relapse rate (ARR), and MRI measures in pivotal Phase III studies in patients with RMS (Hauser S L, et al. N Engl J Med 2017:376:221-234) or PPMS (Montalban X, et al. N Engl J Med 2017; 376:209-220) with sustained efficacy in the respective open-label extension periods. Post-hoc ER analyses of the pivotal Phase III studies showed that patients with OCR higher exposures had a greater benefit on 12/24W-CDP versus patients with lower exposure. See FIGS. 11A and 11B. Exposures were based on individual patient mean serum concentrations. The clinical benefit on ARR, the safety profile and the rate of IgG decline were similar across exposure quartiles. Safety measures included adverse events, serious adverse events and serious infections.

The objective of the present example is to examine how a higher dose of OCR could further decrease the risk of disability progression without compromising the established benefit-risk profile of the approved dose in patients with RMS or PPMS. This example provides a rationale for OCR higher dose selection and the design of two double-blind, parallel-group, randomized Phase Mb studies testing the efficacy and safety of a higher dose of OCR in patients with RMS or PPMS.

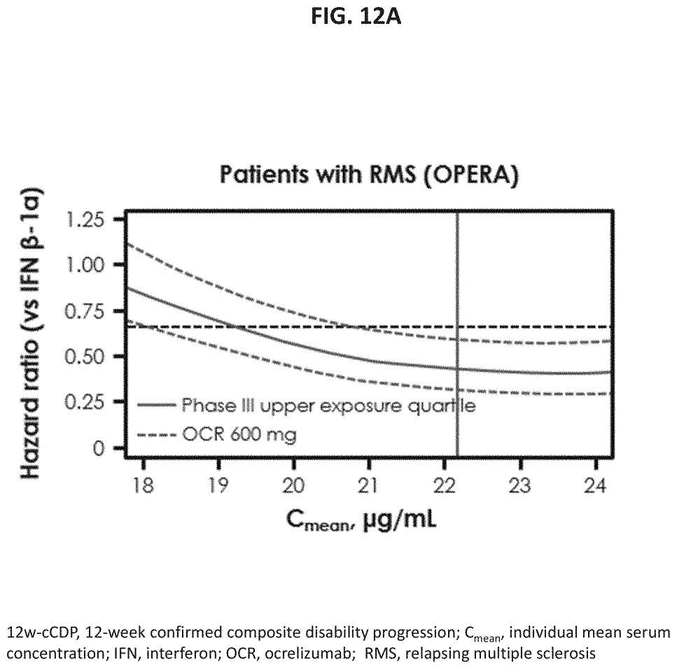

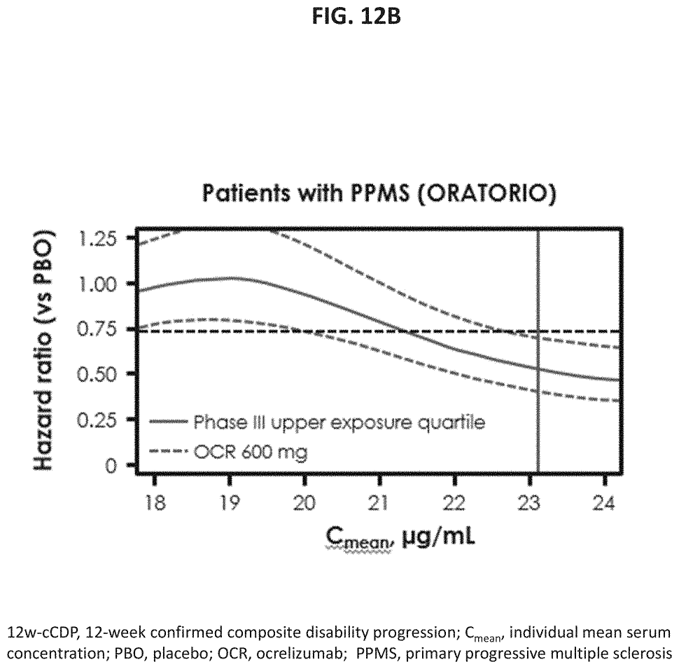

The dose-ranging rationale had two considerations. The first consideration was upper exposure limit, i.e., to maintain exposure within the known safety profile by limiting exposure to the highest Phase II dose exposure of 2,000 mg; 83 μg/mL. Phase II OCR 2,000 mg safety outcomes were comparable to the approved 600 mg dose (a higher rate of IRRs was observed, pre-medications for IRRs did not include the mandatory use of antihistamines at the time of the Phase II study). The second consideration was lower exposure limit, i.e., to target an exposure of at least the highest exposure quartile in the Phase III pivotal studies (RMS, 22.2 μg/mL or PPMS, 23.1 μg/mL) and achieve a minimal improvement in 12 week composite confirmed disability progression (12 w-cCDP) risk reduction in patients with RMS (≥56% vs. interferon beta) or PPMS (≥46% vs. placebo). The relationship between exposure and 12 w-cCDP was predicted using Phase III pivotal study data. See FIGS. 12A and 12B.

Population PK modelling of Phase III data was used to model potential higher-dose regimens and their exposure distributions. Several regimens were modelled to achieve the exposure observed within the upper quartile of the pivotal Phase III studies, gain a benefit on 12 w-cCDP and maintain exposure within the known safety window. See Table E2 below for examples of regimens explored.

TABLE E2
Summary statistics of Cₘₑₐₙ distribution and efficacy properties of explored doses

| | Patients with RMS | | | Patients with PPMS | | |
|---|---|---|---|---|---|---|
| | Patients | Patients | Estimated | Patients | Patients | |
| | achieving | exceeding | minimum | achieving | exceeding | Estimated |
| | minimum | maximum | treatment | minimum | maximum | minimum |
| | exposure | exposure | effects^{b} | exposure | exposure | treatment |
| | (Cₘₑₐₙ > 22.2 | (Cₘₑₐₙ > 83 | vs IFN | (Cₘₑₐₙ > 23.1 | (Cₘₑₐₙ > 83 | effects^{b} |
| Regimen | μg/mL) (%) | μg/mL) (%) | β-1a | μg/mL) (%) | μg/mL) (%) | vs PBO |
| 1,200 mg | 97.06 | 0.26 | 0.62 | 94.61 | 0.21 | 1.02 |
| (Independent | | | (0.48, 0.79) | | | (0.80, 1.30) |
| of body | | | | | | |
| weight) | | | | | | |
| 1,800 mg | 100 | 4.86 | Not | 99.17 | 6.43 | Not |
| (Independent | | | estimable | | | estimable |
| of body | | | | | | |
| weight) | | | | | | |
| 1,200 mg in | 99.61 | 0.26 | 0.43 | 98.76 | 0.42 | 0.54 |
| patients < 75 | | | (0.30, 0.61) | | | (0.41, 0.71) |
| kg & 1,800 | | | | | | |
| mg in | | | | | | |
| patients ≥75 | | | | | | |
| kg | | | | | | |

12w-cCDP, 12-week confirmed composite disability progression;
PBO, placebo;
Cₘₑₐₙ, individual mean serum concentration;
IFN, interferon;
PK, pharmacokinetic;
PPMS, primary progressive multiple sclerosis;
RMS, relapsing multiple sclerosis

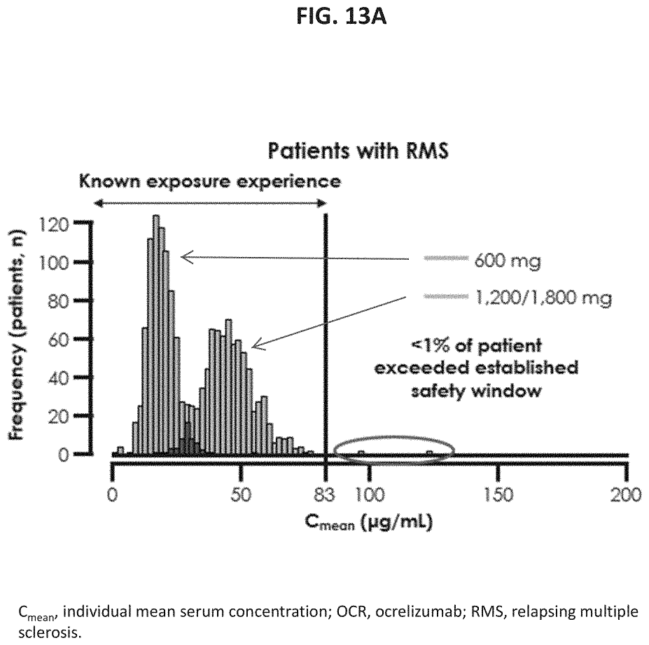

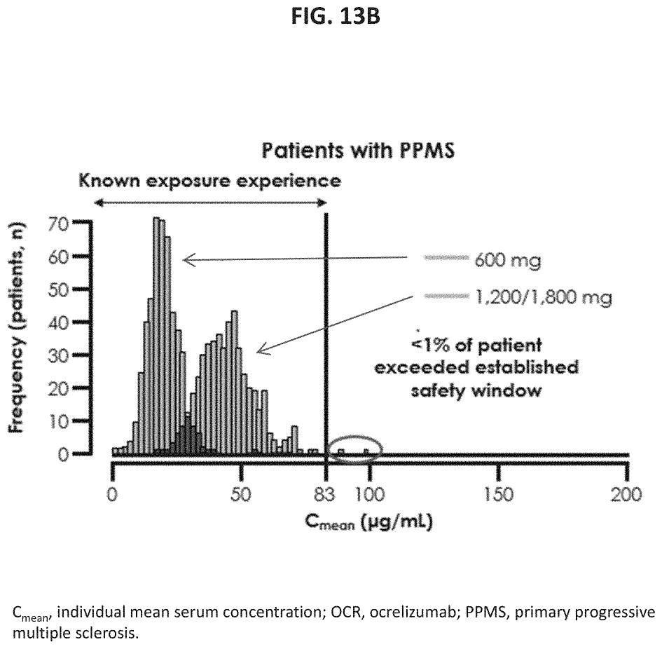

Predicted Cₘₑₐₙ distributions with the modelled Cₘₑₐₙ/12 w-cCDP relationship were used to estimate the modelled improvement in 12 w-cCDP. The data in Table E2 are hazard ratio (95% confidence interval) of having 12 w-cCDP relative to the study comparator. The dose of 1,200 mg in patients <75 kg or 1,800 mg in patients ≥75 kg was found to be optimal to achieve the desired modelled exposure with consideration of efficacy and safety outcomes. As shown in FIGS. 13A and 13B, the weight cut-off ensures that fewer than 1% of patients would have exposures exceeding the established safety window of OCR

Higher dose OCR studies in patients with RMS or PPMS are described in further detail in Examples 3 and 4.

### Conclusions

OCR was the first anti-CD20 monoclonal antibody approved at a dose of 600 mg IV twice yearly, for the treatment of RMS and PPMS; it remains the only approved treatment for PPMS. OCR had significant benefit on 12/24W-CDP, ARR, and MRI measures in pivotal Phase III studies in patients with RMS or PPMS with sustained efficacy in the respective open-label extension periods. Exposure response analyses of Phase III data suggest that a higher dose of ocrelizumab could lower the risk of disability progression without compromising the benefit-risk profile of the approved dose. Two double-blind, parallel-group, randomized Phase IIIb studies, one in RMS (Example 3) and one in PPMS (Example 4), have been designed to explore the effect of a higher dose of ocrelizumab, given every 24 weeks, on the risk of disability progression. The selected ocrelizumab higher dose is 1,200 mg for patients <75 kg or 1,800 mg for patients ≥75 kg.

### Example 3: A Phase IIIb Multicenter, Randomized, Double-Blind, Controlled Study to Evaluate the Efficacy, Safety and Pharmacokinetics of a Higher Dose of Ocrelizumab in Adults with Relapsing Multiple Sclerosis (RMS)

This example describes a Phase IIIb, randomized, double blind, controlled, parallel group, multicenter study to evaluate efficacy, safety and pharmacokinetics of a higher dose of ocrelizumab (1200 mg [patient's body weight <75 kg] or 1800 mg [patient's body weight ≥75 kg]) per IV infusion every 24 weeks (6 months) in patients with RMS, in comparison to the approved 600 mg dose of ocrelizumab.

### I. Efficacy Objectives

### (a) Primary Efficacy Objective

The primary efficacy objective is to demonstrate the superiority of a higher dose of ocrelizumab over the approved dose of ocrelizumab as assessed by risk reduction in composite confirmed disability progression (cCDP) sustained for at least 12 weeks. The comparison of interest is the difference in time to 12-week cCDP (cCDP12), as expressed by the hazard ratio. The primary comparison is made regardless of adherence to the randomized treatment or use of alternative MS treatment.

Time to onset of cCDP is defined as the first occurrence of a confirmed progression event according to at least one of the following three criteria:
CDP, defined as a sustained increase from baseline in Expanded Disability Status Scale (EDSS) score of ≥1.0 point in patients with a baseline EDSS score of 5.5 or a sustained increase ≥0.5 points in patients with a baseline EDSS score of >5.5, or score, or
A sustained increase of ≥20% from baseline in Timed 25-Foot Walk Test (T25FWT)
A sustained increase of ≥20% from baseline in time to complete the 9-Hole Peg Test (9-HPT) score.

The EDSS is a disability scale that ranges in 0.5-point steps from 0 (normal) to 10.0 (death) (Kurtzke (1983) Neurology. 33:1444-52; Kappos (2011) Neurology, University Hospital Basel, Switzerland: Neurostatus Scoring Definitions). The baseline EDSS score is calculated as the average of the EDSS scores at screening and the Day 1 visit.

The T25FWT and 9-HPT scores are calculated as described in the MS functional composite guide (National Multiple Sclerosis Society 2001, see www(dot)nationalmssociety(dot)org/For-Professionals/Researchers/Resources-for-Researchers/Clinical-Study-Measures/9-Hole-Peg-Test-(9-HPT))

The score for the timed T25FWT is the average of the two completed trials. The most recent timed T25FWT score measured prior to randomization is considered as baseline.

The score for the 9-HPT is an average of the four trials. The two trials for each hand are averaged, converted to the reciprocal of the mean time for each hand, and then two reciprocals are averaged and back-transformed to the original scale (i.e., by taking another reciprocal). The most recent 9-HPT score measured prior to randomization is considered as baseline.

Additional details regarding the administration and/or scoring of EDSS, T25FWT, and 9-HPT are described below.

### (b) Secondary Efficacy Objective

The secondary efficacy objective is to demonstrate superiority of a higher dose of ocrelizumab over the approved dose of ocrelizumab on the basis of the following endpoints:
Time to onset of 24-week cCDP (cCDP24);
Time to onset of 12-week composite disability progression (CDP) (CDP12);
Time to onset of 24-week CDP (CDP24);
Time to ≥20% increase in 12-week confirmed T25FWT;
Time to ≥20% increase in 24-week confirmed T25FWT;
Change from baseline in the Multiple Sclerosis Impact Scale (MSIS-29) physical scale (i.e., a 29 item patient-reported measure of the physical and psychological impacts of MS) at Week 120;
Percent change in total brain volume from Week 24 to Week 120;
Time to 12-week confirmed 4-point worsening in Symbol Digit Modality test (SDMT);

For all time to event endpoints, the comparison of interest is the difference in time to event between treatment arms, as expressed by the hazard ratio. For all other endpoints, the comparison of interest is the difference in variable means between treatment arms. All comparisons, except for the MRI endpoint (i.e. the change in brain volume), are made regardless of adherence to the randomized treatment or use of alternative MS treatment. For the MRI endpoint, the comparison is made as if no treatment discontinuation or switch to alternative MS treatment occurs.

Details regarding the administration and/or scoring of SDMT are provided below

### (c) Exploratory Objective

The exploratory efficacy objective for this study is to evaluate the efficacy of a higher dose of ocrelizumab compared with the approved dose of ocrelizumab on the basis of, but not limited to, the following endpoints:
Change from baseline in EDSS score at each scheduled visit; Time to 20% increase in 12-week confirmed 9-HPT;
Time to ≥20% increase in 24-week confirmed 9-HPT; The following patient-reported outcomes:
   Change from baseline in MSIS-29 psychological scale at each scheduled visit;
   Change from baseline in Quality of Life in Neurological Disorders (Neuro-QoL) Upper Extremity Function Form at each scheduled visit
   Time to 8-point decrease in 12-Item Multiple Sclerosis Walking Scale (MSWS-12);
   Change from baseline in Modified Fatigue Impact Scale (MFIS) at each scheduled visit;
   Proportion of patients with no change, improvement or worsening in patient global impression of severity (PGI-S) at each scheduled visit;
   Proportion of patients with no change, improvement or worsening in patient global impression of change (PGI-C) at each scheduled visit;
   Proportion of patients with no change, improvement or worsening in patient global impression of change of the upper limb function (PGI-C-UL) at each scheduled visit;
Time to onset of cCDP12 and progression in cCDP individual components independent of protocol-defined relapses (PIRA);
Total number of new T1-hypo-intense lesions (black holes);
Volume of T1-hypo-intense lesions (black holes);
Volume of spinal cord (upper part of the spine);
Annualized protocol-defined relapse rate (ARR);
Time to onset of 12-week confirmed protocol-defined relapse associated worsening (RAW) and individual components;
Total number of new T2 lesions and enlarging T2 lesions per MRI scan over the 120-week treatment period and at each scheduled visit;
Total number of T1Gd⁺ lesions over the 120-week treatment period and at each scheduled visit.

Additional details regarding the assessment of the endpoints listed above are described below.

### (d) Subgroup Analyses

Subgroup analyses are performed based on the following parameters:
Randomization stratification factors;
EDSS
T1 Gd⁺ lesion count
T2 lesion count
Time since onset of MS symptoms

### (e) Safety Objectives

The safety objective for this study is to evaluate the safety profile of a higher dose of ocrelizumab compared with the approved dose of ocrelizumab as well as the overall safety profile and safety profile by treatment arm over time, on the basis of the following endpoints:
Incidence and severity of adverse events, with severity determined according to the National Cancer Institute's Common Terminology Criteria for Adverse Events (NCI CTCAE) v5.0 (see ctep(dot)cancer(dot)gov/protocolDevelopment/electronic_applications/ctc(dot)htm);
Change from baseline in clinical laboratory test results (including hematology, chemistry, and Ig levels);
Change from baseline in vital signs (including systolic and diastolic blood pressure, and pulse rate) following study treatment administration

### (f) Pharmacokinetic and Pharmacodynamic Objectives

The PK objective for this study is to assess the exposure to ocrelizumab in serum in all patients in both study arms:
Serum concentration of ocrelizumab at specified time points, and derived PK parameters via the population PK approach

The exploratory PK objectives for this study are to evaluate a potential relationship between drug exposure and the efficacy and safety of ocrelizumab:
Correlation of ocrelizumab serum concentration with efficacy endpoints;
Correlation of ocrelizumab serum concentration with safety endpoints

The Pharmacodynamics (PD) objective for this study is to characterize the ocrelizumab PD profile on the basis of the following endpoints:
B-cell levels in blood (including comparing the degree of B-cell depletion between the doses);
Proportion of patients achieving 5 or less B-cells per microliter of blood;
Proportion of patients achieving 5 or less B-cells per microliter of blood in patients with the high versus low affinity Fcγ Receptor 3A (FcγR3A) genotype per arm.

### (g) Immunogenicity Objective

The immunogenicity objective for this study is to evaluate the immune response to ocrelizumab on the basis of the following endpoint:
Prevalence of anti-drug antibodies (ADAs) at baseline and incidence of ADAs during the study.

### (h) Biomarker Objective

The biomarker objectives for this study are to identify biomarkers that are predictive of response to a higher dose of ocrelizumab (i.e., predictive biomarkers), are early surrogates of efficacy, are associated with progression to a more severe disease state (i.e., prognostic biomarkers), are associated with acquired resistance to ocrelizumab, are associated with susceptibility to developing adverse events or can lead to improved adverse event monitoring or investigation (i.e., safety biomarkers), can provide evidence of ocrelizumab activity (i.e., PD biomarkers), or can increase the knowledge and understanding of disease biology and drug safety. The following biomarker analyses are implemented:
Levels of soluble biomarkers including but not limited to neurofilament light chain (NfL) and/or IL-6 in blood (plasma and/or serum);
Levels of blood B-cells based on a highly sensitive assay that can accurately measure below 5 B-cells per microliter in blood;
Levels of B or T cell subsets in blood, including but not limited to CD19⁺ IgD, CD27, CD38, CD4, CD8, CD3, parameters to identify B or T naïve, memory and/or B plasmablast/plasma cell subsets
DNA genotype of patients to include but not be limited to FcγR3A and human leukocyte antigen (HLA) genotype.

MS biomarkers in cerebrospinal fluid (CSF) assessed in screening samples required of patients without documented evidence of prior oligoclonal band (OCB) positivity, include but not be limited to measurement of OCBs, IgG index, and light chain immunoglobulins.

### II. Study Design

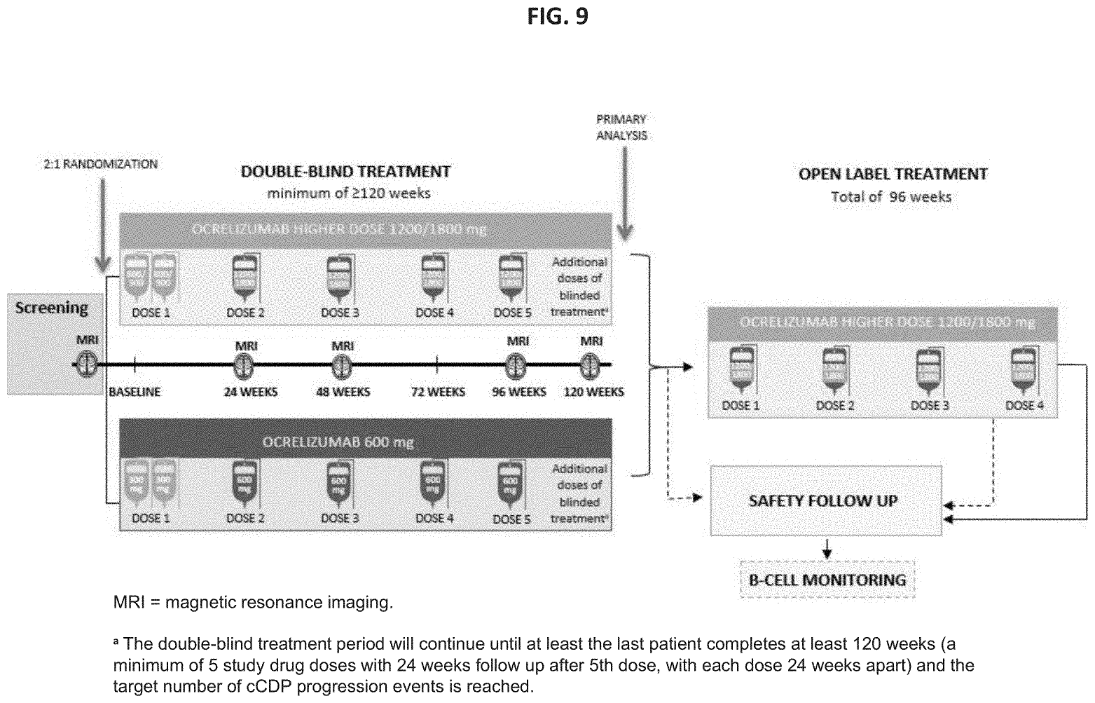

This study consists of the following phases: (i) a screening, (ii) double blind treatment (DBT) phase, (iii) an open-label extension (OLE) phase, and (iv) a safety follow-up (SFU) and B cell monitoring (BCM) phase. FIG. 9 presents an overview of the study design. Table F presents the overview of ocrelizumab dosing regimen during the double-blind treatment phase.

TABLE F
Overview of Ocrelizumab Dosing Regimen During the Double-Blind Treatment Phase

| | Double-Blind Treatment Phase | | | | | | |
|---|---|---|---|---|---|---|---|
| | Minimum 5 Treatment Doses (120 Weeks)^{a,b,c} | | | | | | |
| | | | | | | | N (additional |
| | | | | | | | treatment doses)^{a} |
| | Dose 1 | | Dose 2 | Dose 3 | Dose 4 | Dose 5 | (Every 24 wks) |
| | Day 1 | Day 15 | Wk 24 | Wk 48 | Wk 72 | Wk 96 | Wk 120+ |
| A OCR | 600 mg | 600 mg | 1200 mg | 1200 mg | 1200 mg | 1200 mg | 1200 mg |
| higher dose | | | | | | | |
| patients < | | | | | | | |
| 75 kg^{a} | | | | | | | |
| A OCR | 900 mg | 900 mg | 1800 mg | 1800 mg | 1800 mg | 1800 mg | 1800 mg |
| higher dose | | | | | | | |
| patients ≥ | | | | | | | |
| 75 kg^{d} | | | | | | | |
| B OCR | 300 mg | 300 mg | 600 mg | 600 mg | 600 mg | 600 mg | 600 mg |
| approved | | | | | | | |
| dose | | | | | | | |

OCR = ocrelizumab;
Wk = week.
Each study drug dose has a duration of 24 weeks (±5 days).
^{a}Enrolled patients undergo ocrelizumab (approved or higher dose) treatment of minimum of five treatment doses. When applicable, patients have subsequent treatment dosing that consists of the same dosing regimen, at 24-week intervals, until the end of the DBT period. The DBT period ends once the last patient completes at least 120 weeks (a minimum of five study drug doses with 24 weeks follow up after 5th dose, with each dose 24 weeks apart) and the target number of cCDP progression events is reached and primary analysis is performed.
^{b}After the first infusion of the first dose, an evaluation of retreatment criteria is performed before each subsequent infusion to ensure the patient remains eligible for further treatment.
^{c}A dose of 100 mg of methylprednisolone IV and oral or IV antihistamine (e.g., IV diphenhydramine 50 mg), or equivalent dose of alternative, is administered prior to ocrelizumab infusions. In patients where methylprednisolone is contraindicated, equivalent doses of other IV steroids (e.g., dexamethasone) are used as premedication.
^{d}The actual higher dose of ocrelizumab is assigned to patients as based on their body weight at baseline: 1200 mg (patient's body weight < 75 kg) or 1800 mg (patient's body weight ≥ 75 kg).

### (a) Overview of the Study Phases

### (i) Screening Phase

Patients providing informed consent undergo screening prior to the study drug administration. Eligible patients are randomized (2:1) in a blinded fashion to either the higher dose or the approved dose of ocrelizumab. Randomization is stratified by weight at baseline (<75 kg or ≥75 kg), region (U.S. or Rest of World [ROW]), EDSS (<4.0 vs. ≥4.0) and age (≤45 or >45). The sample size is approximately 786 patients (524 in the higher dose arm and 262 in the approved dose control arm). The subtype of RMS (i.e., RRMS or active SPMS, also known as “[a]SPMS”) is collected at screening for each patient and recorded.

The actual higher dose of ocrelizumab is assigned to patients as based on their body weight:
1200 mg ocrelizumab for patients with body weight <75 kg at baseline
1800 mg ocrelizumab for patients with body weight ≥75 kg at baseline

Throughout the study conduct, patients receive the dose assigned at baseline. Changes of the study drug dose assigned at baseline are not foreseen. Significant changes in patient's body weight during study are reported.

### (ii) Double-Blind Treatment Phase

Patients are treated for a minimum of 120 weeks (with a minimum of five study drug doses, 24-week follow up after fifth dose, and with each dose 24 weeks apart) or longer and the blinded treatment continues until at least 205 events of cCDP12 (i.e., 12-week composite confirmed disability progression, which is described in further detail below) occur in the study. The primary efficacy analysis is performed after the above-mentioned number of events has been reached. Each study dose period lasts for 24 weeks, starting from the study drug dose administration. Patients who prematurely discontinue from study treatment, including patients who start receiving alternative MS medication, remain in the main double-blind study phase and are followed for both efficacy and safety until the end of the double-blind phase (i.e., until the time of the primary analysis).

A minimum interval of 20 weeks typically occurs between the ocrelizumab second infusion of Dose 1 (i.e., infusion Day 15) and the next infusion of Dose 2 (Week 24). A minimum of 22 weeks typically occurs between ocrelizumab single infusions administered during Weeks 24, 48, 72, 96, and any dose thereafter. Treatment with ocrelizumab infusion typically occurs within 24 hours of randomization. If the ocrelizumab infusion at Week 24, 48, 72, 96, or any further infusion thereafter is not administered on the same study visit day, the infusion is given within the next 24 hours, provided that the patient still meets re-treatment criteria (see below). Whenever possible, infusion bags are prepared on the day of the infusion administration. Patients who cannot receive their infusion at the scheduled visit or within 24 hours of the visit are re-scheduled for a delayed dosing visit. Additional unscheduled visits for the assessment of potential relapses, new neurological symptoms, or safety events occur at any time.

(iii) Optional Open-Label (OLE) Extension Phase

If the result of the primary analysis is positive, eligible patients who have adhered to the DBT until the primary analysis and could benefit from a higher dose of ocrelizumab participate in an optional higher dose extension treatment (OLE phase). The OLE is carried out for approximately 96 weeks (4 doses in total) starting from the first OLE dose. The 96-week duration of the OLE phase serves to further evaluate long-term safety and efficacy of a higher dose of ocrelizumab. The currently approved 600 mg dose of ocrelizumab is not offered in this extension phase. During the OLE phase, patients originally randomized to the higher dose group continue with their assigned dose of ocrelizumab (either 1200 or 1800 mg). Patients who were assigned to the control group and received 600 mg ocrelizumab in the DBT are offered a higher dose of ocrelizumab, based on their body weight at OLE baseline. The blinding procedures are not necessary during the OLE phase. Efficacy assessments continue through the OLE phase.

### (iv) Safety Follow-Up (SFU) Phase and B-Cell Monitoring (BCM)

SFU phase begins after primary analysis results are available. Each patient is followed for safety for 48 weeks, starting from the last ocrelizumab dose received. Patients either enter the SFU phase if they prematurely discontinue randomized treatment in the DBT phase but do not reach the 48-week follow-up post-study drug discontinuation within DBT phase by the time DBT phase ends, or if they complete or prematurely discontinue the OLE phase.

Patients who discontinue ocrelizumab treatment during the DBT phase remain in the DBT phase until its conclusion and continue to be assessed for endpoints. This period of time within the DBT phase, where patients are not receiving an ocrelizumab infusion but are being assessed for the endpoints described above counts as part of the 48-week safety follow-up period. Patients who do not reach a 48-week period required for safety monitoring during the DBT phase transition to the SFU phase. Laboratory and safety assessments are performed during the clinic visits that occur every 12 weeks.

At the end of the required safety follow up (either within the DBT phase or the SFU phase), patients whose B-cell levels are not repleted to their baseline level or to the low level of normal (LLN), whichever is lower, move into the BCM phase. The study ends when all patients who are not treated with an alternative B-cell depleting therapy replete their B-cells to the baseline value or the lower limit of normal (whichever is lower).

### (b) Optional CSF Biomarker Substudy

The purpose of this optional substudy is to assess whether higher doses of ocrelizumab have a greater impact on B-cell depletion in the cerebrospinal fluid (CSF). The primary objectives of this substudy assess NfL (neurofilament light chain) levels and B-cell number in the CSF. Secondary and exploratory objectives assess the presence or absence of OCBs (oligoclonal bands), the exposure of ocrelizumab, specific subsets or types of B-cells present, and T-cells or other biomarkers in the CSF. Patients in this optional substudy undergo three lumbar punctures to obtain CSF at baseline pre-dose, Week 24, and Week 52. The CSF biomarker substudy enrolls up to 144 patients with RMS.

### (c) End of Study and Length of Study

The end of the DBT phase is defined as the date at which the last data point that is required for the primary efficacy analysis is received from the last patient. The end of the study occurs when all patients, who are not being treated with an alternative B-cell depleting therapy, replete their B-cells (i.e., B-cell level of the patient returns to the baseline value or the lower limit of normal, whichever is lower).

### III. Materials and Methods

### (a) Patients

This study enrolls patients with RMS. Approximately 786 patients are recruited into the study.

### (i) Inclusion Criteria

Patients meet the following criteria for study entry:
Signed informed written consent form (ICF);
Ages 18-55 years at time of signing ICF;
Ability to comply with the study protocol;
Diagnosis of RMS in accordance with the revised McDonald Criteria 2017 (Thompson et al. (2018) Lancet Neurol. 17:162-73);
At least two documented clinical attacks within the last 2 years prior to screening, or one clinical attack in the year prior to screening (with no relapse 30 days prior to screening and at baseline);
Patients must be neurologically stable for at least 30 days prior to randomization and baseline assessment;
Expanded Disability Status Scale (EDSS) score, at screening and baseline, from 0 to 5.5 inclusive;
Documented MRI of brain with abnormalities consistent with MS prior to screening;
Patients requiring symptomatic treatment for MS (e.g., fampridine, cannabis) and/or physiotherapy are treated at a stable dose during the screening period prior to the initiation of study drug on Day 1 and have a plan to remain at a stable dose for the duration of study treatment;
Patient do not initiate symptomatic treatment for MS or physiotherapy within 4 weeks of randomization.

For females of childbearing potential: agreement to remain abstinent (refrain from heterosexual intercourse) or use adequate contraceptive methods during the treatment period and for 6 or 12 months (as applicable by the ocrelizumab [Ocrevus] local label) after the final dose of ocrelizumab.
For female patients without reproductive potential: Females are enrolled if post-menopausal (i.e., spontaneous amenorrhea for the past year confirmed by a follicle-stimulating hormone [FSH] level; 40 mIU/mL) unless the patient is receiving a hormonal therapy for her menopause or if surgically sterile (i.e., hysterectomy, complete bilateral oophorectomy).

### (ii) Exclusion Criteria

Patients who meet any of the following criteria are excluded from study entry:
History of primary progressive MS at screening;
Any known or suspected active infection at screening or baseline (except nailbed infections), or any major episode of infection requiring hospitalization or treatment with IV anti microbials within 8 weeks prior to and during screening or treatment with oral anti microbials within 2 weeks prior to and during screening; \
History of confirmed or suspected progressive multifocal leukoencephalopathy (PML);
History of cancer, including hematologic malignancy and solid tumors, within 10 years of screening (basal or squamous cell carcinoma of the skin that has been excised and is considered cured and in situ carcinoma of the cervix treated with apparent success by curative therapy >1 year prior to screening is not exclusionary;
Immunocompromised state, defined as one or more of the following: CD4 count <250/μL or absolute neutrophil count <1.5×10³/μL or serum IgG <4.6 g/L;
Receipt of a live or live-attenuated vaccine within 6 weeks prior to randomization (influenza vaccination is permitted if the inactivated vaccine formulation is administered);
Inability to complete an MRI (contraindications for MRI, including but not restricted to, pacemaker, cochlear implants, intracranial vascular clips, surgery within 6 weeks of entry in the study, coronary stent implanted within 8 weeks prior to the time of the intended MRI, etc.) or contraindication to gadolinium administration;
Contraindications to mandatory pre-medications (i.e., corticosteroids and antihistamines) for infusion related reactions, including uncontrolled psychosis for corticosteroids or closed-angle glaucoma for antihistamines);
Known presence of other neurologic disorders, including, but not limited to, the following:
   History of ischemic cerebrovascular disorders (e.g., stroke, transient ischemic attack) or ischemia of the spinal cord;
   History or known presence of CNS or spinal cord tumor (e.g., meningioma, glioma);
   History or known presence of potential metabolic causes of myelopathy (e.g., untreated vitamin B12 deficiency);
   History or known presence of infectious causes of myelopathy (e.g., syphilis, Lyme disease, human T-lymphotropic virus type 1, herpes zoster myelopathy);
   History of genetically inherited progressive CNS degenerative disorder (e.g., hereditary paraparesis, mitochondrial myopathy, encephalopathy, lactic acidosis, stroke [MELAS] syndrome);
   Neuromyelitis optica spectrum disorders;
   History or known presence of systemic autoimmune disorders potentially causing progressive neurologic disease (e.g., lupus, anti-phospholipid antibody syndrome, Sjögren syndrome, Behçet disease);
   History or known presence of sarcoidosis; and
   History of severe, clinically significant brain or spinal cord trauma (e.g., cerebral contusion, spinal cord compression);
Any concomitant disease that requires chronic treatment with systemic corticosteroids or immunosuppressants during the course of the study;
Significant, uncontrolled disease, such as cardiovascular (including cardiac arrhythmia), pulmonary (including obstructive pulmonary disease), renal, hepatic, endocrine or gastrointestinal, or any other significant disease that precludes patient from participating in the study;
History of or currently active primary or secondary (non-drug-related) immunodeficiency;
Pregnant or breastfeeding or intending to become pregnant during the study or 6 or 12 months (as applicable from the local label for ocrelizumab) after final dose of the study drug (females of childbearing potential must have a negative serum and urine pregnancy test result prior to initiation of study drug (negative serum β-hCG measured at screening and negative urine β-hCG at baseline);
Lack of peripheral venous access;
History of alcohol or other drug abuse within 12 months prior to screening;
Treatment with any investigational agent within 24 weeks prior to screening (Visit 1) or five half-lives of the investigational drug (whichever is longer), or treatment with any experimental procedure for MS (e.g., treatment for chronic cerebrospinal venous insufficiency);
Previous use of anti-CD20s is allowed if the last infusion was more than 2 years before screening, B-cell count is normal, and the stop of the treatment was not motivated by safety reasons or lack of efficacy;
Previous use of mitoxantrone, cladribine, atacicept, and alemtuzumab;
Previous treatment with any other immunomodulatory or immunosuppressive medication not already listed above without appropriate washout as described in the applicable local label.
If the washout requirements are not described in the applicable local label, then the washout period must be five times the half-life of the medication. The PD effects of the previous medication must also be considered when determining the required time for washout (patients screened for this study are not withdrawn from therapies for the sole purpose of meeting eligibility for the trial);
Any previous treatment with bone marrow transplantation and hematopoietic stem cell transplantation;
Any previous history of transplantation or anti-rejection therapy;
Treatment with IV Ig or plasmapheresis within 12 weeks prior to randomization;
Systemic corticosteroid therapy within 4 weeks prior to screening (for a patient to be eligible, systemic corticosteroids are not to be administered between screening and baseline);
Positive screening tests for active, latent, or inadequately treated hepatitis B, as evidenced by either of the following: (a) positive hepatitis B surface antigen or (b) positive hepatitis B core antibody (total HBcAb) and detectable hepatitis B virus DNA;
Sensitivity or intolerance to any ingredient (including excipients) of ocrelizumab;
Any additional exclusionary criterion as per ocrelizumab (Ocrevus) local label, if more stringent than the above.

### (ii) Eligibility Criteria for Open-Label Extension (OLE) Phase

Patients meet the following criteria in order to participate in the OLE phase:
Complete the DBT phase of the trial and potentially benefit from treatment with a higher dose of ocrelizumab (patients who withdraw from study treatment, including patients who receive another disease-modifying therapy are not allowed to enter the OLE phase);
Able and willing to provide written informed consent to participate in the OLE phase and to comply with the study protocol;
Meet the re-treatment criteria for ocrelizumab (see below);
For women of childbearing potential: agreement to remain abstinent (refrain from heterosexual intercourse) or use adequate contraceptive methods during the treatment period and for 6 or 12 months (as applicable by the ocrelizumab [Ocrevus] local label) after the final dose of ocrelizumab;
Female patients without reproductive potential are enrolled if post-menopausal (i.e., spontaneous amenorrhea for the past year confirmed by a FSH level >40 mIU/mL) unless the patient is receiving a hormonal therapy for her menopause or if surgically sterile (i.e., hysterectomy, complete bilateral oophorectomy).

### (b) Method of Treatment Assignment and Blinding

### (i) Treatment Assignment

This is a randomized, double-blind study. After initial written informed consent has been obtained, all screening procedures and assessments have been completed, and eligibility has been established for a patient, the patient is randomly assigned to one of two treatment arms: (a) higher dose of ocrelizumab or (b) approved dose of ocrelizumab. Randomization of approximately 780 patients occurs in a 2:1 ratio (higher dose to approved dose, respectively) through use of a permuted-block randomization method to ensure a balanced assignment to each treatment arm. Randomization is stratified according to the following criteria:
Weight (<75 kg vs. ≥75 kg)
Region (United States vs. ROW)
EDSS (<4 vs. ≥4)
Age (≤45 years vs. >45 years)

### (ii) Blinding

Study site personnel and patients are blinded to treatment assignment during the study. The Sponsor and its agents are also blinded to treatment assignment, with the exception of individuals who require access to patient treatment assignments to fulfill their job roles during a clinical trial. Any unblinding at the investigating site is documented in the study report with the date, reason for identifying the assigned treatment/drug dose and name of the persons who request the unblinding. To prevent potential unblinding as a result of adverse events or laboratory changes, a “dual assessor” approach is used to evaluate efficacy and safety. Each site has two blinded investigators: a principal or Treating Investigator, who makes treatment decisions, and a rating or Examining Investigator, who assesses efficacy. The Efficacy Investigator is not involved in the medical management of the patient.

### (c) Study Treatment and Other Treatments Relevant to the Study Design

The Investigational Medicinal Product (IMP) for this study is ocrelizumab and matching placebo used to maintain the blind. Premedications, such as methylprednisolone (or equivalent) and antihistamines (such as diphenhydramine or equivalent) are considered non-investigational medicinal products (NIMPs).

### (i) Ocrelizumab and Placebo Vials

Ocrelizumab is supplied in 15 cc Type I glass vials as a sterile, single-use solution for IV infusion and contains no preservatives. Each vial contains 300 mg of ocrelizumab, at a nominal fill volume of 10 mL. The drug product is formulated as 30 mg/mL ocrelizumab in 20 mM sodium acetate at pH 5.3, with 106 mM trehalose dihydrate and 0.02% polysorbate 20. Ocrelizumab can contain fine translucent and/or reflective particles associated with enhanced opalescence. The solution is not used if discolored or if the solution contains discrete foreign particulate matter. Ocrelizumab solutions for IV administration are prepared by dilution of ocrelizumab in infusion bags containing 0.9% sodium chloride. The infusion solution must be administered using an infusion set with an in-line, sterile, non-pyrogenic, low-protein-binding filter (pore size of 0.2 micrometer or less). Ocrelizumab matching placebo vials are used in the study to enable blinding of the study drug doses across the study arms. These placebo vials have the same composition and configuration as the drug product but do not contain ocrelizumab. Each study medication kit contains 1 single-use vial of either 300 mg ocrelizumab or ocrelizumab placebo.

For the DBT, in Dose 1 consisting of two infusions 14 days apart, the following blinded study medication kits are dispensed according to the assigned treatment arm:
Infusion 300 mg: one ocrelizumab verum vial and two ocrelizumab placebo vials;
Infusion 600 mg: two ocrelizumab verum vials and one ocrelizumab placebo vial;
Infusion 900 mg: three ocrelizumab verum vials.

For each of the subsequent study doses, the following blinded study medication kits are dispensed for the first and second infusion bag according to the assigned treatment arm:
Infusion 600 mg: two ocrelizumab verum vials for the first infusion bag, and four ocrelizumab placebo vials for the second infusion bag;
Infusion 1200 mg: two ocrelizumab verum vials for the first infusion bag, and two ocrelizumab verum vials plus two ocrelizumab placebo vials for the second infusion bag;
Infusion 1800 mg: two ocrelizumab verum vials for the first infusion bag, and four ocrelizumab verum vials for the second infusion bag.

In the OLE phase, ocrelizumab vials are not blinded.

### (ii) Non-Investigational Medicinal Products (NIMPs)

In this study, NIMPs include premedication to the ocrelizumab infusion. The following premedication are used:
Mandatory methylprednisolone (or equivalent);
Mandatory antihistaminic drug (e.g., diphenhydramine or equivalent);
Recommended oral analgesic/antipyretic (e.g., acetaminophen 1 g).

To reduce potential infusion related reactions, all patients receive mandatory prophylactic treatment with 100 mg of methylprednisolone administered by slow IV infusion, to be completed approximately 30 minutes before the start of each ocrelizumab infusion. In the rare case when the use of methylprednisolone is contraindicated for the patient, an equivalent dose of an alternative steroid is used. Additionally, a mandatory oral or IV antihistaminic drug (i.e., diphenhydramine 50 mg or an equivalent dose of an alternative) must be administered approximately 30-60 minutes prior to the start of each ocrelizumab infusion. An analgesic/antipyretic (i.e., acetaminophen/paracetamol 1 g) is also considered. Hypotension (a symptom of IRR) can occur during study drug IV infusions. Therefore, withholding antihypertensive treatments is considered for 12 hours prior to and throughout each study drug infusion.

### (d) Retreatment Criteria for Ocrelizumab

Prior to re-treatment (i.e., re-administration of ocrelizumab to study participants at Week 24, 48, 72, 96, etc., the following conditions are met:
Absence of severe allergic or anaphylactic reaction to a previous ocrelizumab infusion;
Absence of any significant or uncontrolled medical condition or treatment-emergent, clinically significant laboratory abnormality;
Absence of active infection;
ANC≥1.5×10³/μL;
CD4 cell count ≥250/μL;
IgG ≥3.3 g/L

If any of these are not met prior to re-dosing, further administration of ocrelizumab is resolved or held indefinitely.

### IV. Study Assessments

### (a) Physical Examination and Vital Signs

The medical history of each patient (including, but not limited to clinically significant diseases, surgeries, cancer history, etc.) is recorded at screening and baseline. All medications (e.g., prescription drugs, over-the-counter medications, herbal/homeopathic remedies, nutritional supplements, etc.) used by the patient within 7 days prior to initiation of study treatment and ongoing therapies (e.g., physiotherapy) are recorded. Vaccinations received within 10 years prior to screening and throughout the study are recorded. At the time of each follow-up physical examination at specified time points throughout the study, an interval medical history is obtained and changes in medications are recorded.

Any previous medications taken for the treatment of MS since disease onset, including their start and end dates, and medications taken for the symptoms of MS in the 3-month period prior to the baseline visit is recorded at the baseline visit.

Vital signs, which are taken on infusion days prior to infusion, include measurements of systolic and diastolic blood pressure when the patient is in a seated position, pulse rate, and temperature. Additional vital signs readings are taken post-infusion and at the discretion of the investigator.

### (b) Neurological Examination

A neurological examination is performed at every planned visit. During an unscheduled visit, the neurological examination is performed only if deemed necessary. A neurologic examination includes assessment of mental status, level of consciousness, cranial nerve function, motor function, sensory function, reflexes, and coordination. Any abnormality identified at baseline is recorded. A neurological evaluation is scheduled promptly at performed within 7 days of newly identified or worsening neurological symptoms.

### (c) Assessment of Disability

Disability in MS is commonly measured by the Expanded Disability Status Scale (EDSS). EDSS is administered at the time points throughout the study. Additional EDSS assessments for individual patients are be requested between visits (e.g., during an MS relapse, neurological worsening, etc.).

### (d) Assessment of Relapse

Patients are evaluated for relapse at each visit throughout the study and, if necessary, at unscheduled visits to confirm relapse occurring between the visits.

A relapse is defined as the occurrence of new or worsening neurological symptoms attributable to MS and immediately preceded by a relatively stable or improving neurological state of least 30 days. Symptoms must persist for >24 hours and are not attributable to confounding clinical factors (e.g., fever, infection, injury, adverse reactions to concomitant medications). The new or worsening neurological symptoms are accompanied by objective neurological worsening consistent with an increase of at least one of the following:
Half a step (0.5 point) on the EDSS;
Two points on one of the selected FSS as listed below;
One point on two or more of the selected FSS as listed below:

The change must affect the following selected FSS: pyramidal, ambulation, cerebellar, brainstem, sensory, or visual. Episodic spasms, sexual dysfunction, fatigue, mood change, or bladder or bowel urgency or incontinence does not suffice to establish a relapse. Clinical relapses are recorded.

### (e) MRI Sequences

MRI is used to monitor central nervous system (CNS) lesions in patients. MRI scans of the brain, and also of the upper part of the spine if technically possible, are obtained in all patients at study visits. During the screening phase, one MRI scan is performed, and it serves as a baseline scan. quality and for potential re-scans if needed. Post-baseline, MRI scans are obtained in all patients at specified time points throughout the study. MRI assessments include, are not limited to, T1-weighted scans before and after injection of Gd contrast, fluid-attenuated inversion recovery, proton density-weighted, and T2-weighted scans.

### (f) Clinical Outcome Assessments

Patient reported outcome (PRO) measures (i.e., MSIS-29 v2, PGI-S, PGI-C, Neuro-QoL-Upper-Extremity, PGIC-UL, MSWS-12; MFIS, and EQ-5D-5L), clinician reported outcome (ClinRO) measures, and performance outcome (PerfO) measures ae completed to assess the treatment benefit of a higher dose of ocrelizumab relative to the approved dose. All measures are completed in their entirety at specified time points throughout the study.

### (i) Clinician Reported Outcome Assessments and Performance Outcomes

### (A) Expanded Disability Status Scale (EDSS)

The EDSS is the most commonly used ClinRO measure for quantifying changes in the disability level of patients with MS over time. The EDSS is a disability scale that ranges in 0.5-point steps from 0 (normal) to 10.0 (death) (see Kurtzke J F. Rating neurologic impairment in multiple sclerosis: an expanded disability status scale (EDSS). Neurol 1983; 33:1444-52; and Kappos L. Standardized neurological examination and assessment of Kurtzke's functional systems and expanded disability status scale. Slightly modified from J. F. Kurtzke, Neurology 1983:33, 1444-52. Neurology, University Hospital Basel, Switzerland: Neurostatus Scoring Definitions, 2011). The EDSS is based on a standard neurological examination, incorporating functional systems (visual, brainstem, pyramidal, cerebellar, sensory, bowel and bladder, and cerebral [or mental]) that are rated and then scored as a FSS (functional system score), and ambulation, which is scored as ambulation score. Each FSS is an ordinal clinical rating scale ranging from 0 to 5 or 6 and an ambulation score that is rated from 0 to 16. These ratings are then used in conjunction with observations, as well as information, concerning ambulation and use of assistive devices to determine the total EDSS score. Neurostatus-eEDSS definitions and calculating algorithms are used in this study (D'Souza M, Yaldizli Ö, John R, et al. Neurostatus e-Scoring improves consistency of Expanded Disability Status Scale assessments: A proof of concept study. Mult Scler Houndmills Basingstoke Engl. 2017; (4):597-603).

### (B) 9-Hole Peg Test (9-HPT)

The 9-HPT is a performance measure used to assess upper extremity (arm and hand) function (Goodkin D E, Hertsgaard D, Seminary J. Upper extremity function in multiple sclerosis: improving assessment sensitivity with Box-and-Block and Nine-Hole Peg Tests. Arch Phys Med Rehabil 1988; 69:850-54; Fischer J S, Rudick R A, Cutter G R, et al. The Multiple Sclerosis Functional Composite Measure (MSFC): an integrated approach to MS clinical outcome assessment. Mult Scler 1999; 5:244-50). The test consists of a container containing nine pegs and a wood or plastic block containing nine empty holes. The patient is to pick up each of the nine pegs one at a time and as quickly as possible place them in the nine holes. Once all the pegs are in the holes, the patient is to remove them again one at a time as quickly as possible and replace them into the container. The total time to complete the task is recorded. Both the dominant and non-dominant hands are tested twice (two consecutive trials of the dominant hand, followed immediately by two consecutive trials of the non-dominant hand). A 20% change from baseline is typically considered clinically meaningful (Feys P, Lamers I, Francis G, et al. The Nine-Hole Peg Test as a manual dexterity performance measure for multiple sclerosis. Multiple Sclerosis Journal 2017; 23(5):711-20).

### (C) Timed 25-Foot Walk Test (T25FWT)

The T25FWT test is a performance measure used to assess walking speed based on a timed 25-foot walk. The patient is directed to start at one end of a clearly marked 25-foot course and is instructed to walk 25 feet as quickly and safely as possible. The examining investigator times the patient from the start of the walk to the end of the 25 feet. The task is immediately administered again by having the patient walk back the same distance. The score for the T25FWT is the average of the two completed trials. Use of assistive devices (i.e., cane or wheelchair) is permitted when performing the task. The same assistive device is used at each study visit. Circumstances that affect the patient's performance are recorded. It is also recorded if the patient cannot complete the T25FWT twice. The T25FWT is administered as described in the MSFC Administration and Scoring Manual (see www(dot)nationalmssociety(dot)org/nationalmssociety/media/msnationalfiles/brochures/10-2-3-31-msfc_manual_and_forms(dot)pdf). A 20% change from baseline of the averaged T25FWT is typically considered clinically meaningful (www(dot)ema(dot)europa(dot)eu/en/documents/scientificguideline/draft-qualification-opinion-multiple-sclerosis-clinical-outcomeassessment-mscoa_en(dot)pdf and Hobart J, Blight A R, Goodman, A, et al. Timed 25-foot walk: direct evidence that improving 20% or greater is clinically meaningful in MS. Neurology 2013; 80(16):1509-17). The T25FWT is administered at specified time points throughout the study.

### (D) Symbol Digit Modalities Test (SDMT)

The SDMT is a performance measure that has demonstrated sensitivity in detecting not only the presence of cognitive impairment but also changes in cognitive functioning over time and in response to treatment (Smith A. Symbol digit modalities test: manual. Los Angeles: Western Psychological Services, 1982). The SDMT is recognized as being particularly sensitive to slowed processing of information that is commonly seen in MS (Benedict R H, DeLuca J, Phillips G, et al. Validity of the Symbol Digit Modalities Test as a cognition performance outcome measure for multiple sclerosis. Mult Scler 2017; 23(5):721-33). Briefly, using a reference key, the patient has 90 seconds to pair specific numbers with given geometric figures. Responses are collected orally. A four-point change from baseline is typically considered clinically meaningful (Benedict R H, DeLuca J, Phillips G, et al. Validity of the Symbol Digit Modalities Test as a cognition performance outcome measure for multiple sclerosis. Mult Scler 2017; 23(5):721-33). SDMT is administered at specified time points throughout the study.

### Example 4: A Phase IIIb Multicenter, Randomized, Double-Blind, Controlled Study to Evaluate the Efficacy, Safety and Pharmacokinetics of a Higher Dose of Ocrelizumab in Adults with Primary Progressive Multiple Sclerosis (PPMS)

This example describes a Phase IIIb, randomized, double blind, controlled, parallel group, multicenter study to evaluate efficacy, safety and pharmacokinetics of a higher dose of ocrelizumab (1200 mg [patient's body weight <75 kg] or 1800 mg [patient's body weight ≥75 kg]) per IV infusion every 24 weeks (6 months) in patients with PPMS, in comparison to the approved 600 mg dose of ocrelizumab.

### I. Efficacy Objectives

### (a) Primary Efficacy Objective

The primary efficacy objective is to demonstrate the superiority of a higher dose of ocrelizumab over the approved dose of ocrelizumab as assessed by risk reduction in composite confirmed disability progression (cCDP) sustained for at least 12 weeks.

The comparison of interest is the difference in time to 12-week cCDP (cCDP12), as expressed by the hazard ratio. The primary comparison is made regardless of adherence to the randomized treatment or use of alternative MS treatment.

Time to onset of cCDP is defined as the first occurrence of a confirmed progression event according to at least one of the following three criteria:
CDP, defined as a sustained increase from baseline in EDSS score of ≥1.0 point in patients with a baseline EDSS score of ≤5.5 or a sustained increase ≥0.5 points in patients with a baseline EDSS score of >5.5, or
A sustained increase of ≥20% from baseline in T25FWT score, or
A sustained increase of ≥20% from baseline in time to complete the 9-HPT score.

### (b) Secondary Efficacy Objective

The secondary efficacy objective is to demonstrate superiority of a higher dose of ocrelizumab over the approved dose of ocrelizumab on the basis of the following endpoints:
Time to onset of 24-week cCDP (cCDP24);
Time to onset of 12-week CDP (CDP12);
Time to onset of 24-week CDP (CDP24);
Time to ≥20% increase in 12-week confirmed T25FWT;
Time to ≥20% increase in 24-week confirmed T25FWT;
Time to ≥20% increase in 12-week confirmed 9-HPT;
Time to ≥20% increase in 24-week confirmed 9-HPT;
Change from baseline in the Multiple Sclerosis Impact Scale (MSIS-29) physical scale at Week 120;
Percent change in total brain volume from Week 24 to Week 120;
Time to 12-week confirmed 4-point worsening in Symbol Digit Modality test (SDMT).

For all time to event endpoints, the comparison of interest is the difference in time to event between treatment arms, as expressed by the hazard ratio. For all other endpoints, the comparison of interest is the difference in variable means between treatment arms. All comparisons, except for the MRI endpoint (i.e., the change in brain volume), are made regardless of adherence to the randomized treatment or use of alternative MS treatment. For the MRI endpoint, the comparison is made as if no treatment discontinuation or switch to alternative MS treatment occurs.

### (c) Exploratory Efficacy Objective

The exploratory efficacy objective for this study is to evaluate the efficacy of a higher dose of ocrelizumab compared with the approved dose of ocrelizumab on the basis of, but not limited to, the following endpoints:
Change from baseline in EDSS score at each scheduled visit;
The following patient-reported outcomes:
   Change from baseline in MSIS-29 psychological scale at each scheduled visit;
   Change from baseline in Quality of Life in Neurological Disorders (Neuro-QoL) Upper Extremity Function Form at each scheduled visit;
   Time to 8-point decrease in 12-Item Multiple Sclerosis Walking Scale (MSWS-12);
   Change from baseline in Modified Fatigue Impact Scale (MFIS) at each scheduled visit;
   Proportion of patients with no change, improvement or worsening in patient global impression of severity (PGI-S) at each scheduled visit;
   Proportion of patients with no change, improvement or worsening in patient global impression of change (PGI-C) at each scheduled visit;
   Proportion of patients with no change, improvement or worsening in patient global impression of change (PGI-C-UL) at each scheduled visit;
Total number of new T1-hypo-intense lesions (black holes);
Volume of T1-hypo-intense lesions (black holes);
Volume of spinal cord (the upper part of the spine);
Total number of new or enlarging T2 lesions per MRI scan over the 120-week treatment period and at each scheduled visit;
Total number of T1Gd⁺ lesions over the 120-week treatment period and at each scheduled visit

### (d) Safety Objectives

The safety objective for this study is to evaluate the safety profile of a higher dose of ocrelizumab compared with the approved dose of ocrelizumab as well as the overall safety profile and safety profile by treatment arm over time, on the basis of the following endpoints:
Incidence and severity of adverse events, with severity determined according to the National Cancer Institute's Common Terminology Criteria for Adverse Events (NCI CTCAE) v5.0 (see ctep(dot)cancer(dot)gov/protocolDevelopment/electronic_applicationsictc(dot)htm);
Change from baseline in clinical laboratory test results (including hematology, chemistry, and Ig levels);
Change from baseline in vital signs (including systolic and diastolic blood pressure, and pulse rate) following study treatment administration

### (e) Pharmacokinetic and Pharmacodynamic Objectives

The PK objective for this study is to assess the exposure to ocrelizumab in serum in all patients in both study arms:
Serum concentration of ocrelizumab at specified time points, and derived PK parameters via the population PK approach

The exploratory PK objectives for this study are to evaluate a potential relationship between drug exposure and the efficacy and safety of ocrelizumab:
Correlation of ocrelizumab serum concentration with efficacy endpoints Correlation of ocrelizumab serum concentration with safety endpoints
The pharmacodynamic (PD) objective for this study is to characterize the ocrelizumab PD profile on the basis of the following endpoints:
B-cell levels in blood (including comparing the degree of B-cell depletion between the doses);
Proportion of patients achieving 5 or less B-cells per microliter of blood
Proportion of patients achieving 5 or less B-cells per microliter of blood in patients with the high versus low affinity Fcγ Receptor 3A (FcγR3A) genotype per arm

### (f) Immunogenicity Objective

The immunogenicity objective for this study is to evaluate the immune response to ocrelizumab on the basis of the following endpoint:
Prevalence of anti-drug antibodies (ADAs) at baseline and incidence of ADAs during the study

### (g) Biomarker Objective

The biomarker objectives for this study are to identify biomarkers that are predictive of response to a higher dose of ocrelizumab (i.e., predictive biomarkers), are early surrogates of efficacy, are associated with progression to a more severe disease state (i.e., prognostic biomarkers), are associated with acquired resistance to ocrelizumab, are associated with susceptibility to developing adverse events or can lead to improved adverse event monitoring or investigation (i.e., safety biomarkers), can provide evidence of ocrelizumab activity (i.e., PD biomarkers), or can increase the knowledge and understanding of disease biology and drug safety. The following biomarker analyses are implemented:
Levels of soluble biomarkers including but not limited to neurofilament light chain (NfL) and/or IL-6 in blood (plasma and/or serum);
Levels of blood B-cells based on a highly sensitive assay that can accurately measure below 5 B-cells per microliter in blood;
Levels of B or T cell subsets in blood, including but not limited to CD19⁺ IgD, CD27, CD38, CD4, CD8, CD3, parameters to identify B or T naïve, memory and/or B plasmablast/plasma cell subsets.

DNA genotype of patients to include but not be limited to FcγR3A and human leukocyte antigen (HLA) genotype.

MS biomarkers in cerebrospinal fluid (CSF) assessed in screening samples required of patients without documented evidence of prior oligoclonal band (OCB) positivity, include but not be limited to measurement of OCBs, IgG index, and light chain immunoglobulins.

### II. Study Design

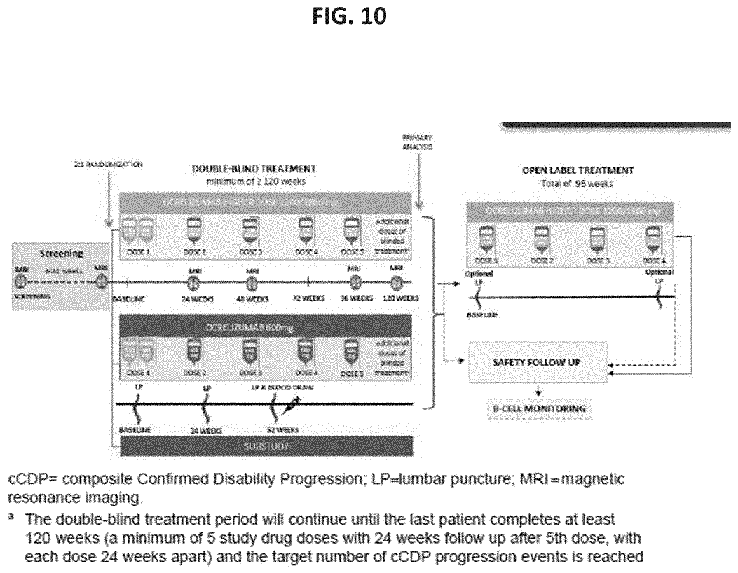

This study consists of the following phases: (i) a screening, (ii) double blind treatment (DBT) phase, (iii) an open-label extension (OLE) phase, (iv) a safety follow-up (SFU), and (v) a B cell monitoring phase. FIG. 10 presents an overview of the study design. Table F in Example 3 presents the overview of ocrelizumab dosing regimen during the double-blind treatment phase.

### (i) Screening

Approximately 687 eligible patients are randomized (2:1) in a blinded fashion to either the higher dose or the approved dose of ocrelizumab. The randomization is stratified by weight at baseline (<75 kg or ≥75 kg), region (U.S. or Rest of World [ROW]), sex (male vs. female), and age (≤45 or >45).

In order to assess patient's MRI activity level, two MRI scans (performed at least 6 weeks apart, but not more than 24 weeks apart) or one MRI with the sequences that can be compared with a historical MRI acquired in the previous 1 year (52 weeks) prior to screening are used to verify the MRI activity status of the patient. MRI activity is defined as the presence of any gadolinium-enhancing lesion(s) and/or new and/or enlarging T2 lesion(s) during the screening period. The MRI performed closer (i.e., from 6 weeks up to 10 days prior) to randomization is considered the baseline MRI for the study analyses. The sample size is approximately 699 patients (466 in the higher doses arm and 233 in the approved dose control arm).

The actual higher dose of ocrelizumab is assigned to patients as based on their body weight:
1200 mg ocrelizumab for patients with body weight <75 kg at baseline
1800 mg ocrelizumab for patients with body weight ≥75 kg at baseline

Throughout the study conduct, patients receive the dose assigned at baseline. Changes of the study drug dose assigned at baseline are not foreseen. Significant changes in patient's body weight during study are reported.

### (ii) Double-Blind Treatment Phase

Patients are treated for a minimum of 120 weeks (with a minimum of five study drug doses, 24-week follow up after fifth dose, and with each dose 24 weeks apart) or longer and the blinded treatment continues until at least 357 events of cCDP12 (i.e., 12-week composite confirmed disability progression, which is described in further detail in Example 3) occur in the study. The primary efficacy analysis is performed after the above-mentioned number of events has been reached. Each study dose period lasts for 24 weeks, starting from the study drug dose administration. Patients who prematurely discontinue from study treatment, including patients who start receiving alternative MS medication, remain in the main double-blind study phase and are followed for both efficacy and safety until the end of the double-blind phase (i.e., until the time of the primary analysis).

A minimum interval of 20 weeks occurs between the ocrelizumab second infusion of Dose 1 (i.e., infusion Day 15) and the next infusion of Dose 2 (Week 24). A minimum of 22 weeks occurs between ocrelizumab single infusions administered during Weeks 24, 48, 72, 96, and any dose thereafter. If the ocrelizumab infusion at Week 24, 48, 72, 96, or any further infusion thereafter is not administered on the same study visit day, the infusion is given within the next 24 hours, provided that the patient meets re-treatment criteria (see “Retreatment Criteria” in Example 3). Infusion bags are prepared on the day of the infusion administration. Patients who cannot receive their infusion at the scheduled visit or within 24 hours of the visit are re-scheduled for a delayed dosing visit. Additional unscheduled visits for the assessment of potential relapses, new neurological symptoms, or safety events occur at any time.

(iii) Optional Open-Label Extension Phase

If the result of the primary analysis is positive, eligible patients who have adhered to the DBT until the primary analysis and could benefit from a higher dose of ocrelizumab participate in an optional higher dose extension treatment (OLE phase). The OLE is carried out for approximately 96 weeks (4 doses in total) starting from the first OLE dose. The 96-week duration of the OLE phase serves to further evaluate long-term safety and efficacy of a higher dose of ocrelizumab. The currently approved 600 mg dose of ocrelizumab is not offered in this extension phase. During the OLE phase, patients originally randomized to the higher dose group continue with their assigned dose of ocrelizumab (either 1200 or 1800 mg). Patients assigned to the control group and received 600 mg ocrelizumab in the DBT are offered a higher dose of ocrelizumab, based on their body weight at OLE baseline. The blinding procedures are not necessary during the OLE phase. Efficacy assessments continue through the OLE phase.

### (iv) Safety Follow-Up (SFU) Phase and B-Cell Monitoring (BCM)

SFU phase begins after primary analysis results are available. Each patient is followed for safety for 48 weeks, starting from the last ocrelizumab dose received. Patients either enter the SFU phase if they prematurely discontinue randomized treatment in the DBT phase but do not reach the 48-week follow-up post-study drug discontinuation within DBT phase by the time DBT phase ends, or if they complete or prematurely discontinue the OLE phase. Patients who discontinue ocrelizumab treatment during the DBT phase remain in the DBT phase until its conclusion and continue to be assessed for endpoints. This period of time within the DBT phase, where patients are not receiving an ocrelizumab infusion are being assessed for the endpoints described above counts as part of the 48-week safety follow-up period. Patients who do not reach a 48-week period required for safety monitoring during the DBT phase transition to the SFU phase. Laboratory and safety assessments are performed during the clinic visits that occur every 12 weeks.

At the end of the required safety follow up (either within the DBT phase or the SFU phase), patients whose B-cell levels do not replete to their baseline level or the low level of normal (LLN), whichever is lower, move into the BCM phase. The study ends when all patients who were not treated with an alternative B-cell depleting therapy replete their B-cells to the baseline value or the lower limit of normal (whichever is lower).

### (b) Optional CSF Biomarker Substudy

The purpose of this optional substudy is to assess whether higher doses of ocrelizumab have a greater impact on B-cell depletion in the CSF. The primary objectives of this substudy assess NfL (neurofilament light chain) levels and B-cell number in the CSF. Secondary and exploratory objectives assess the presence or absence of OCBs (oligoclonal bands), the exposure of ocrelizumab, specific subsets or types of B-cells present, and T-cells or other biomarkers in the CSF. Patients in this optional substudy undergo three lumbar punctures to obtain CSF at baseline pre-dose, Week 24, and Week 52. The CSF biomarker substudy enrolls up to 144 patients with PPMS.

### (c) End of Study and Length of Study

The end of the DBT phase is defined as the date at which the last data point that is required for the primary efficacy analysis is received from the last patient. The end of the study occurs when all patients who are not being treated with an alternative B-cell depleting therapy, replete their B-cells (i.e., B-cell level of the patient returns to the baseline value or the lower limit of normal, whichever is lower).

### III. Materials and Methods

### (a) Patients

This study enrolls patients with PPMS. Approximately 699 patients are enrolled in this study.

### (i) Inclusion Criteria

Patients meet the following criteria for study entry:
Signed informed written consent form (ICF);
Ages 18-55 years at time of screening;
Ability to comply with the study protocol;
Diagnosis of PPMS, in accordance with the revised McDonald Criteria 2017 (Thompson A J, Banwell B L, Barkhof F, et al. Diagnosis of multiple sclerosis: 2017 revisions of the McDonald criteria. Lancet Neurol 2018; 17:162-73);
EDSS score at screening and baseline, from 3 to 6.5 inclusive
Score of ≥2.0 on the Functional Systems (FS) scale for the pyramidal system that was due to lower extremity findings
Patients requiring symptomatic treatment for MS (e.g., fampridine, cannabis) and/or physiotherapy are treated at a stable dose during the screening period prior to the initiation of study drug on Day 1 and have a plan to remain at a stable dose for the duration of study treatment;
Patients do not initiate symptomatic treatment for MS or physiotherapy within 4 weeks of randomization;
Patients must be neurologically stable for at least 30 days prior to randomization and baseline assessments;
Disease duration from the onset of MS symptoms:
   Less than 15 years in patients with an EDSS score at screening >5.0
   Less than 10 years in patients with an EDSS score at screening ≤5.0
Documented evidence of the presence of cerebrospinal fluid-specific oligoclonal bands (established by a historical lumbar puncture or presence at screening in a newly obtained CSF specimen (source documentation of historical laboratory results and method must be verified);
For females of childbearing potential: agreement to remain abstinent (refrain from heterosexual intercourse) or use adequate contraceptive methods during the treatment period and for 6 or 12 months (as applicable by the ocrelizumab [Ocrevus] local label) after the final dose of ocrelizumab;
For female patients without reproductive potential: Females are enrolled if post-menopausal (i.e., spontaneous amenorrhea for the past year confirmed by a follicle-stimulating hormone [FSH] level; 40 mIU/mL) unless the patient is receiving a hormonal therapy for her menopause or if surgically sterile (i.e., hysterectomy, complete bilateral oophorectomy).

### (b) Exclusion Criteria

Patients who meet any of the following criteria are excluded from study entry:
History of relapsing remitting or secondary progressive MS at screening;
Any known or suspected active infection at screening or baseline (except nailbed infections), or any major episode of infection requiring hospitalization or treatment with IV anti microbials within 8 weeks prior to and during screening or treatment with oral anti microbials within 2 weeks prior to and during screening;
History of confirmed or suspected progressive multifocal leukoencephalopathy (PML);
History of cancer, including hematologic malignancy and solid tumors, within 10 years of screening (basal or squamous cell carcinoma of the skin that has been excised and is considered cured and in situ carcinoma of the cervix treated with apparent success by curative therapy >1 year prior to screening is not exclusionary.
Immunocompromised state, defined as one or more of the following:
   CD4 count <250/μL or absolute neutrophil count <1.5×10³/μL or serum IgG <4.6 g/L
Receipt of a live or live-attenuated vaccine within 6 weeks prior to randomization (influenza vaccination is permitted if the inactivated vaccine formulation is administered);
Inability to complete an MRI (contraindications for MRI, including but not restricted to, pacemaker, cochlear implants, intracranial vascular clips, surgery within 6 weeks of entry in the study, coronary stent implanted within 8 weeks prior to the time of the intended MRI, etc.) or contraindication to gadolinium administration;
Contraindications to mandatory pre-medications (i.e., corticosteroids and antihistamines) for IRRs, including uncontrolled psychosis for corticosteroids or closed-angle glaucoma for antihistamines;
Known presence of other neurologic disorders, including, but not limited to, the following:
   History of ischemic cerebrovascular disorders (e.g., stroke, transient ischemic attack) or ischemia of the spinal cord;
   History or known presence of CNS or spinal cord tumor (e.g., meningioma, glioma);
   History or known presence of potential metabolic causes of myelopathy (e.g., untreated vitamin B12 deficiency);
   History or known presence of infectious causes of myelopathy (e.g., syphilis, Lyme disease, human T-lymphotropic virus type 1, herpes zoster myelopathy);
   History of genetically inherited progressive CNS degenerative disorder; (e.g., hereditary paraparesis, mitochondrial myopathy, encephalopathy, lactic acidosis, stroke [MELAS] syndrome)
   Neuromyelitis optica spectrum disorders;
   History or known presence of systemic autoimmune disorders potentially causing progressive neurologic disease (e.g., lupus, anti-phospholipid antibody syndrome, Sjögren syndrome, Behçet disease);
   History or known presence of sarcoidosis;
   History of severe, clinically significant brain or spinal cord trauma (e.g., cerebral contusion, spinal cord compression);
Any concomitant disease that requires chronic treatment with systemic corticosteroids or immunosuppressants during the course of the study;
Significant, uncontrolled disease, such as cardiovascular (including cardiac arrhythmia), pulmonary (including obstructive pulmonary disease), renal, hepatic, endocrine or gastrointestinal, or any other significant disease that preclude patient from participating in the study;
History of or currently active primary or secondary (non-drug-related) immunodeficiency;
Pregnant or breastfeeding or intending to become pregnant during the study or 6 or 12 months (as applicable from the local label for ocrelizumab) after final dose of the study drug
   Females of childbearing potential must have a negative serum and urine pregnancy test result prior to initiation of study drug (negative serum β-hCG measured at screening and negative urine β-hCG at baseline);
Lack of peripheral venous access;
History of alcohol or other drug abuse within 12 months prior to screening;
Treatment with any investigational agent within 24 weeks prior to screening (Visit 1) or five half-lives of the investigational drug (whichever is longer), or treatment with any experimental procedure for MS (e.g., treatment for chronic cerebrospinal venous insufficiency);
Previous use of anti-CD20s is allowed if the last infusion was more than 2 years before screening, B-cell count is normal, and the stop of the treatment was not motivated by safety reasons or lack of efficacy.
Previous use of mitoxantrone, cladribine, atacicept, and alemtuzumab;
Previous treatment with any other immunomodulatory or immunosuppressive medication not already listed above without appropriate washout as described in the applicable local label.
If the washout requirements are not described in the applicable local label, then the=washout period must be five times the half-life of the medication. The PD effects of the previous medication are considered when determining the required time for washout (patients screened for this study are withdrawn from therapies for the sole purpose of meeting eligibility for the trial);
Any previous treatment with bone marrow transplantation and hematopoietic stem cell transplantation;
Any previous history of transplantation or anti-rejection therapy;
Treatment with IV Ig or plasmapheresis within 12 weeks prior to randomization;
Systemic corticosteroid therapy within 4 weeks prior to screening (for a patient to be eligible, systemic corticosteroids are not to be administered between screening and baseline);
Positive screening tests for active, latent, or inadequately treated hepatitis B, as evidenced by either of the following: (a) positive hepatitis B surface antigen or (b) positive hepatitis B core antibody (total HBcAb) and detectable hepatitis B virus DNA;
Sensitivity or intolerance to any ingredient (including excipients) of ocrelizumab;
Any additional exclusionary criterion as per ocrelizumab (Ocrevus) local label, if more stringent than the above

### (i) Eligibility Criteria for Open-Label Extension (OLE) Phase

Patients meet the following criteria in order to participate in the OLE phase:
Complete the DBT phase of the trial and potentially benefit from treatment with a higher dose of ocrelizumab (patients who withdraw from study treatment, including patients who receive another disease-modifying therapy are not allowed to enter the OLE phase);
Able and willing to provide written informed consent to participate in the OLE phase and to comply with the study protocol;
Meet the re-treatment criteria for ocrelizumab (see “Retreatment Criteria” in Example 3);
For women of childbearing potential: agreement to remain abstinent (refrain from heterosexual intercourse) or use adequate contraceptive methods during the treatment period and for 6 or 12 months (as applicable by the ocrelizumab [Ocrevus] local label) after the final dose of ocrelizumab;
Female patients without reproductive potential are enrolled if post-menopausal (i.e., spontaneous amenorrhea for the past year confirmed by a FSH level >40 mIU/mL) unless the patient is receiving a hormonal therapy for her menopause or if surgically sterile (i.e., hysterectomy, complete bilateral oophorectomy).

### (c) Method of Treatment Assignment and Blinding

### (i) Treatment Assignment

This is a randomized, double-blind study. After initial written informed consent has been obtained, all screening procedures and assessments have been completed, and eligibility has been established for a patient, the patient is randomly assigned to one of two treatment arms: (a) higher dose of ocrelizumab or (b) approved dose of ocrelizumab. Randomization occurs in a 2:1 ratio (higher dose to approved dose, respectively) through use of a permuted-block randomization method to ensure a balanced assignment to each treatment arm. Randomization is stratified according to the following criteria:
Weight (<75 kg vs. ≥75 kg);
Region (United States vs. ROW);
Sex (male vs. female);
Age (≤45 years vs. >45 years)

### (ii) Blinding

Study site personnel and patients are blinded to treatment assignment during the study. The Sponsor and its agents are also blinded to treatment assignment, with the exception of individuals who require access to patient treatment assignments to fulfill their job roles during a clinical trial. Any unblinding at the investigating site is documented in the study report with the date, reason for identifying the assigned treatment/drug dose and name of the persons who request the unblinding. To prevent potential unblinding as a result of adverse events or laboratory changes, a “dual assessor” approach is used to evaluate efficacy and safety. Each site has two blinded investigators: a principal or Treating Investigator, who makes treatment decisions, and a rating or Examining Investigator, who assesses efficacy. The Efficacy Investigator is not involved in the medical management of the patient.

### (d) Study Treatment and Other Treatments Relevant to the Study Design

### (i) Ocrelizumab and Placebo Vials

Ocrelizumab is supplied, prepared for administration, and administered as described in the corresponding section in Example 3.

### (ii) Non-Investigational Medicinal Products (NIMPs)

In this study, NIMPs include premedication to the ocrelizumab infusion. The premedication used are described in the corresponding section in Example 3 and administered as described in the corresponding section in Example 3.

### (e) Retreatment Criteria for Ocrelizumab

Prior to re-treatment (i.e., re-administration of ocrelizumab to study participants at Week 24, 48, 72, 96, etc., patients meet the criteria described in the corresponding section in Example 3.

### IV. Study Assessments

### (a) Physical Examination and Vital Signs

The medical history, interval medical history, and vital signs of each patient is obtained and recorded as described in the corresponding section in Example 3.

### (b) Neurological Examination

Neurological examinations are performed and recorded as described in the corresponding section in Example 3.

### (c) Assessment of Disability

Disability in MS is commonly measured by the Expanded Disability Status Scale (EDSS). EDSS is administered at the time points throughout the study. Additional EDSS assessments for individual patients are be requested between visits (e.g., during an MS relapse, neurological worsening, etc.).

### (d) Assessment of Relapse

Although relapses are rare in patients with PPMS, patients are evaluated for relapse at each visit throughout the study and, if necessary, at unscheduled visits to confirm relapse occurring between the visits. A relapse is defined as described in the corresponding section in Example 3.

### (e) MRI Sequences

MRI is used to monitor central nervous system (CNS) lesions in patients. MRI scans of the brain, and also of the upper part of the spine if technically possible, are obtained in all patients at study visits.

Two MRI scans are performed prior to enrollment in order to assess the patient's MRI activity level. If a patient has had an MRI scan within 1 year from the start of the screening period and the scan is approved by the central reading facility, then only one MRI scan is performed during the screening period and it serves as a baseline scan. MRI activity is defined as the presence of any gadolinium-enhancing lesion(s) and/or new and/or enlarging T2 lesion(s) during the screening period. The MRI performed closer to randomization (i.e., either the second MRI scan at screening or the [only] screening MRI scan in the case where a historical scan is available) is considered as baseline MRI for the study analyses.

Post-baseline, MRI scans are obtained in all patients at specified time points throughout the study. MRI assessments include, are not limited to, T1-weighted scans before and after injection of Gd contrast, fluid-attenuated inversion recovery, proton density-weighted, and T2-weighted scans.

### (f) Clinical Outcome Assessments

Patient reported outcome (PRO) measures (i.e., MSIS-29 v2, PGI-S, PGI-C, Neuro-QoL-Upper-Extremity, PGIC-UL, MSWS-12; MFIS, and EQ-5D-5L), clinician reported outcome (ClinRO) measures, performance outcome (PerfO) measures, and MRIs are completed to assess the treatment benefit of a higher dose of ocrelizumab relative to the approved dose. All measures are completed in their entirety at specified time points throughout the study.

### (i) Clinician Reported Outcome Assessments and Performance Outcomes

### (A) Expanded Disability Status Scale (EDSS)

The EDSS is administered and scored as described in the corresponding section in Example 3.

### (B) 9-Hole Peg Test (9-HPT)

The 9-HPT is administered and scored as described in the corresponding section in Example 3.

### (C) Timed 25-Foot Walk Test (T25FWT)

The T25FWT is administered and scored as described in the corresponding section in Example 3.

### (D) Symbol Digit Modalities Test (SDMT)

The SDMT is administered and scored as described in the corresponding section in Example 3.

The examples, which are intended to be purely exemplary of the invention and should therefore not be considered to limit the invention in any way, also describe and detail aspects and embodiments of the invention discussed above. The foregoing examples and detailed description are offered by way of illustration and not by way of limitation.

1. A method of treating multiple sclerosis in a patient comprising
administering an effective amount of an anti-CD20 antibody to the patient to provide an initial anti-CD20 antibody dose of about 1.2 grams followed by a second anti-CD20 antibody dose of about 1.2 grams, the second dose not being provided until from about 24 weeks from the initial dose,
wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, and
wherein the patient weighs less than about 75 kg at the time of the first anti-CD20 antibody dose.

2. A method of treating multiple sclerosis in a patient comprising
administering an effective amount of an anti-CD20 antibody to the patient to provide an initial anti-CD20 antibody dose of about 1.2 grams followed by a second anti-CD20 antibody dose of about 1.2 grams, the second dose not being provided until from about 6 months from the initial dose,
wherein the anti-CD20 antibody comprises a V_{H} domain comprising the amino acid sequence set forth in SEQ ID NO: 8, a V_{L} domain comprising the amino acid sequence set forth in SEQ ID NO: 7, and a human IgG1 constant region, and
wherein the patient weighs less than about 75 kg at the time of the first anti-CD20 antibody dose.

3. The method of claim 1 or 2, wherein the initial anti-CD20 antibody dose comprises a first intravenous (IV) infusion and a second IV infusion of the anti-CD20 antibody, wherein the first IV infusion and second IV infusion of the anti-CD20 antibody are each about 0.6 grams.

4. The method of claim 1 or 2, wherein the initial anti-CD20 antibody dose comprises a single IV infusion of the anti-CD20 antibody, wherein the single IV infusion of the anti-CD20 antibody is about 1.2 grams.

5. The method of any one of claims 1-4, wherein the second anti-CD20 dose comprises a single IV infusion of the anti-CD20 antibody, wherein the single IV fusion of the anti-CD20 antibody is about 1.2 grams.

6. A method of treating multiple sclerosis in a patient comprising
administering an effective amount of an anti-CD20 antibody to the patient to provide an initial anti-CD20 antibody dose of about 1.8 grams followed by a second anti-CD20 antibody dose of about 1.8 grams, the second dose not being provided until from about 24 weeks from the initial dose,
wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and
wherein the patient weighs about 75 kg or more at the time of the first anti-CD20 antibody dose.

7. A method of treating multiple sclerosis in a patient comprising
administering an effective amount of an anti-CD20 antibody to the patient to provide an initial anti-CD20 antibody dose of about 1.8 grams followed by a second anti-CD20 antibody dose of about 1.8 grams, the second dose not being provided until from about 6 months from the initial dose,
wherein the anti-CD20 antibody comprises a V_{H} domain comprising the amino acid set forth in SEQ ID NO: 8, a V_{L} domain comprising the amino acid sequence set forth in SEQ ID NO: 7, and a human IgG1 constant region, and
wherein the patient weighs about 75 kg or more at the time of the first anti-CD20 antibody dose.

8. The method of claim 6 or 7, wherein the initial anti-CD20 antibody dose comprises a first intravenous (IV) infusion and a second IV infusion of the anti-CD20 antibody, wherein the first IV infusion and second IV infusion of the anti-CD20 antibody are each about 0.9 grams.

9. The method of claim 6 or 7, wherein the initial anti-CD20 antibody dose comprises a single IV infusion of the anti-CD20 antibody, wherein the single IV infusion of the anti-CD20 antibody is about 1.8 grams.

10. The method of any one of claims 6-9, wherein the second anti-CD20 antibody dose comprises a single IV infusion of the anti-CD20 antibody, wherein the single IV infusion of the anti-CD20 antibody is about 1.8 grams.

11. The method of claim 3 or 8, wherein the second IV infusion is administered from about 3 to 17 days from the time the first IV infusion is administered.

12. The method of claim 3 or 8, wherein the second IV infusion is administered from about 6 to 16 days from the time the first IV infusion is administered.

13. The method of claim 3 or 8, wherein the second IV infusion is administered from about 13 to 16 days from the time the first IV infusion is administered.

14. The method of claim 3 or 8, wherein the second IV infusion is administered 14 days from the time the first IV infusion is administered.

15. The method of claim 3 or 8, wherein the second IV infusion is administered two weeks from the time the first IV infusion is administered.

16. The method of any one of claims 1-15, further comprising providing a third anti-CD20 antibody dose.

17. The method of claim 16, wherein the third anti-CD20 antibody dose is provided about 24 weeks from the second dose.

18. The method of claim 16, wherein the third anti-CD20 antibody dose is provided about 6 months from the second dose.

19. The method of any one of claims 16-18, further comprising providing a fourth anti-CD20 antibody dose.

20. The method of claim 19, wherein the fourth anti-CD20 antibody dose is provided about 24 weeks from the third dose.

21. The method of claim 19, wherein the fourth anti-CD20 antibody dose is provided about 6 months from the third dose.

22. The method of any one of claims 19-21, further comprising providing a fifth anti-CD20 antibody dose.

23. The method of claim 22, wherein the fifth anti-CD20 antibody dose is provided about 24 weeks from the fourth dose.

24. The method of claim 22, wherein the fifth anti-CD20 antibody dose is provided about 6 months from the fourth dose.

25. The method of any one of claims 22-24, wherein subsequent anti-CD20 antibody doses following the fifth anti-CD20 antibody dose are administered at intervals of about 24 weeks.

26. The method of any one of claims 22-24, wherein subsequent anti-CD20 antibody doses following the fifth anti-CD20 antibody dose are administered at intervals of about 6 months.

27. The method of any one of claims 1-26, wherein the anti-CD20 antibody comprises a light chain comprising the amino acid sequence of SEQ ID NO: 9 and a heavy chain comprising the amino acid sequence of SEQ ID NO: 11.

28. The method of any one of claims 1-27, wherein the anti-CD20 antibody is ocrelizumab.

29. The method of any one of claims 1-28, wherein the multiple sclerosis is relapsing multiple sclerosis (RMS).

30. The method of claim 29, wherein the patient has RMS, and wherein treatment results in reduced risk of 12-week composite confirmed disability progression (cCDP 12).

31. The method of claim 29 or 30, wherein the patient has RMS, and wherein treatment results in one or more of:
(a) increase in time to onset of 24-week cCDP;
(b) increase in time to onset of 12-week confirmed disability progression (CDP);
(c) increase in time to onset of 24-week CDP;
(d) increase in time to >20% increase in 12-week confirmed timed 25 foot walk test (T25FWT);
(e) increase in time to >20% increase in 24-week confirmed T25FWT;
(f) decrease in the percent change in total brain volume after 24, 48, 72, 96, and 120 weeks of treatment; and
(g) increase in time to 12-week confirmed 4-point worsening in Symbol Digital Modality Test (SDMT).

32. The method of any one of claims 29-31, wherein the patient has RMS, and wherein treatment results in one or more of:
(A) reduction or no change in Expanded Disability Status Scare (EDSS) score;
(B) increase in time to >20% increase in 12-week confirmed 9-hole peg test (9-HPT);
(C) increase in time to >20% increase in 24-week confirmed 9-HPT;
(D) increase in time to onset of cCDP 12 and progression in cCDP individual components independent of relapses;
(E) reduction in new T1-hypointense lesions;
(F) reduction in volume of T1-hypointense lesions;
(G) reduction in spinal cord volume loss;
(H) reduction in annualized relapse rate (ARR);
(I) increase in time to onset of 12-week confirmed relapse-associated worsening (RAW) and individual components;
(J) reduction in number of new or enlarging T2 lesions over treatment period; and
(K) reduction in number of T1 Gd⁺ staining lesions over treatment period.

33. The method of any one of claims 1-28, wherein the multiple sclerosis is primary progressive multiple sclerosis (PPMS).

34. The method of claim 33, wherein the patient has PPMS, and wherein treatment results in reduced risk of 12-week composite confirmed disability progression (cCDP 12).

35. The method of claim 34, wherein the patient has PPMS, and wherein treatment results in one or more of:
(a) increase in time to onset of 24-week cCDP;
(b) increase in time to onset of 12-week confirmed disability progression (CDP);
(c) increase in time to onset of 24-week CDP;
(d) increase in time to >20% increase in 12-week confirmed timed 25 foot walk test (T25FWT);
(e) increase in time to >20% increase in 24-week confirmed T25FWT;
(f) increase in time to >20% increase in 12-week confirmed 9-hole peg test (9-HPT);
(g) increase in time to >20% increase in 24-week confirmed 9-HPT;
(h) decrease in loss of total brain volume during over treatment period following second ant-CD20 antibody dose; and
(i) increase in time to 12-week confirmed 4-point worsening in Symbol Digital Modality Test (SDMT).

36. The method of claim 34 or 35, wherein the patient has PPMS, and wherein treatment results in one or more of:
(A) a reduction or no change in Expanded Disability Status Scare (EDSS) score;
(B) reduction in new T1-hypointense lesions;
(C) reduction in volume of T1-hypointense lesions;
(D) reduction in spinal cord volume loss;
(E) reduction in number of new or enlarging T2 lesions over treatment period; and
(F) reduction in number of T1 Gd+ staining lesions over treatment period.

37. The method of any one of claims 1-36, wherein a second medicament is administered to the patient with the initial anti-CD20 antibody dose or later anti-CD20 antibody doses, wherein the anti-CD20 antibody is the first medicament.

38. The method of claim 37, wherein the second medicament is selected from the group consisting of an interferon, glatiramer acetate, a cytotoxic agent, a chemotherapeutic agent, mitoxantrone, methotrexate, cyclophosphamide, chlorambucil, azathioprine, gamma globulin, Campath, anti-CD4, cladribine, corticosteroid, mycophenolate mofetil (MMF), cyclosporine, a cholesterol-lowering drug of the statin class, estradiol, testosterone; a hormone replacement drug, a TNF inhibitor, a disease-modifying anti-rheumatic drug (DMARD), a non-steroidal anti-inflammatory drug (NSAID), levothyroxine, cyclosporin A, a somatastatin analogue, a cytokine or cytokine receptor antagonist, an anti-metabolite, an immunosuppressive agent, an integrin antagonist or antibody, an LF A −1 antibody, efalizumab, an alpha 4 integrin antibody, natalizumab, and another B-cell surface marker antibody.

39. The method of any one of claims 1-38, wherein the patient has never been previously treated with an anti-CD20 antibody.

40. The method of any one of claims 1-38, wherein the patient has received prior treatment with an anti-CD20 antibody

41. The method of any one of claims 1-36 and 39-40, wherein anti-CD20 antibody is the only medicament administered to the patient to treat multiple sclerosis.

42. An article of manufacture comprising:
(a) a container comprising an anti-CD20 antibody, which anti-CD20 antibody comprises a VH domain comprising the amino acid set forth in SEQ ID NO: 8, a VL domain comprising the amino acid sequence set forth in SEQ ID NO: 7, and a human IgG1 constant region; and
(b) a package insert with instructions for treating multiple sclerosis in a patient according to any one of the preceding claims.
